(19) United States
(12) Patent Application Publication
(10) Pub. No.: US 2006/0134102 A1
(43) Pub. Date: Jun. 22, 2006

(54) Lymphotoxin beta receptor agents in combination with chemotherapeutic agents

(75) Inventors: Doreen LePage, Pascoag, RI (US); Alan Gill, Reading, MA (US)

Correspondence Address: LAHIVE & COCKFIELD, 28 STATE STREET, BOSTON, MA, 02109, US

(73) Assignee: Biogen Idec MA Inc., Cambridge, MA (US)

(21) Appl. No.: 11/156,109

(22) Filed: Jun. 17, 2005

Related U.S. Application Data
(63) Continuation of application No. PCT/US03/41243, filed on Dec. 22, 2003.
(60) Provisional application No. 60/435,185, filed on Dec. 20, 2002.

(51) Int. Cl. A61K 39/395

(52) U.S. Cl. 424/133.1; 424/143.1

(57) ABSTRACT

This invention features combination therapies that include a composition that activates lymphotoxin-beta receptor signaling in combination with one or more other chemotherapeutic agents, as well as therapeutic methods and screening methods for identifying agents that in combination with a lymphotoxin-beta receptor agonist agent have a supra-additive effect on tumor inhibition.

### RELATED APPLICATIONS

This application is a continuation of International Patent Application Serial Number PCT/US03/041243, filed Dec. 22, 2003, which claims priority to U.S. Provisional Application No. 60/435185, filed Dec. 20, 2002. This application is also related to U.S. Provisional Application No. 60/435154, filed Dec. 20, 2002. The entire contents of each of these patents and patent applications are hereby incorporated herein by reference.

### FIELD OF THE INVENTION

This invention is in the fields of immunology and cancer diagnosis and therapy. More particularly it concerns the use of activating lymphotoxin beta receptor (LT-β-R) agents in combination with chemotherapeutic agent(s) in therapeutic methods.

### BACKGROUND OF THE INVENTION

Lymphotoxin beta receptor (referred to herein as LT-β-R) is a member of the tumor necrosis factor family which has a well-described role both in the development of the immune system and in the functional maintenance of a number of cells in the immune system including follicular dendritic cells and a number of stromal cell types (Crowe et al. (1994) Science 264:707; Browning et al. (1993) 72: 847; Browning et al. (1995) 154:33; Matsumoto et al. (1997) Immunol. Rev. 156:137). Activation of LT-β-R has been shown to induce the apoptotic death of certain cancer cell lines in vivo (PCT/US96/01386). Treatment with agonist LT-β-R activating agents, such as specific humanized anti-LT-β-R antibodies, would thus be useful for treating or reducing the advancement, severity or effects of neoplasia in subjects (e.g., humans). Cancer is one of the most prevalent health problems in the world today, affecting approximately one in five individuals in the United States. Thus, curbing the growth of neoplastic cells and treating various cancers is and will likely continue to be a major health need.

### SUMMARY OF THE INVENTION

The present invention provides, in part, methods of inhibiting tumor volume and treating cancer comprising the use of a lymphotoxin-beta receptor (LT-β-R) agonist and a chemotherapeutic agent, which is not a lymphotoxin receptor agonist. The combination of the agonist and agent achieves inhibition of a tumor greater than that expected by the simple addition of the effects of the agonist and agent alone. Such an effect is referred to herein as a “supra-additive” inhibition, and may be due to synergistic or potentiated interaction. The present invention also provides pharmaceutical compositions, delivery devices, and kits for use in the practice of the methods of the invention.

The invention provides a method for inhibiting tumor volume comprising administering an effective amount of a lymphotoxin-beta receptor (LT-β-R) agonist and an effective amount of at least one chemotherapeutic agent, wherein the administration of the LT-β-R agonist and the chemotherapeutic agent results in supra-additive inhibition of the tumor.

The invention also provides a method for inhibiting tumor volume comprising administering an effective amount of an anti-lymphotoxin-beta receptor (LT-β-R) antibody and an effective amount of at least one chemotherapeutic agent, wherein the administration of the anti-LT-β-R antibody and the chemotherapeutic agent results in supra-additive inhibition of the tumor.

The invention provides a pharmaceutical composition comprising an effective amount of a LT-β-R agonist, an effective amount of at least one chemotherapeutic agent, and a pharmaceutically acceptable carrier, which upon administration to a subject results in supra-additive inhibition of a tumor.

The invention also includes use of an effective amount of a lymphotoxin-beta receptor (LT-β-R) agonist and an effective amount of a chemotherapeutic agent, for the preparation of a medicament for the treatment of cancer, which upon administration to a subject results in supra-additive inhibition of a tumor.

In one embodiment of the invention, the supra-additive inhibition of the tumor is synergistic. In a further embodiment, the supra-additive inhibition of the tumor has a combination index of less than 1.00. In still another embodiment, the supra-additive inhibition of the tumor is potentiated. In a further embodiment of the invention, the supra-additive inhibition of the tumor has a P-value of less than 0.05.

In one embodiment of the invention, the LT-β-R agonist is an anti-LT-β-R antibody. In another embodiment, anti-LT-β-R antibody of the invention is a monoclonal antibody, wherein the monoclonal antibody is selected from the group consisting of: BKA11, CDH10, BCG6, AGH1, BDA8, CBE11 and BHA10. In stil another embodiment of the invention, the anti-LT-β-R antibody is a humanized antibody, including, for exmaple, huCBE11 and huBHA10. In still another embodiment, the anti-LT-β-R antibody of the invention is a multivalent anti-LT-β-R antibody. In one embodiment, the multivalent anti-LT-β-R antibody construct is multispecific.

In one embodiment of the invention, the antibody is conjugated to a chemotherapeutic agent.

In still another embodiment of the invention, the chemotherapeutic agent is an agent that disrupts DNA synthesis. In one embodiment, the agent that disrupts DNA synthesis is a nucleoside analog compound, including, for example, gemcitabine. In still another embodiment, the agent that disrupts DNA synthesis is an anthracycline compound, including, for example, adriamycin.

In still another embodiment of the invention, the chemotherapeutic agent is a topoisomerase I inhibitor, including, for example, Camptosar. In a further embodiment, the chemotherapeutic agent is an alkylating agent, including, for example, a platinum compound. In one embodiment, the platinum compound is either carboplatin and cisplatin.

In still another embodiment, the chemotherapeutic agent of the invention is a plant alkaloid. In one embodiment, said plant alkaloid is a taxane, including, for example, Taxol.

In one embodiment, a method for inhibiting tumor volume comprises administering an effective amount of a lymphotoxin-beta receptor (LT-β-R) agonist and an effective amount of a chemotherapeutic agent, which is not a lymphotoxin receptor agonist, wherein the administration of the LT-β-R agonist and the chemotherapeutic agent results in supra-additive inhibition of the tumor. The supra-additive inhibition of the tumor may be synergistic, and in certain embodiments, the supra-additive inhibition of the tumor has a combination index of less than 1.00. Alternatively the combination index is between about 0.85 to about 0.90; between about 0.70 to about 0.85; between about 0.30 to about 0.70; between about 0.10 to about 0.30. In yet another embodiment the combination index is less than 0.10. The supra-additive inhibition of the tumor may in other embodiments be potentiated, and in certain embodiments, the supra-additive inhibition of the tumor has a p-value of less than 0.05. Alternatively the supra-additive inhibition of the tumor has a p-value between about 0.05 to about 0.04; between about 0.04 to about 0.03; between about 0.03 to about 0.02; between about 0.02 to about 0.01. In yet another embodiment the p-value is less than 0.01.

Any of a variety of LT-β-R agonists may be used in the methods of the present invention. In certain embodiments, the LT-β-R agonist may be an anti-LT-β-R antibody. In one embodiment, the anti-LT-β-R antibody is a monoclonal antibody. In certain embodiments, the monoclonal antibody may be selected from the group consisting of: BKA11, CDH10, BCG6, AGH1, BDA8, CBE11 and BHA10. In other embodiments, the anti-LT-β-R antibody is a humanized antibody. In certain embodiments, the humanized antibody may be selected from the group consisting of: huCBE11 and huBHA10. In one embodiment, the humanized antibody is huCBE11. Humanized antibodies for use in the present invention may be produced in certain embodiments by a cell line selected from the group consisting of: E46.4 (ATCC patent deposit designation PTA-3357) or cell line E77.4 (ATCC patent deposit designation 3765). In still other embodiments, the anti-LT-β-R antibody is a multivalent anti-LT-β-R antibody construct, and in certain embodiments, may be multispecific. In one embodiment of the invention, the anti-LT-β-R antibody is conjugated to a chemotherapeutic agent.

Likewise, any of a variety of chemotherapeutic agents may be used in the methods of the invention, provided that the combination of the agonist and agent achieves inhibition of a tumor greater than that expected by the simple addition of the effects of the agonist and agent alone. In certain embodiments, the chemotherapeutic agent is an agent that disrupts DNA synthesis. In one embodiment, the agent that disrupts DNA synthesis is a nucleoside analog compound. In one embodiment, the nucleoside analog compound is gemcitabine. In another embodiment, the agent that disrupts DNA synthesis is an anthracycline compound, and in certain embodiments, the anthracycline compound is adriamycin. In other embodiments, the chemotherapeutic agent is a topoisomerase I inhibitor. In one embodiment, the topoisomerase I inhibitor is irinotecan, including, for example, Camptosar. The chemotherapeutic agent in other embodiments may be an alkylating agent. In one embodiment, the alkylating agent is a platinum compound, and in certain embodiments may be selected from the group consisting of carboplatin and cisplatin. In one embodiment, the platinum compound is cisplatin. In still other embodiments, the chemotherapeutic agent may be a plant alkaloid. In one embodiment, the plant alkaloid is a taxane, and in certain embodiments may be Taxol.

The present invention provides methods for screening for chemotherapeutic agents which have a supra-additive effect on inhibiting tumor volume when administered with a lymphotoxin-beta receptor (LT-β-R) agonist. In one embodiment, such a method comprises: (a) contacting a first tumor in a test subject with a LT-β-R agonist and measuring inhibition of tumor volume; (b) contacting a comparable second tumor in a test subject with a candidate chemotherapeutic agent and measuring inhibition of tumor volume; and (c) contacting a comparable third tumor in a test subject with both the LT-β-R agonist and the candidate chemotherapeutic agent and measuring inhibition of tumor volume; wherein, when the inhibition of tumor volume in the presence of both the LT-β-R agonist and the candidate chemotherapeutic agent is greater than the sum of the inhibition of tumor volume by each of the LT-β-R agonist and the candidate chemotherapeutic agent, the candidate chemotherapeutic agent is considered to have a supra-additive effect on inhibiting tumor volume.

Pharmaceutical compositions for use in the methods of the present invention are also provided. In one embodiment, a pharmaceutical composition comprises an effective amount of a LT-β-R agonist, an effective amount of a chemotherapeutic agent, which is nota LT-β-R agonist, and a pharmaceutically acceptable carrier, wherein the combined administration of the LT-β-R agonist and the chemotherapeutic agent results in supra-additive inhibition of a tumor. In certain embodiments, the chemotherapeutic agent is selected from the group consisting of: agents that disrupt DNA synthesis, nucleoside analog compounds, alkylating agents, and plant alkaloids. In certain embodiments, the LT-β-R agonist may be an anti-LT-β-R antibody, and may in some embodiments be a humanized antibody. In one embodiment, the humanized antibody may be huCBE11. In other embodiments, the anti-LT-β-R antibody may be a multivalent anti-LT-β-R antibody construct.

Furthermore pharmaceutical delivery devices for use in the methods are provided. In one embodiment, a pharmaceutical delivery device contains or is able to be loaded with an effective amount of a LT-β-R agonist, an effective amount of a chemotherapeutic agent, which is not a LT-β-R agonist, and a pharmaceutically acceptable carrier, wherein the administration of the LT-β-R agonist and the chemotherapeutic agent with said device results in supra-additive inhibition of a tumor. In certain embodiments, the administration of said agonist and said chemotherapeutic agent with said device is simultaneous. The agonist and chemotherapeutic agent may in certain embodiments be mixed in the device prior to administration with the device. In still other embodiments, the administration of the agonist and chemotherapeutic agent with the device is consecutive.

Methods of treating cancer or inhibiting tumor volume with the subject compositions and delivery devices are also provided. In one embodiment, a method of treating cancer in a subject comprises administering to the subject an effective amount of a pharmaceutical composition of the invention. In certain embodiments, the subject is human. In certain embodiments, the cancer comprises a solid tumor. The composition may be administered locally to the site of the tumor. In one embodiment, the composition is administered directly to the arterial blood supply of the tumor. In another embodiment, a method of treating cancer in a subject comprises administering to the subject an effective amount of a LT-β-R agonist and an effective amount of a chemotherapeutic agent, which is not a LT-β-R agonist with a pharmaceutical delivery device of the invention. In other embodiments, a method of inhibiting tumor volume in a subject comprises administering to the subject an effective amount of a composition of the invention. In still another embodiment, a method of inhibiting tumor volume in a subject comprises administering to the subject an effective amount of a LT-β-R agonist and an effective amount of a chemotherapeutic agent, which is not a LT-β-R agonist with a pharmaceutical delivery device of the invention.

The invention further provides kits including subject pharmaceutical compositions or drug delivery devices, and optionally instructions for their use. Uses for such kits include, for example, therapeutic applications. In certain embodiments, the subject compositions contained in any kit have been lyophilized and require rehydration before use.

In one embodiment, the instant invention provides a pharmaceutical delivery device containing or able to be loaded with: (1) an effective amount of a LT-β-R agonist; (2) an effective amount of at least one chemotherapeutic agent, which is not a LT-β-R agonist; and (3) a pharmaceutically acceptable carrier; such that the administration of the LT-β-R agonist and the chemotherapeutic agent with said device results in supra-additive inhibition of a tumor. In one embodiment, the device administers the LT-β-R agonist and chemotherapeutic agent simultaneously. In another embodiment, the LT-β-R agonist and chemotherapeutic agent are mixed in the device prior to simultaneous administration with the device. In a separate embodiment, the LT-β-R agonist and chemotherapeutic agent are administered consecutively with the device.

In other embodiments, cancer is treated in a subject by administering to the subject an effective amount of a LT-β-R agonist and an effective amount of a chemotherapeutic agent, which is not a LT-β-R agonist, with any of the the pharmaceutical delivery devices supra.

Another embodiment of the instant invention provides a method of treating cancer in a subject comprising administering to the subject an effective amount of a pharmaceutical composition of any of the pharmaceutical composition claims. In one embodiment, the subject is human. In another embodiment, the cancer comprises a solid tumor. For treatment of a solid tumor, one embodiment provides for local administration of the pharmaceutical composition to the site of the tumor. In another embodiment regarding treatment of a solid tumor, the pharmaceutical composition is administered directly to the arterial blood supply of the tumor.

In another embodiment of the instant invention, tumor volume is inhibited in a subject by administering to the subject an effective amount of any of the pharmaceutical compositions supra. In a separate embodiment, tumor volume is inhibited in a subject by administering to the subject an effective amount of a LT-β-R agonist and an effective amount of a chemotherapeutic agent, which is not a LT-β-R agonist, with any of the pharmaceutical delivery devices supra.

In still another embodiment, the instant invention provides a kit for treating cancer in a subject, comprising any of the pharmaceutical compositons supra. In another embodiment, the kit further comprises instructions for administering said composition to said subject.

In another embodiment, the instant invention provides a kit for treating cancer in a subject with a pharmaceutical delivery device, comprising an effective amount of a LT-β-R agonist and an effective amount of a chemotherapeutic agent, which is not a LT-β-R agonist and optionally instructions for use.

In a final embodiment, the invention provides a method of screening for chemotherapeutic agents which have a supra-additive effect on inhibiting tumor volume when administered with a lymphotoxin-beta receptor (LT-β-R) agonist comprising:
(a) contacting a first tumor in a test subject with a LT-β-R agonist and measuring inhibition of tumor volume;
(b) contacting a comparable second tumor in a test subject with a candidate chemotherapeutic agent and measuring inhibition of tumor volume; and
(c) contacting a comparable third tumor in a test subject with both the LT-β-R agonist and the candidate chemotherapeutic agent and measuring inhibition of tumor volume;

wherein, when the inhibition of tumor volume in the presence of both the LT-β-R agonist and the candidate chemotherapeutic agent is greater than the sum of the inhibition of tumor volume by each of the LT-β-R agonist and the candidate chemotherapeutic agent, the candidate chemotherapeutic agent is considered to have a supra-additive effect on inhibiting tumor volume.

Other features and advantages of the invention will be apparent from the following detailed description, and from the claims.

### BRIEF DESCRIPTION OF THE FIGURES

FIG. 1 depicts a graph showing the effect of irinotecan (Camptosar) in combination with huCBE11 (squares) against WiDr human colorectal adenocarcinoma tumor weight over the course of treatment, as compared to a saline control (crosses), irinotecan alone (circles), and huCBE11 alone (triangles). The first dose of each agent is indicated by an arrow.

FIG. 2 depicts a graph showing the effect of gemcitabine in combination with huCBE11 (squares) against WiDr human colorectal adenocarcinoma tumor weight over the course of treatment, as compared to a saline control (crosses), gemcitabine alone (circles), and huCBE11 alone (triangles). The first dose of each agent is indicated by an arrow.

FIG. 3 depicts a graph showing the effect of taxol in combination with huCBE11 (squares) against WiDr human colorectal adenocarcinoma tumor weight over the course of treatment, as compared to a saline control (crosses), taxol alone (circles), and huCBE11 alone (triangles). The first dose of each agent is indicated by an arrow.

FIG. 4 depicts a graph showing the effect of cisplatin (CDDP) in combination with huCBE11 (squares) against WiDr human colorectal adenocarcinoma tumor weight over the course of treatment, as compared to a saline control (crosses), cis-platin alone (circles), and huCBE11 alone (triangles). The first dose of each agent is indicated by an arrow.

FIG. 5 depicts a graph showing the effect of adriamycin in combination with huCBE11 (squares) against WiDr human colorectal adenocarcinoma tumor weight over the course of treatment, as compared to a saline control (crosses), adriamycin alone (circles), and huCBE11 alone (triangles). The first dose of each agent is indicated by an arrow.

FIG. 6 depicts a graph showing the effect of cisplatin (1 mg/kg) in combination with huCBE11 (triangles; 500 μg) against WiDr human colorectal adenocarcinoma tumor weight over the course of treatment, as compared to a saline control (crosses), cisplatin alone (filled squares), and huCBE11 alone (open squares). Dosings of each agent are indicated by arrows.

FIG. 7 depicts a graph showing the effect of adriamycin (6 mg/kg) in combination with huCBE11 (filled squares; 500 μg) against WiDr human colorectal adenocarcinoma tumor weight over the course of treatment, as compared to a saline control (filled triangles), adriamycin-alone (filled circles), and huCBE11 alone (open squares). Dosings of each agent are indicated by arrows.

FIG. 8 depicts a graph showing the effect of Camptosar (3 mg/kg) in combination with huCBE11 (diamonds; 20 mg/kg) against KM-20L2 human colorectal adenocarcinoma tumor weight over the course of treatment, as compared to a saline control (squares), Camptosar alone (triangles), and huCBE11 alone (circles). Dosings of each agent are indicated by arrows.

FIG. 9 shows a plot of the combination index at each effect level for the combination of huCBE11 and Camptosar at decreasing tumor volume, in the WiDr adrenocarcinoma model. The combination index (CI) was plotted against the fraction affected (Fa). A combination index of <1 indicates synergy.

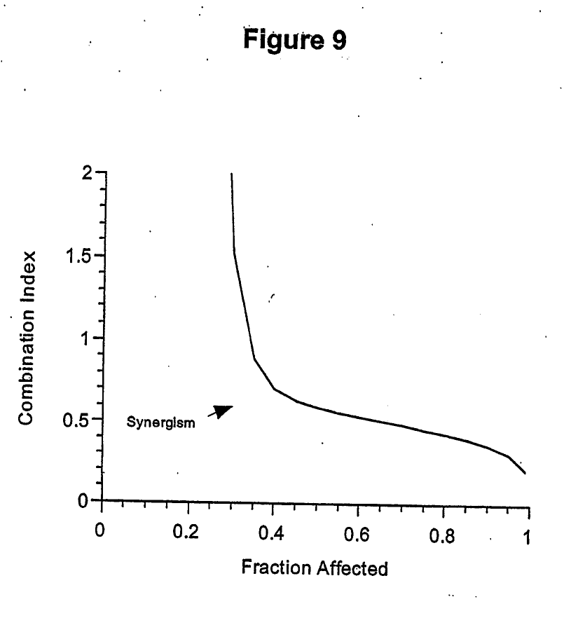

FIG. 10 shows plots of the combination index at each effect level for the combination of huCBE11 and Camptosar (Fixed dose ratio of 1:0.63 huCBE11:Camptosar) at decreasing tumor volume, across multiple time points of treatment in the KM-20L2 adrenocarcinoma model. The combination index (CI) was plotted against the percent of tumor suppression observed. A combination index of <1 indicates synergy.

FIG. 11 shows a plot of the combination index at each effect level for the combination of huCBE11 and gemcitabine at decreasing tumor volume, in the WiDr adrenocarcinoma model. The combination index (CT) was plotted against the fraction affected (Fa). A combination index of <1 indicates synergy.

FIG. 12 depicts a graph showing the effect of gemcitabine (20 mg/kg) in combination with huCBE11 (squares; 4 mg/kg) against KM-20L2 human colorectal adenocarcinoma tumor weight over the course of treatment, as compared to a saline control (crosses), gemcitabine alone (circles), and huCBE11 alone (triangles). Dosings of each agent are indicated by arrows.

FIG. 13 shows plots of the combination index at each effect level for the combination of huCBE11 and gemcitabine (Fixed dose ratio of 4:5 huCBE11:gemcitabine) at decreasing tumor volume, across multiple time points of treatment in the KM-20L2 adrenocarcinoma model. The combination index (CI) was plotted against the percent of tumor suppression observed. A combination index of <1 indicates synergy.

FIG. 14 depicts three-dimensional graphs of dose-response ranges for huCBE11:gemcitabine combined treatment, when administered at a fixed ratio of 4:5 to KM-20L2 adrenocarcinoma model mice.

FIG. 15 shows a plot of the combination index at each effect level for the combination of huCBE11 and Taxol at decreasing tumor volume. The combination index (CI) was plotted against the fraction affected (Fa). A combination index of <1 indicates synergy.

### DETAILED DESCRIPTION OF THE INVENTION

1. Definitions

For convenience, before further description of the present invention, certain terms employed in the specification, examples and appended claims are defined here.

The singular forms “a”, “an”, and “the” include plural references unless the context clearly dictates otherwise.

The term “administering” includes any method of delivery of a pharmaceutical composition or therapeutic agent into a subject's system or to a particular region in or on a subject. The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration. “Parenteral administration” and “administered parenterally” means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrasternal injection and infusion.

The term “agent that disrupts DNA synthesis” refers to any molecule or compound able to reduce or inhibit the process of DNA synthesis. Examples of agents that disrupt DNA synthesis include but are not limited to inhibitors of enzymes which effect or promote DNA synthesis, such as topoisomerase I, or nucleoside analogs such as pyrimidine or purine analogs.

The term “alkylating agent” refers to any molecule or compound able to react with the nucleophilic groups of (for examples, amines, alcohols, phenols, organic and inorganic acids) and thus add alkyl groups (for example, ethyl or methyl groups) to another molecule such as a protein or nucleic acid. Examples of alkylating agents used as chemotherapeutic agents include busulfan, chloarmbucil, cyclophosphamide, ifosfamide, mechlorethamine, melphalan, thiotepa, various nitrosourea compounds, and platinum compounds such as cisplatin and carboplatin.

The term “anti-tumor activity” refers to the ability of a substance or composition to block the proliferation of, or to induce the death of tumor cells which interact with that substance or composition. The term “apoptosis” refers to a process of programmed cell death.

The term “cancer” or “neoplasia” refers in general to any malignant neoplasm or spontaneous growth or proliferation of cells. The term as used herein encompasses both fully developed malignant neoplasms, as well as premalignant lesions. A subject having “cancer”, for example, may have a tumor or a white blood cell proliferation such as leukemia. In certain embodiments, a subject having cancer is a subject having a tumor, such as a solid tumor. Cancers involving a solid tumor include but are not limited to non small cell lung cancer (NSCLC), testicular cancer, lung cancer, ovarian cancer, uterine cancer, cervical cancer, pancreatic cancer, colorectal cancer (CRC), breast cancer, as well as on prostate, gastric, skin, stomach, esophagus and bladder cancer.

The term “chemotherapeutic agent” refers to any molecule or composition used to treat disease caused by a foreign cell or malignant cell, such as a tumor cell. Chemotherapeutic agents contemplated herewith include agents that can be conjugated to the antibodies of the present invention or alternatively agents that can be used in combination with the antibodies of the present invention without being conjugated to the antibody. In one embodiment of the invention, chemotherapeutic agents which can be used in combination with the antibodies of the invention include, but are not limited to the following: platinums (i.e. cis platinum), anthracyclines, nucleoside analogs (purine and pyrimidine), taxanes, camptothecins, epipodophyllotoxins, DNA alkylating agents, folate antagonists, vinca alkaloids, ribonucleotide reductase inhibitors, estrogen inhibitors, progesterone inhibitors, androgen inhibitors, aromatase inhibitors, interferons, interleukins, monoclonal antibodies, taxol, camptosar, adriamycin (dox), 5-FU and gemcitabine. Such chemotherapeutics may be employed in the practice of the invention in combination with the antibodies of the invention by coadministration of the antibody and the chemotherapeutic. In one embodiment, the antibodies of the invention are nonconjugated to a chemotherapeutic agent. In another embodiment of the invention, the chemotherapeutic agent and the anti-LT-βR agonist antibody are conjugated.

The term “combination index” refers to a measure of the combined dose-effect of at least two molecules or compounds as determined by the method of Chou and Talalay (1984) Adv. Enz. Regul. 22: 27, which is further described in the Detailed Description of the Invention and Examples. If a dose effect is synergistic, the combination index is less than 1.00. Alternatively the combination index showing synergism may be between about 0.85 to about 0.90; between about 0.70 to about 0.85; between about 0.30 to about 0.70; between about 0.10 to about 0.30.

The term “effective amount” refers to that amount of a compound, material, or composition comprising a compound of the present invention which is sufficient to effect a desired result, including, but not limited to, for example, reducing tumor volume either in vitro or in vivo. An effective amount of a pharmaceutical composition of the present invention is an amount of the pharmaceutical composition that is sufficient to effect a desired clinical result, including but not limited to, for example, ameliorating, stabilizing, preventing or delaying the development of cancer in a patient. In either case, an effective amount of the compounds of the present invention can be administered in one or more administrations. Detection and measurement of these above indicators are known to those of skill in the art, including, but not limited for example, reduction in tumor burden, inhibition of tumor size, reduction in proliferation of secondary tumors, expression of genes in tumor tissue, presence of biomarkers, lymph node involvement, histologic grade, and nuclear grade.

The term “humanized antibody” refers to an antibody or antibody construct in which the complementarity determining regions (CDRs) of an antibody from one species have been grafted onto the framework regions of the variable region of a human

The term “inhibition of tumor volume” refers to any reduction or decrease in tumor volume. The ability of a pharmaceutical composition or therapeutic agent to inhibit tumor volume may be measured by the “fraction affected value”. The term “fraction affected value (Fa)” refers to a measure of the fraction inhibition of tumor value, calculated by dividing the treatment group mean tumor volume decrease by the control group mean tumor volume. An Fa of 1.000 indicates complete inhibition of the tumor. The calculation of Fa is further described in the Detailed Description of the Invention.

The term “lymphotoxin-beta receptor (LT-β-R) agonist” refers to any agent which can augment ligand binding to the LT-β-R, cell surface LT-β-R clustering and/or LT-β-R signaling.

The term “anti-LT-β-R antibody” refers to any molecule that recognizes and binds to at least one epitope of the LT-beta receptor. Examples of anti-LT-β-R antibodies include monoclonal antibodies, chimeric antibodies, humanized antibodies and multivalent antibodies. “Antibody” is intended to include whole antibodies, e.g., of any isotype (IgG, IgA, IgM, IgE, etc.), and includes fragments thereof which are also specifically reactive with a vertebrate, e.g., mammalian, protein, as well as fusion proteins comprising a fragment of an antibody. Antibodies may be fragmented using conventional techniques and the fragments screened for utility in the same manner as described above for whole antibodies. Thus, the term includes segments of proteolytically-cleaved or recombinantly-prepared portions of an antibody molecule that are capable of selectively reacting with a certain protein. Non-limiting examples of such proteolytic and/or recombinant fragments include Fab, F(ab′)2, Fab′, Fv, and single chain antibodies (sFv) containing a V[L] and/or V[H] domain joined by a peptide linker. The term “antibody” also includes “antibody constructs”, which may comprise two or more variable regions attached to a constant region from any one of the five Ig classes (for example IgA, IgD, IgE, IgG and IgM). The subject invention includes polyclonal, monoclonal, humanized, or other purified preparations of antibodies and recombinant antibodies.

The term “monoclonal antibody” refers to an antibody molecule that contains only one species of an antigen-binding site capable of immunoreacting with or binding to a particular epitope. For preparation of monoclonal antibodies directed an epitope, or derivatives, fragments, analogs or homologs thereof, any technique that provides for the production of antibody molecules by continuous cell line culture may be utilized. Such techniques include, but are not limited to, the hybridoma technique (see Kohler & Milstein (1975) Nature 256:495-497); the trioma technique; the human B-cell hybridoma technique (see Kozbor, et al. (1983) Immunol. Today 4:72) and the EBV hybridoma technique to produce human monoclonal antibodies (see Cole, et al., 1985 In: Monoclonal Antibodies and Cancer Therapy Alan R. Liss, Inc., pp. 77-96). Human monoclonal antibodies may be utilized in the practice of the present invention and may be produced by using human hybridomas (see Cote et al. (1983). Proc. Natl. Acad. Sci. USA 80: 2026-2030) or by transforming human B-cells with Epstein Barr Virus in vitro (see Cole et al. (1985) In: Monoclonal Antibodies and Cancer Therapy, Alan R. Liss, Inc., pp. 77-96).

The phrase “multivalent antibody” or “multivalent antibody construct” refers to an antibody or antibody construct comprising more than one antigen recognition site. For example, a “bivalent” antibody construct has two antigen recognition sites, whereas a “tetravalent” antibody construct has four antigen recognition sites. The terms “monospecific”, “bispecific”, “trispecific”, “tetraspecific”, etc. refer to the number of different antigen recognition site specificities (as opposed to the number of antigen recognition sites) present in a multivalent antibody construct of the invention. For example, a “monospecific” antibody construct's antigen recognition sites all bind the same epitope. A “bispecific” antibody construct has at least one antigen recognition site that binds a first epitope and at least one antigen recognition site that binds a second epitope that is different from the first epitope. A “multivalent monospecific” antibody construct has multiple antigen recognition sites that all bind the same epitope. A “multivalent bispecific” antibody construct has multiple antigen recognition sites, some number of which bind a first epitope and some number of which bind a second epitope that is different from the first epitope. Examples of such multivalent antibody constructs, and methods of making and using the same, are described in the Provisional Patent Application entitled, “Anti-LT-β-R Multispecific Multivalent Antibody Constructs, and Methods of Making and Using the Same”, filed Dec. 20, 2002, U.S. Provisional Application 60/435154, which is hereby incorporated by reference in its entirety.

The term “P-value” refers to the probability value. The p-value indicates how likely it is that the result obtained by the experiment is due to chance alone. In one embodiment of the invention, the p-value of the Two-Tailed One-Sample T-Test. A p-value of less than 0.05 is considered statistically significant, that is, not likely to be due to chance alone. Alternatively a statistically significant p-value may be between about 0.05 to about 0.04; between about 0.04 to about 0.03; between about 0.03 to about 0.02; between about 0.02 to about 0.01. In certan cases, the p-value may be less than 0.01. As used herein, the p-value is used to measure whether or not there is any statistically significant supra-additive inhibition of tumor volume when a lymphotoxin-beta receptor (LT-β-R) agonist and a chemotherapeutic agent, which is not a lymphotoxin receptor agonist, are administered to a tumor or subject having a tumor. There is biological relevance to the p-value when statistical significance is observed over a series of treatment days rather tha the occasional one day.

A “patient” or “subject” or “host” refers to either a human or non-human animal.

The term “pharmaceutical delivery device” refers to any device that may be used to administer a therapeutic agent or agents to a subject. Non-limiting examples of pharmaceutical delivery devices include hypodermic syringes, multichamber syringes, stents, catheters, transcutaneous patches, microneedles, microabraders, and implantable controlled release devices. In one embodiment, the term “pharmaceutical delivery device” refers to a dual-chambered syringe capable of mixing two compounds prior to injection.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

“Pharmaceutically-acceptable salts” refers to the relatively non-toxic, inorganic and organic acid addition salts of compounds. These salts can be prepared in situ in the administration vehicle or the dosage form manufacturing process, or by separately reacting a purified compound of the invention in its free base form with a suitable organic or inorganic acid, and isolating the salt thus formed during subsequent purification. The pharmaceutically acceptable salts of the subject compounds include the conventional nontoxic salts or quaternary ammonium salts of the compounds, e.g., from non-toxic organic or inorganic acids.

The term “plant alkaloid” refers a compound belonging to a family of alkaline, nitrogen-containing molecules derived from plants that are biologically active and cytotoxic. Examples of plant alkoids include, but are not limited to, taxanes such as docetaxel and paclitaxel and vincas such as vinblastine, vincristine, and vinorelbine. In one embodiment, the plant alkaloid is Taxol.

The term “supra-additive” refers to an effect from a combination of agents, wherein the total effect from the combination of the agents is greater than the sum of the effects due to each of the individual agents. Examples of supra-additive effects include potentiation and synergy. The term “potentiation” refers to a case in which simultaneous effect of two or more agents is greater than the sum of the independent effects of the agents. In one embodiment, potentiation occurs when one agent has no inhibitory effect when administered alone, but potentiates the effect of a second agent when administered in combination. In one embodiment of the invention, only one of the LT-β-R agonist or chemotherapeutic agent individually has the ability to inhibit tumor volume, but in combination the effect of the agents is potentiated.

The term “supra-additive inhibition of a tumor” refers a total decrease in tumor volume which is greater than the sum of the effects of a combination of agents due to each individual agent. In one embodiment of the invention, supra-additive inhibition of a tumor includes a mean tumor inhibition produced by administration of a combination of a LT-β-R agonist and a chemotherapeutic agent, which is not a LT-β-R agonist, that is statistically signficantly higher than the sum of the tumor inhibition produced by the individual administration of either a LT-β-R agonist or chemotherapeutic agent alone. Whether tumor inhibition produced by combination administration of a LT-β-R agonist and a chemotherapeutic agent is “statistically significantly higher” than the expected additive value of the individual compounds may be determined by a variety of statistical methods as described in the Detailed Description of the Invention.

The term “synergistic” refers to a combination which is more effective than the additive effects of any two or more single agents. In one embodiment of the invention, the term synergistic includes a type of supra-additive inhibition in which both the LT-β-R agonist and chemotherapeutic agent individually have the ability to inhibit tumor volume.

The term “topoisomerase I inhibitor” refers to a molecule or compound that inhibits or reduces the biological activity of a topoisomerase I enzyme. Non-limiting examples of topoisomerase I inhibitors include anthracyclines such as daunombicin, doxorabicin, and idambicin and epipodophyllotoxins such as etoposide and teniposide.

“Treating cancer” or “treating a subject having cancer” refers to administering to a subject to a pharmaceutical treatment, e.g., the administration of a drug, such that the extent of cancer is decreased or prevented. Treating cancer means to inhibit the replication of cancer cells, to inhibit the spread of cancer, to decrease tumor size, to lessen or reduce the number of cancerous cells in the body, and/or to ameliorate or alleviate the symptoms of the disease caused by the cancer. The treatment is considered therapeutic if there is a decrease in mortality and/or morbidity. In one embodiment of the invention, the term treating cancer refers to decreasing tumor size. Treatment includes (but is not limited to) administration of a composition, such as a pharmaceutical composition, and may be performed either prophylactically, or subsequent to the initiation of a pathologic event.

The term “tumor volume” refers to the total size of the tumor, which includes the tumor itself plus affected lymph nodes if applicable. Tumor volume may be determined by a variety of methods known in the art, such as, e.g. by measuring the dimensions of the tumor using calipers, computed tomography (CT) or magnetic resonance imaging (MRI) scans, and calculating the volume using equations based on, for example, the z-axis diameter, or on standard shapes such as the sphere, ellipsoid, or cube.

2. Lymphotoxin-[3-Receptor (LT-(3-R) Agonists

Any of a variety of LT-β-R agonists may be used in the methods of the present invention. U.S. Pat. No. 6,312,691 and WO 96/22788, the contents of which are hereby incorporated in their entirety, describe methods and compositions for the treatment of cancer using LT-β-R agonists to trigger cancer cell death. For example, U.S. Pat. No. 6,312,691 describes LT-β-R agonists for use in the invention including membrane-bound LT-alpha/beta complexes, soluble LT-alpha/beta complexes and anti-LT-β-R antibodies and methods for their preparation and purification.

The surface LT-alpha/beta heteromeric complex can be reconstructed by co-transfection of host cells with both the LT-alpha and LT-beta genes. Surface LT complexes cannot be observed on stable cell lines which express either LT gene alone. However, if the host cell normally produces large amounts of LT-alpha (e.g. RPMI 1788 cells; see below), then transfection with a LT-beta gene with encodes the desired LT-beta polypeptide should be sufficient to generate LT-alpha/beta complexes comprising full-length LT-alpha subunits.

Co-expression of LT-alpha and LT-beta polypeptides in a number of eukaryotic expression systems leads to their assembly and export as active ligand (Crowe et al., J. Immunol. Methods, 168, 79-89 (1994)). Host systems that can be used include but are not limited to CHO cells, COS cells, B cells including myelomas, baculovirus-infected insect cells and yeast. The LT-alpha subunit of the LT-alpha/beta heteromeric complexes of this invention can be selected from lymphotoxin-alpha, native human or animal lymphotoxin-alpha, recombinant lymphotoxin-alpha, soluble lymphotoxin-alpha, secreted lymphotoxin-alpha, lymphotoxin-alpha muteins having LT-alpha biological activity, or lymphotoxin-alpha fragments of any of the above having LT-alpha biological activity.

Soluble (non-membrane-bound) LT-alpha/beta heteromeric complexes comprise LT-beta subunits which have been changed form a membrane-bound to a soluble form. These complexes are described in detail in applicants' co-pending international application (PCT/US93/11669, published Jan. 9, 1992 as WO 94/13808). Soluble LT-beta peptides are defined by the amino acid sequence of lymphotoxin-beta wherein the sequence is cleaved at any point between the end of the transmembrane region (i.e. at about amino acid #44) and the first TNF homology region (i.e. at amino acid #88) according to the numbering system of Browning et al. (1993) Cell 72:847.

Soluble LT-beta polypeptides may be produced by truncating the N-terminus of LT-beta to remove the cytoplasmic tail and transmembrane region (Crow et al., Science, 264, pp. 707-710 (1994). Alternatively, the transmembrane domain may be inactivated by deletion, or by substitution of the normally hydrophobic amino acid residues which comprise a transmembrane domain with hydrophilic ones. In either case, a substantially hydrophilic hydropathy profile is created which will reduce lipid affinity and improve aqueous solubility. Deletion of the transmembrane domain is preferred over substitution with hydrophilic amino acid residues because it avoids introducing potentially immunogenic epitopes.

Soluble LT-alpha/beta heteromeric complexes may be produced by co-transfecting a suitable host cell with DNA encoding LT-alpha and soluble LT-beta (Crow et al., (1994) J. Immunol. Methods, 168:79). Soluble LT-beta secreted in the absence of LT-alpha is highly oligomerized. However, when co-expressed with LT-alpha, a 70 kDa trimeric-like structure is formed which contains both proteins. It is also possible to produce soluble LT-alphal/beta2 heteromeric complexes by transfecting a cell line which normally expresses only LT-alpha (such as the RPMI 1788 cells discussed above) with a gene encoding a soluble LT-beta polypeptide. LT-alpha and LT-beta polypeptides may be separately synthesized, denatured using mild detergents, mixed together and renatured by removing the detergent to form mixed LT heteromeric complexes which can be separated (see below).

In certain embodiments, the LT-β-R agonist may be an anti-LT-β-R antibody. In certain embodiments, the anti-LT-β-R antibody may be a polyclonal antibody. Following immunization, antisera reactive with LT-β-R may be obtained and, if desired, polyclonal antibodies isolated from the serum. In another embodiment, the anti-LT-β-R antibody is a monoclonal antibody. In certain embodiments, the monoclonal antibody may be selected from the group consisting of: BKA11, CDH10, BCG6, AGH1, BDA8, CBE11 and BHA10. To produce monoclonal antibodies, antibody producing cells (lymphocytes) may be harvested from an immunized animal and fused by standard somatic cell fusion procedures with immortalizing cells such as myeloma cells to yield hybridoma cells. Such techniques are well known in the art, an include, for example, the hybridoma technique (originally developed by Kohler and Milstein, (1975) Nature, 256: 495-497), as the human B cell hybridoma technique (Kozbar et al., (1983) Immunology Today, 4: 72), and the EBV-hybridoma technique to produce human monoclonal antibodies (Cole et al., (1985) Monoclonal Antibodies and Cancer Therapy, Alan R. Liss, Inc. pp. 77-96). Hybridoma cells can be screened immunochemically for production of antibodies specifically reactive with LT-β-R and the monoclonal antibodies isolated. Monoclonal antibodies for use in the present invention may be produced in certain embodiments by a cell line selected from the group consisting of the cells lines in Table 1:

TABLE 1

| CELL LINE | mAb Name | Accession No. |
|---|---|---|
| a) AG.H1.5.1 | AGH1 | HB 11796 |
| b) BD.A8.AB9 | BDA8 | HB 11798 |
| c) BC.G6.AF5 | BCG6 | B 11794 |
| d) BH.A10 | BHA10 | B 11795 |
| e) BK.A11.AC10 | BKA11 | B 11799 |
| f) CB.E11.1 | CBE11 | B 11793 |
| g) CD.H10.1 | CDH10 | B 11797 |

In other embodiments, the anti-LT-β-R antibody is a humanized antibody. In certain embodiments, the humanized antibody may be selected from the group consisting of: huCBE11 and huBHA10. In one embodiment, the humanized antibody is huCBE11, as described in PCT publication no WO 02/30986; U.S. Provisional Appln. No. 60/240,285; U.S. provisional appln. No. 60/275,289; U.S. provisional appln. No. 60/299,987. In another embodiment, the humanized antibody is huBHA10, as described in PCT application no. PCT US03/20762; U.S. provisional appln. No. 60/392,993; and U.S. provisional appln. No. 60/417,372.

Applicants' applications in the above table, the contents of which are hereby incorporated in their entirety, describe methods and compositions for the treatment of cancer using huCBE11 and huBHA10, respectively, to trigger cancer cell death. Animals are immunized with the desired antigen, the corresponding antibodies are isolated, and the portion of the variable region sequences responsible for specific antigen binding are removed. The animal-derived antigen binding regions are then cloned into the appropriate position of human antibody genes in which the antigen binding regions have been deleted. See, e.g. Jones, P. et al. (1986), Nature 321, 522-525 or Tempest et al. (1991) Biotechnology 9, 266-273. Also, transgenic mice, or other mammals, may be used to express humanized antibodies. Such humanization may be partial or complete. Humanized antibodies minimize the use of heterologous (inter-species) sequences in human antibodies, and are less likely to elicit immune responses in the treated subject. Humanized antibodies for use in the present invention may be produced in certain embodiments by a cell line selected from the group consisting of: E46.4 (ATCC patent deposit designation PTA-3357) or cell line E77.4 (ATCC patent deposit designation 3765).

Various forms of anti-LT-β-R antibodies can also be made using standard recombinant DNA techniques (Winter and Milstein, Nature, 349, pp. 293-99 (1991)). For example, “chimeric” antibodies can be constructed in which the antigen binding domain from an animal antibody is linked to a human constant domain (e.g. Cabilly et al., U.S. Pat. No. 4,816,567; Morrison et al., Proc. Natl. Acad. Sci. U.S.A., 81, pp. 6851-55 (1984)). Chimeric antibodies reduce the observed immunogenic responses elicited by animal antibodies when used in human clinical treatments. Construction of different classes of recombinant anti-LT-β-R antibodies can also be accomplished by making chimeric or humanized antibodies comprising the anti-LT-β-R variable domains and human constant domains (CH1, CH2, CH3) isolated from different classes of immunoglobulins. For example, anti-LT-beta-R IgM antibodies with increased antigen binding site valencies can be recombinantly produced by cloning the antigen binding site into vectors carrying the human mu. chain constant regions (Arulanandam et al., J. Exp. Med., 177, pp. 1439-50 (1993); Lane et al., Eur. J. Immunol., 22, pp. 2573-78 (1993); Traunecker et al., Nature, 339, pp. 68-70 (1989)). In addition, standard recombinant DNA techniques can be used to alter the binding affinities of recombinant antibodies with their antigens by altering amino acid residues in the vicinity of the antigen binding sites. See, e.g. (Queen et al., Proc. Natl. Acad. Sci. U.S.A., 86, pp. 10029-33 (1989); WO 94/04679).

Anti-LT-β-R antibodies of the invention may also be cross-linked, as known in the art. The final conjugate after cross-linking is preferably soluble in physiological fluids such as blood. The polymer should not be highly immunogenic in the conjugate form, and should possess a viscosity compatible with intravenous infusion or injection if either is an intended route of administration.

In still other embodiments, the anti-LT-β-R antibody is a multivalent anti-LT-β-R antibody construct, and in certain embodiments, may be multispecific. Examples of such multivalent antibody constructs, and methods of making and using the same, are described in the U.S. provisional appln. No. 60/435,154 and PCT Appln. No. PCT/US2003/041393 entitled “Anti-LT-β-R Multispecific Multivalent Antibody Constructs, and Methods of Making and Using the Same”, which is hereby incorporated by reference in its entirety.

In one embodiment, the multivalent antibody are agonists of the lymphotoxin-beta receptor and comprise at least two domains that are capable of binding to the receptor and inducing LT-β-R signaling. These constructs can include a heavy chain containing two or more variable regions comprising antigen recognitions sites specific for binding the LT-beta receptor and a light chain containing one or more variable regions or can be constructed to comprise only heavy chains or light chains containing two or more variable regions comprising CDRs specific for binding the LT-beta receptor.

In one aspect, the present invention includes multivalent antibody constructs that are human lymphotoxin-beta receptor (LT-β-R) agonists. In one embodiment, a multivalent antibody construct comprises at least one antigen recognition site specific for a LT-β-R epitope. In certain embodiments, at least one of the antigen recognition sites is located within a scFv domain, while in other embodiments, all antigen recognition sites are located within scFv domains.

Antibody constructs may be bivalent, trivalent, tetravalent or pentavalent. In certain embodiments, the antibody construct is monospecific. In one embodiment, the antibody construct is specific for the epitope to which CBE11 binds. In other embodiments, the antibody of the invention is a monospecific tetravalent LT-β-R agonist antibody comprising four CBE11-antigen recognition sites. In another embodiment, the antibody construct is specific for the BHA10 epitope, and, in some embodiments, is tetravalent. In any of these embodiments, at least one antigen recognition site may be located on a scFv domain, and in certain of these embodiments, all antigen recognition sites may be located on scFv domains. Antibodies may be multispecific, wherein the antibody of the invention binds to different epitopes on human LT-β receptors.

In certain embodiments, the antibody construct is bispecific. In other embodiments, the antibody construct is specific for at least two members of the group of lymphotoxin-beta receptor (LT-β-R) epitopes consisting of the epitopes to which one of following antibodies bind: BKA11, CDH10, BCG6, AGH1, BDA8, CBE11 and BHA10. In one embodiment, the antibody construct is specific for the epitope to which the CBE11 and BHA10 antibodies bind, and in certain embodiments, is tetravalent. In one embodiment, the antibody construct has two CBE11-specific antigen recognition sites and two BHA10-specific recognition sites, wherein the antibody is a bispecific tetravalent LT-β-R agonist antibody. In any of the multispecific antibody constructs, at least one antigen recognition site may be located on a scFv domain, and in certain embodiments, all antigen recognition sites are located on scFv domains.

In still other embodiments, the antibody constructs of the invention comprise the following polynucleotide sequences and encoded polypeptide sequences of Table 2:

TABLE 2

| Sequence | Description |
|---|---|
| SEQ ID NO: 1 | Polynucleotide sequence of mature heavy |
| | chain of the hCBE11/hBHA10 Bispecific-1 |
| | antibody construct |
| SEQ ID NO: 2 | Polypeptide sequence of mature heavy chain |
| | of the hCBE11/hBHA10 Bispecific-1 |
| | antibody construct |
| SEQ ID NO: 3 | Polynucleotide sequence of mature light |
| | chain of the hCBE11/hBHA10 Bispecific-1 |
| | antibody construct |
| SEQ ID NO: 4 | Polypeptide sequence of mature light chain |
| | of the hCBE11/hBHA10 Bispecific-1 |
| | antibody construct |
| SEQ ID NO: 5 | Polynucleotide sequence of mature |
| | hCBE11/hBHA10 Bispecific-2 antibody |
| | construct |
| SEQ ID NO: 6 | Polypeptide sequence of mature |
| | hCBE11/hBHA10 Bispecific-2 antibody |
| | construct |
| SEQ ID NO: 7 | Polynucleotide sequence of mature heavy |
| | chain of the hCBE11 Monospecific-1 |
| | antibody construct |
| SEQ ID NO: 8 | Polypeptide sequence of mature heavy chain |
| | of the hCBE11 Monospecific-1 antibody |
| | construct. |
| SEQ ID NO: 9 | Polynucleotide sequence of mature hCBE11 |
| | Monospecific-2 antibody construct |
| SEQ ID NO: 10 | Polypeptide sequence of mature hCBE11 |
| | Monospecific-2 antibody construct |
| SEQ ID NO: 11 | Polynucleotide sequence of mature CBE11 |
| | pentameric heavy chain antibody construct |
| SEQ ID NO: 12 | Polypeptide sequence of mature CBE11 |
| | pentameric heavy chain antibody construct |
| SEQ ID NO: 13 | Polynucleotide sequence of mature CBE11 |
| | chimeric light chain antibody construct |
| SEQ ID NO: 14 | Polypeptide sequence of mature CBE11 |
| | chimeric light chain antibody construct |

Pentameric CBE11 constructs comprising the heavy and light chains described in SEQ ID NOs: 11-14 can also be used in screening assays used to identify combination therapies.

The antigen recognition sites or entire variable regions may be derived from one or more parental antibodies. The parental antibodies can include naturally occurring antibodies or antibody fragments, antibodies or antibody fragments adapted from naturally occurring antibodies, antibodies constructed de novo using sequences of antibodies or antibody fragments known to be specific for the LT-beta receptor. Sequences that may be derived from parental antibodies include heavy and/or light chain variable regions and/or CDRs, framework regions or other portions thereof.

Multivalent, multispecific antibodies may contain a heavy chain comprising two or more variable regions and/or a light chain comprising one or more variable regions wherein at least two of the variable regions recognize different epitopes on the LT-beta receptor.

Methods for making multivalent multispecific antibodies are known in the art. Traditional production of full length bispecific antibodies is based on the coexpression of two immunoglobulin heavy chain-light chain pairs, where the two chains have different specificities (Milstein et al., Nature, 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J., 10:3655-3659 (1991).

Multivalent, anti-LT-β-R antibodies may be constructed in a variety different ways using a variety of different sequences derived from parental anti-LT-β-R antibodies, including murine or humanized BHA10 (Browning et al., J. Immunol. 154: 33 (1995); Browning et al. J. Exp. Med. 183:867 (1996)) and/or murine or humanized CBE11 (U.S. Pat. No. 6,312,691).

According to a different approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy-chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

Another embodiment of the invention includes the use of human anti-LT-β-R antibodies, which can be produced in nonhuman animals, such as transgenic animals harboring one or more human immunoglobulin transgenes. Such animals may be used as a source for splenocytes for producing hybridomas, as is described in U.S. Pat. No. 5,569,825, WO00076310, WO00058499 and WO00037504 and incorporated by reference herein.

In some embodiments, the antibodies and antibody fragments of the invention may be chemically modified to provide a desired effect. For example, pegylation of antibodies and antibody fragments of the invention may be carried out by any of the pegylation reactions known in the art, as described, for example, in the following references: Focus on Growth Factors 3:4-10 (1992); EP 0 154 316; and EP 0 401 384 (each of which is incorporated by reference herein in its entirety). Preferably, the pegylation is carried out via an acylation reaction or an alkylation reaction with a reactive polyethylene glycol molecule (or an analogous reactive water-soluble polymer). A preferred water-soluble polymer for pegylation of the antibodies and antibody fragments of the invention is polyethylene glycol (PEG). As used herein, “polyethylene glycol” is meant to encompass any of the forms of PEG that have been used to derivatize other proteins, such as mono (Cl—ClO) alkoxy- or aryloxy-polyethylene glycol.

Methods for preparing pegylated antibodies and antibody fragments of the invention will generally comprise the steps of (a) reacting the antibody or antibody fragment with polyethylene glycol, such as a reactive ester or aldehyde derivative of PEG, under conditions whereby the antibody or antibody fragment becomes attached to one or more PEG groups, and (b) obtaining the reaction products. It will be apparent to one of ordinary skill in the art to select the optimal reaction conditions or the acylation reactions based on known parameters and the desired result.

Pegylated antibodies and antibody fragments may generally be used to treat conditions that may be alleviated or modulated by administration of the antibodies and antibody fragments described herein. Generally the pegylated antibodies and antibody fragments have increased half-life, as compared to the nonpegylated antibodies and antibody fragments. The pegylated antibodies and antibody fragments may be employed alone, together, or in combination with other pharmaceutical compositions.

In other embodiments of the invention the antibodies or antigen-binding fragments thereof are conjugated to albumen using art recognized techniques.

In another embodiment of the invention, antibodies, or fragments thereof, are modified to reduce or eliminate potential glycosylation sites. Such modified antibodies are often referred to as “aglycosylated” antibodies. In order to improve the binding affinity of an antibody or antigen-binding fragment thereof, glycosylation sites of the antibody can be altered, for example, by mutagenesis (e.g., site-directed mutagenesis). “Glycosylation sites” refer to amino acid residues which are recognized by a eukaryotic cell as locations for the attachment of sugar residues. The amino acids where carbohydrate, such as oligosaccharide, is attached are typically asparagine (N-linkage), serine (O-linkage), and threonine (O-linkage) residues. In order to identify potential glycosylation sites within an antibody or antigen-binding fragment, the sequence of the antibody is examined, for example, by using publicly available databases such as the website provided by the Center for Biological Sequence Analysis (see http://www.cbs.dtu.dk/services/NetNGlyc/ for predicting N-linked glycoslyation sites) and http://www.cbs.dtu.dk/services/NetOGlyc/ for predicting O-linked glycoslyation sites). Additional methods for altering glycosylation sites of antibodies are described in U.S. Pat. Nos. 6,350,861 and 5,714,350.

In yet another embodiment of the invention, antibodies or fragments thereof can be altered wherein the constant region of the antibody is modified to reduce at least one constant region-mediated biological effector function relative to an unmodified antibody. To modify an antibody of the invention such that it exhibits reduced binding to the Fc receptor (FcR), the immunoglobulin constant region segment of the antibody can be mutated at particular regions necessary for FcR interactions (see e.g., Canfield et al (1991) J. Exp. Med. 173:1483; and Lund, J. et al. (1991) J. of Immunol. 147:2657). Reduction in FcR binding ability of the antibody may also reduce other effector functions which rely on FcR interactions, such as opsonization and phagocytosis and antigen-dependent cellular cytotoxicity.

In a particular embodiment the invention further features antibodies having altered effector function, such as the ability to bind effector molecules, for example, complement or a receptor on an effector cell. In particular, the humanized antibodies of the invention have an altered constant region, e.g., Fc region, wherein at least one amino acid residue in the Fc region has been replaced with a different residue or side chain thereby reducing the ability of the antibody to bind the FcR. Reduction in FcR binding ability of the antibody may also reduce other effector functions which rely on FcR interactions, such as opsonization and phagocytosis and antigen-dependent cellular cytotoxicity. In one embodiment, the modified humanized antibody is of the IgG class, comprises at least one amino acid residue replacement in the Fc region such that the humanized antibody has an altered effector function, e.g., as compared with an unmodified humanized antibody. In particular embodiments, the humanized antibody of the invention has an altered effector function such that it is less immunogenic (e.g., does not provoke undesired effector cell activity, lysis, or complement binding), and/or has a more desirable half-life while retaining specificity for LTβR or a ligand thereof.

Alternatively, the invention features humanized antibodies having altered constant regions to enhance FcR binding, e.g., FcγR3 binding. Such antibodies are useful for modulating effector cell function, e.g., for increasing ADCC activity, e.g., particularly for use in oncology applications of the invention.

As used herein, “antibody-dependent cell-mediated cytotoxicity” and “ADCC” refer to a cell-mediated reaction in which nonspecific cytotoxic cells that express FcRs (e.g. Natural Killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRII and FcγRIII. of the antibody, e.g., a conjugate of the antibody and another agent or antibody.

In still another embodiment, the anti-LT-β-R antibodies of the invention can be conjugated to a chemotherapeutic agent to inhibit tumor volume in a supra-additive manner. Exemplary chemotherapeutics that can be conjugated to the antibodies of the present invention include, but are not limited to radioconjugates (90Y, 131I, 99mTc, 111In, 186Rh, et al.), tumor-activated prodrugs (maytansinoids, CC-1065 analogs, clicheamicin derivatives, anthracyclines, vinca alkaloids, et al.), ricin, diptheria toxin, pseudomonas exotoxin.

It is envisioned that other LT-β-R agonists—including but not limited to those identified using in vitro tumor cell cytotoxicity assays—may have similar anti-tumor effects in vivo when administered either alone or in combination to animals or humans.

The cytotoxic effects of LT-β-R agonists on tumor cells may be enhanced by the presence of a LT-β-R activating agent, particularly IFN-gamma. Any agent which is capable of inducing interferons, preferably IFN-gamma, and which potentiates the cytotoxic effects of LT-alpha/beta heteromeric complexes and anti-LT-β-R antibodies on tumor cells falls within the group of LT-β-R agonists of this invention. For example, clinical experiments have demonstrated interferon induction by double stranded RNA (dsRNA) treatment. Accordingly, polyriboguanylic/polyribocytidylic acid (poly-rG/rC) and other forms of dsRNA are effective as interferon inducers (Juraskova et al., Eur. J. Pharmacol., 221, pp. 107-11 (1992)).

The LT-β-R agonists produced as described above may be purified to a suitable purity for use as a pharmaceutical composition. Generally, a purified composition will have one species that comprises more than about 85 percent of all species present in the composition, more than about 85%, 90%, 95%, 99% or more of all species present. The object species may be purified to essential homogeneity (contaminant species cannot be detected in the composition by conventional detection methods) wherein the composition consists essentially of a single species. A skilled artisan may purify a polypeptide of the invention using standard techniques for protein purification in light of the teachings herein. Purity of a polypeptide may be determined by a number of methods known to those of skill in the art, including for example, amino-terminal amino acid sequence analysis, gel electrophoresis and mass-spectrometry analysis.

3. Supra-Additive Inhibition of LT-PR Agonists and Chemotherapeutic Agents

3.1 Chemotherapeutic Agents

The invention provides for the use of a lymphotoxin-beta receptor agonist in combination with a chemotherapeutic agent to treat cancer. Likewise, any of a variety of chemotherapeutic agents may be used or tested for use in the methods of the invention, provided that the combination of the agonist and agent acheives inhibition of a tumor greater than that expected by the simple addition of the effects of the agonist and agent alone. Chemotherapy drugs are divided into several categories based on how they affect specific chemical substances within cancer cells, which cellular activities or processes the drug interferes with, and which specific phases of the cell cycle the drug affects.

In certain embodiments, the chemotherapeutic agent is an agent that disrupts DNA synthesis. In one embodiment, the agent that disrupts DNA synthesis is a nucleoside analog compound. In certain embodiments, the nucleoside analog compound is gemcitabine. In another embodiment, the agent that disrupts DNA synthesis is a anthracycline compound, and in certain embodiments, the anthracycline compound is adriamycin.

In other embodiments, the chemotherapeutic agent is a topoisomerase I inhibitor. In certain embodiments, the topoisomerase I inhibitor is Camptosar.

The chemotherapeutic agent in other embodiments is an alkylating agent. Alkylating agents work directly on DNA to prevent the cancer cell from reproducing. As a class of drugs, these agents are not phase-specific (in other words, they work in all phases of the cell cycle). Alkylating agents are commonly active against chronic leukemias, non-Hodgkin's lymphoma, Hodgkin's disease, multiple myeloma, and certain cancers of the lung, breast, and ovary. Examples of alkylating agents include busulfan, cisplatin, carboplatin, chlorambucil, cyclophosphamide, ifosfamide, dacarbazine (DTIC), mechlorethamine (nitrogen mustard), and melphalan. In one embodiment, the alkylating agent is a platinum compound, and in certain embodiments may be selected from the group consisting of carboplatin and cisplatin. In certain embodiments, the platinum compound is cisplatin.

In still other embodiments, the chemotherapeutic agent is a plant alkaloid. In one embodiment, the plant alkaloid is a taxane, including, for example, Taxol.

Various forms of the chemotherapeutic agents and/or other biologically active agents may be used. These include, without limitation, such forms as uncharged molecules, molecular complexes, salts, ethers, esters, amides, and the like, which are biologically activated when implanted, injected or otherwise inserted into the tumor.

3.2 Screening for Supra-Additive Chemotherapeutic Agents

The present invention provides methods for screening for chemotherapeutic agents which have a supra-additive effect on inhibiting tumor volume when administered with a lymphotoxin-beta receptor (LT-β-R) agonist. In one embodiment, such a method comprises: (a) contacting a first tumor in a test subject with a LT-β-R agonist and measuring inhibition of tumor volume; (b) contacting a comparable second tumor in a test subject with a candidate chemotherapeutic agent and measuring inhibition of tumor volume; and (c) contacting a comparable third tumor in a test subject with both the LT-β-R agonist and the candidate chemotherapeutic agent and measuring inhibition of tumor volume; wherein, when the inhibition of tumor volume in the presence of both the LT-β-R agonist and the candidate chemotherapeutic agent is greater than the sum of the inhibition of tumor volume by each of the LT-β-R agonist and the candidate chemotherapeutic agent, the candidate chemotherapeutic agent is considered to have a supra-additive effect on inhibiting tumor volume.

As used herein, “supra-additive inhibition of a tumor” refers to mean tumor inhibition produced by administration of a combination of a LT-β-R agonist and a chemotherapeutic agent that is statistically signficantly higher than the sum of the tumor inhibition produced by the individual administration of either a LT-β-R agonist or chemotherapeutic agent alone. Whether tumor inhibition produced by combination administration of a LT-β-R agonist and a chemotherapeutic agent is “statistically significantly higher” than the expected additive value of the individual compounds may be determined by as follows. Such supra-additive inhibition may be potentiated, or synergistic, as defined above.

In general, potentiation may be assessed by determining whether the combination treatment produces a mean tumor volume decrease in a treatment group that is statistically significantly supra-additive when compared to the sum of the mean tumor volume decreases produced by the individual treatments in their treatment groups respectively. The mean tumor volume decrease may be calculated as the difference between control group and treatment group mean tumor volume. The fractional inhibition of tumor volume, “fraction affected” (Fa), may be calculated by dividing the treatment group mean tumor volume decrease by control group mean tumor volume. An Fa of 1.000 indicates complete inhibition of the tumor. Testing for statistically significant potentiation requires the calculation of Fa for each treatment group. The expected additive Fa for a combination treatment was taken to be the sum of mean Fas from groups receiving either element of the combination. The Two-Tailed One-Sample T-Test, for example, may be used to evaluate how likely it is that the result obtained by the experiment is due to chance alone, as measured by the p-value. A p-value of less than 0.05 is considered statistically significant, including but not limited to between about 0.05 to about 0.04; between about 0.04 to about 0.03; between about0.03 to about 0.02; between about 0.02 to about 0.01., that is, not likely to be due to chance alone. In certain cases, the p-value may be less than 0.01. Thus, Fa for the combination treatment group must be statistically significantly higher than the expected additive Fa for the single element treatment groups to deem the combination as resulting in a potentiated supra-additive effect.

Whether or not a synergistic effect results from a combination treatment may be evalued by the median-effect/combination-index isobologram method (Chou, T., and Talalay, P. (1984) Ad. Enzyme Reg. 22:27-55). In this method, combination index (CI) values are calculated for different dose-effect levels based on parameters dervied from median-effect plots of the LT-β-R agonist alone, the chemotherapeutic agent alone, and the combination of the two at fixed molar ratios. CI values of <1 indicate synergy, including but not limited to between about 0.85 to about 0.90; between about 0.70 to about 0.85; between about 0.30 to about 0.70; between about 0.10 to about 0.30. In yet another embodiment the combination index is less than 0.10. This analysis may beperformed using CalcuSyn, Windows® Software for Dose Effect Analysis (Biosoft, Cambridge UK).

Any method known or later developed in the art for analyzing whether or not a supra-additive effect exists for a combination therapy is contemplated for use in screening for suitable chemotherapeutic agents.

In one embodiment of the invention, LT-β-R agonist/chemotherapeutic agent combinations which have a combined supra-additive effect at treating cancer are identified through screening assays known in the art, including assays which examine inhibition of tumor volume. Tumor volume is commonly used as a proxy for assessing the anti-cancer efficacy of a compound or combination of compounds (see for example, Naundorf, et al. (2002) Int. J. Cancer, 100:101; Goel., et al. (2001) Clin Cancer Res. 7: 175; Liao, et al. (2000) Cancer Res. 60:6805; Prewett, et al. (1999) Cancer Res. 59: 5209; Boudreau M. D., et al. (2001) Cancer Res. 61: 1386). Tumor volume can be studied using xenograft models. Examples of xenograft models used to screen potential agonist/chemotherapeutic agents include WiDr human coloractal adenocarcinoma and KM-20L2 human coloractal adenocarcinoma. A decrease in or inhibition of tumor volume using a murine model has also been described for the anti-Erb2 antibody Herceptin (see U.S. Pat. No. 6,627,196) and anti-VEGF antibodies (see U.S. Pat. No. 5,955,311). Guidelines for assessment of tumor size (e.g. tumor volume) are presented in, “NCI—cooperative group—industry relationship guidelines, appendix XVII (Status of the NCI preclinical antitumor agent discovery screen, principles and practice of oncology updates)”.

Other methods of evaluating the anti-cancer efficacy of an antibody and/or chemotherapeutic compound(s) include analysis of survival and mortality and molecular marker evaluation when appropriate (e.g. PSA in prostate cancer, TPA in colon cancer), wherein levels of such markers may be evaluated in evaluating anti-cancer activity of a compound.

4. Pharmaceutical Compositions And Delivery Devices

4.1 Pharmaceutical Compositions

The invention provides pharmaceutical compositions comprising the above-described LT-β-R agonist and chemotherapeutic agents. In one aspect, the present invention provides pharmaceutically acceptable compositions which comprise a therapeutically-effective amount of one or more of the compounds described above, formulated together with one or more pharmaceutically acceptable carriers (additives) and/or diluents. In another aspect, certain embodiments, the compounds of the invention can be administered as such or in admixtures with pharmaceutically acceptable carriers and can also be administered in conjunction with other chemotherapeutic agents. Conjunctive (combination) therapy thus includes sequential, simultaneous and separate, or co-administration of the active compound in a way that the therapeutical effects of the first administered one is not entirely disappeared when the subsequent is administered.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art. While it is possible for a compound of the present invention to be administered alone, it is preferable to administer the compound as a pharmaceutical formulation (composition). The compounds according to the invention may be formulated for administration in any convenient way for use in human or veterinary medicine, by analogy with other pharmaceuticals.

As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally. Inone embodiment, the pharmaceutical compositions are formulated for parenteral administration. In another embodiment, the pharmaceutical composition is formulated for intraarterial injection. In another embodiment, the pharmaceutical compositions are formulated for systemic administration.

In other cases, the compounds of the present invention may contain one or more acidic functional groups and, thus, are capable of forming pharmaceutically-acceptable salts with pharmaceutically-acceptable bases.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuiming and preservative agents. Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol, glycerol monostearate, and non-ionic surfactants; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent. The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required. The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof. Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents. These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

4.2 Delivery methods and devices

The pharmaceutical compositions of this invention may also be administered using a variety of pharmaceutical delivery devices may, which may include hypodermic syringes, multichamber syringes, stents, catheters, transcutaneous patches, microneedles, microabraders, and implantable controlled release devices. In one embodiment, a pharmaceutical delivery device contains or is able to be loaded with at least an effective amount of a LT-β-R agonist and an effective amount of a chemotherapeutic agent. The device may in some embodiments be able to deliver or administer the LT-β-R agonist and chemotherapeutic agent simultaneously. The device may have the ability to mix the agonist and chemotherapeutic agent prior to administration with the device. In still other embodiments, the device may be able to administer the agonist and chemotherapeutic agent consecutively.

One potential pharmaceutical delivery device is a multi-chambered syringe capable of mixing two compounds prior to injection, or delivering them sequentially. A typical dual-chamber syringe and a process for automated manufacture of prefilled such syringes is disclosed in Neue Verpackung, No.3, 1988, p. 50-52; Drugs Made in Germany, Vol. 30, Pag. 136-140 (1987); Pharm. Ind. 46, Nr. 10 (1984) p.1045-1048 and Pharm. Ind. 46, Nr. 3 (1984) p. 317-318. The syringe type ampoule is a dual chamber device with a front bottle type opening for needle attachment, two pistons and an exterior type by-pass for mixing a lyophilized powder in the front chamber with a reconstitution liquid in the rear chamber. The process described includes the main steps of washing and siliconizing the syringe barrels, insertion of multiple barrels in carrier trays, sterilization, introduction of middle piston through barrel rear end, turning the trays upside down, introduction of the powder solution through the front opening, lyophilization to dry powder, closure of front opening while in the lyophilizing chamber, turning of trays, introduction of the reconstitution liquid through barrel rear end, insertion of rear piston, removal of products from trays and final control and packaging. Ampoules prefilled with the various components may be manufactured for use with the syringes.

In another embodiment, the multichamber syringe is a Lyo-ject system (Vetter Pharma Turm, Yardley, Pa.). The Lyo-Ject allows the user to lyophilize the drug directly in a syringe, which is packaged with the diluent for quick reconstitution and injection. It is described in U.S. Pat. Nos. 4,874,381 and 5,080,649.

In other embodiments, the compounds are administered using two separate syringes, catheters, microneedles, or other device capable of accomplishing injection.

The pharmaceutical compositions of this invention may also be administered using microspheres, liposomes, other microparticulate delivery systems or sustained release formulations placed in, near, or otherwise in communication with affected tissues or the bloodstream. Suitable examples of sustained release carriers include semipermeable polymer matrices in the form of shaped articles such as suppositories or microcapsules. Implantable or microcapsular sustained release matrices include polylactides (U.S. Pat. No. 3,773,319; EP 58,481), copolymers of L-glutamic acid and gamma ethyl-L-glutamate (Sidman et al., Biopolymers, 22, pp. 547-56 (1985)); poly(2-hydroxyethyl-methacrylate) or ethylene vinyl acetate (Langer et al., J. Biomed. Mater. Res., 15, pp. 167-277 (1981); Langer, Chem. Tech., 12, pp. 98-105 (1982)). The compositions of this invention will be administered at an effective dose to treat the particular clinical condition addressed. Determination of a preferred pharmaceutical formulation and a therapeutically efficient dose regiment for a given application is well within the skill of the art taking into consideration, for example, the condition and weight of the patient, the extent of desired treatment and the tolerance of the patient for the treatment.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

5. Therapeutic Methods

The present invention further provides novel therapeutic methods of treating cancer comprising administering to the subject an effective amount of a subject pharmaceutical composition, optionally using a delivery device described above.

The methods of the present invention may be used to treat any cancer, including but not limited to treating solid tumors, Examples of solid tumors that can be treated by compounds of the present invention, include but are not limited to breast, testicular, lung, ovary, uterine, cervical, pancreatic, non small cell lung (NSCLC), colon, as well as on prostate, gastric, skin, stomach, esophagus and bladder cancer In certain embodiments, the method comprises parenterally administering an effective amount of a subject pharmaceutical composition to a subject. In one embodiment, the method comprises intraarterial administration of a subject composition to a subject. In other embodiments, the method comprises administering an effective amount of a subject composition directly to the arterial blood supply of a tumor in a subject. In one embodiment, the methods comprises administering an effective amount of a subject composition directly to the arterial blood supply of the cancerous tumor using a catheter. In embodiments where a catheter is used to administer a subject composition, the insertion of the catheter may be guided or observed by fluoroscopy or other method known in the art by which catheter insertion may be observed and/or guided. In another embodiment, the method comprises chemoembolization. For example a chemoembolization method may comprise blocking a vessel feeding the cancerous tumor with a composition comprised of a resin-like material mixed with an oil base (e.g., polyvinyl alcohol in Ethiodol) and one or more chemotherapeutic agents. In still other embodiments, the method comprises systemic administration of a subject composition to a subject.

In general, chemoembolization or direct intraarterial or intravenous injection therapy utilizing pharmaceutical compositions of the present invention is typically performed in a similar manner, regardless of the site. Briefly, angiography (a road map of the blood vessels), or more specifically in certain embodiments, arteriography, of the area to be embolized may be first performed by injecting radiopaque contrast through a catheter inserted into an artery or vein (depending on the site to be embolized or injected) as an X-ray is taken. The catheter may be inserted either percutaneously or by surgery. The blood vessel may be then embolized by refluxing pharmaceutical compositions of the present invention through the catheter, until flow is observed to cease. Occlusion may be confirmed by repeating the angiogram. In embodiments where direct injection is used, the blood vessel is then infused with a pharmaceutical composition of the invention in the desired dose.

Embolization therapy generally results in the distribution of compositions containing inhibitors throughout the interstices of the tumor or vascular mass to be treated. The physical bulk of the embolic particles clogging the arterial lumen results in the occlusion of the blood supply. In addition to this effect, the presence of an anti-angiogenic factor(s) prevents the formation of new blood vessels to supply the tumor or vascular mass, enhancing the devitalizing effect of cutting off the blood supply. Direct intrarterial or intravenous generally results in distribution of compositions containing inhibitors throughout the interstices of the tumor or vascular mass to be treated as well. However, the blood supply is not generally expected to become occluded with this method.

Within one aspect of the present invention, primary and secondary tumors of the liver or other tissues may be treated utilizing embolization or direct intraarterial or intravenous injection therapy. Briefly, a catheter is inserted via the femoral or brachial artery and advanced into the hepatic artery by steering it through the arterial system under fluoroscopic guidance. The catheter is advanced into the hepatic arterial tree as far as necessary to allow complete blockage of the blood vessels supplying the tumor(s), while sparing as many of the arterial branches supplying normal structures as possible. Ideally this will be a segmental branch of the hepatic artery, but it could be that the entire hepatic artery distal to the origin of the gastroduodenal artery, or even multiple separate arteries, will need to be blocked depending on the extent of tumor and its individual blood supply. Once the desired catheter position is achieved, the artery is embolized by injecting compositions (as described above) through the arterial catheter until flow in the artery to be blocked ceases, preferably even after observation for 5 minutes. Occlusion of the artery may be confirmed by injecting radio-opaque contrast through the catheter and demonstrating by fluoroscopy or X-ray film that the vessel which previously filled with contrast no longer does so. In embodiments where direct injection is used, the artery is infused by injecting compositions (as described above) through the arterial catheter in a desired dose. The same procedure may be repeated with each feeding artery to be occluded.

In most embodiments, the subject pharmaceutical compositions will incorporate the substance or substances to be delivered in an amount sufficient to deliver to a patient a therapeutically effective amount of an incorporated therapeutic agent or other material as part of a prophylactic or therapeutic treatment. The desired concentration of active compound in the particle will depend on absorption, inactivation, and excretion rates of the drug as well as the delivery rate of the compound. It is to be noted that dosage values may also vary with the severity of the condition to be alleviated. It is to be further understood that for any particular subject, specific dosage regimens should be adjusted over time according to the individual need and the professional judgment of the person administering or supervising the administration of the compositions. Typically, dosing will be determined using techniques known to one skilled in the art. The selected dosage level will depend upon a variety of factors including the activity of the particular compound of the present invention employed, or the ester, salt or amide thereof, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

Dosage may be based on the amount of the composition per kg body weight of the patient. Other amounts will be known to those of skill in the art and readily determined. Alternatively, the dosage of the subject invention may be determined by reference to the plasma concentrations of the composition. For example, the maximum plasma concentration (Cmax) and the area under the plasma concentration-time curve from time 0 to infinity (AUC (0-4)) may be used. Dosages for the present invention include those that produce the above values for Cmax and AUC (0-4) and other dosages resulting in larger or smaller values for those parameters.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above.

The precise time of administration and amount of any particular compound that will yield the most effective treatment in a given patient will depend upon the activity, pharmacokinetics, and bioavailability of a particular compound, physiological condition of the patient (including age, sex, disease type and stage, general physical condition, responsiveness to a given dosage and type of medication), route of administration, and the like. The guidelines presented herein may be used to optimize the treatment, e.g., determining the optimum time and/or amount of administration, which will require no more than routine experimentation consisting of monitoring the subject and adjusting the dosage and/or timing.

While the subject is being treated, the health of the patient may be monitored by measuring one or more of the relevant indices at predetermined times during a 24-hour period. Treatment, including supplement, amounts, times of administration and formulation, may be optimized according to the results of such monitoring. The patient may be periodically reevaluated to determine the extent of improvement by measuring the same parameters, the first such reevaluation typically occurring at the end of four weeks from the onset of therapy, and subsequent reevaluations occurring every four to eight weeks during therapy and then every three months thereafter. Therapy may continue for several months or even years, with a minimum of one month being a typical length of therapy for humans. Adjustments to the amount(s) of agent administered and possibly to the time of administration may be made based on these reevaluations.

Treatment may be initiated with smaller dosages which are less than the optimum dose of the compound. Thereafter, the dosage may be increased by small increments until the optimum therapeutic effect is attained.

Knowing this helps oncologists decide which drugs are likely to work well together and, if more than one drug will be used, plan exactly when each of the drugs should be given (in which order and how often).

The combined use of several compounds of the present invention, or alternatively other chemotherapeutic agents, may reduce the required dosage for any individual component because the onset and duration of effect of the different components may be complimentary. In such combined therapy, the different active agents may be delivered together or separately, and simultaneously or at different times within the day. Toxicity and therapeutic efficacy of subject compounds may be determined by standard pharmaceutical procedures in cell cultures or experimental animals, e.g., for determining the LD50 and the ED50. Compositions that exhibit large therapeutic indices are preferred. Although compounds that exhibit toxic side effects may be used, care should be taken to design a delivery system that targets the compounds to the desired site in order to reduce side effects.

The data obtained from the cell culture assays and animal studies may be used in formulating a range of dosage for use in humans. The dosage of any supplement, or alternatively of any components therein, lies preferably within a range of circulating concentrations that include the ED50 with little or no toxicity. The dosage may vary within this range depending upon the dosage form employed and the route of administration utilized. For agents of the present invention, the therapeutically effective dose may be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information may be used to more accurately determine useful doses in humans. Levels in plasma may be measured, for example, by high performance liquid chromatography.

6. Kits

The present invention provides kits for treating various cancers. For example, a kit may comprise one or more pharmaceutical composition as described above and optionally instructions for their use. In still other embodiments, the invention provides kits comprising one more more pharmaceutical composition and one or more devices for accomplishing administration of such compositions. For example, a subject kit may comprise a pharmaceutical composition and catheter for accomplishing direct intraarterial injection of the composition into a cancerous tumor. In other embodiments, a subject kit may comprise pre-filled ampoules of an LT-β-R agonist and a chemotherapeutic agent, optionally formulated as a pharmaceutical, or lyophilized, for use with a delivery device.

### EXAMPLES

The present invention is further illustrated by the following examples which should not be construed as limiting in any way.

Materials and Methods

WiDr Mouse Model

In order to study the effects of chemotherapeutic agents in combination with huCBE11, the WiDr xenograft model was used. CBE11 has been shown to exhibit antitumor activity against WiDr tumors grown as xenografts in mice with severe combined immunodeficiency (SCID) (Browning et al. (1996) J. Exp. Med. 183:867). Therapeutic agents, i.e. LTβR agonist and chemotherapeutic agents, were administered to athymic nude mice who had been implanted with WiDr tumor cells. Antitumor activity, including any synergistic or potentiating effects of the combination therapy, was studied according to the growth of WiDr xenograft human colorectal tumors, wherein treatment was initiated on an established, preformed tumor mass.

WiDr cells were obtained from the American Type Culture Collection (Manassas, Va.). Cells were grown in vitro in 90% Eagle's Minimum Essential Medium with 2 mM L-glutamine and Earle's Balanced Salt Solution (BSS) adjusted to contain 1.5 g/L sodium bicarbonate, 0.1 mM non-essential amino acids, and 1 mM sodium pyruvate plus 10% fetal bovine serum (FBS) without antibiotics (5% CO₂). Bacterial cultures were performed on aliquots of the tumor homogenate preparation that was implanted into the mice to ensure that all cultures were negative for bacterial contamination at both 24 and 48 hours post implant.

An inoculum of 2×10⁶ WiDr cells in 200 μL RPMI 1640 without serum was implanted subcutaneously into the right flank area on Day 0. Tumor weight and body weight measurements were recorded twice-weekly beginning on Day 3, and also on Day 4 for studies including Camptosar. When the tumors measured approximately 5 mm in length by 5 mm in width, mice were randomized to treatment and control groups. Body weight measurements were recorded twice-weekly beginning on Day 0.

KM-20L2 Mouse Model

In order to study the effects of chemotherapeutic agents in combination with huCBE11, the KM-20L2 xenograft model was used. Therapeutic agents, i.e. LTβR agonist and chemotherapeutic agents, were administered to athymic nude mice who had been implanted with WiDr tumor cells. Antitumor activity, including any synergistic or potentiating effects of the combination therapy, was studied according to the growth of WiDr xenograft human colorectal tumors, wherein treatment was initiated on an established, preformed tumor mass.

KM-20L2 were obtained from the from the NCI tumor repository. Cells were grown in 90% RPMI-1640 with 10% fetal bovine serum without antibiotics. Bacterial cultures were performed on aliquots of the tumor cell homogenate preparation that were implanted into the mice to ensure that all cultures were negative for bacterial contamination at both 24 and 48 hours post implant.

An inoculum of 2×10⁶ or 3×10⁶ KM-20L2 cells in medium without serum was implanted subcutaneously into the right flank area of the mouse on Day 0. Tumor size measurements were recorded regularly. When the tumors measured approximately 5 mm in length by 5 mm in width (65 mg), mice were randomized into treatment and control groups.

Tumor Measurements

Tumor measurements were determined using Vernier calipers. Tumor size measurements were recorded regularly according to the study, until the termination of the study. The formula to calculate volume for a prolate ellipsoid was used to estimate tumor volume (mm³) from 2-dimensional tumor measurements: tumor volume (mm³)=(length×width² [L×W²])÷2. Assuming unit density, volume was converted to weight (i.e., 1 mm³=1 mg). Tumor growth inhibition was assessed as % T/C, where T is the mean tumor weight of the treatment group and C is the mean tumor weight of the control group. A % T/C value of 42% or less for this type of study is considered indicative of meaningful activity by the National Cancer Institute (USA). Animals were sacrificed accordingly.

Statistical Analysis

Statistical analysis of the tumor weight measurements was performed according to standard statistical methods. Mean, standard deviation (SD), and standard error of the mean (SEM) were determined for body weight and tumor weight for all dose groups at all assessments. Student's t test was performed on mean tumor weights at each assessment, including at the end of each study, to determine whether there were any statistically significant differences between each treatment group and the vehicle control group and between each combination treatment group and the respective huCBE11 group.

Analyses were performed to determine whether synergistic or potentiating antitumor activity occurred during combination treatment with huCBE11 and the chemotherapeutic agent. If treatment of mice bearing the WiDr tumor with the chemotherapeutic agent alone produced dose-responsive antitumor efficacy, synergism of the huCBE11 plus chemotherapeutic agent combinations could be formally assessed by calculating the Combination Index (Chou and Talalay (1984) Adv. Enz. Regu.22:27). In addition, potentiation by some combination treatments was assessed by determining whether combination treatment produced efficacy that was statistically significantly supra-additive when compared to the sum of efficacy produced by the individual treatments.

Antitumor efficacy was determined by comparing each treatment group's tumor volume with the control group's tumor volume. Mean tumor volume decrease was calculated as the difference between the control group and the treatment group mean tumor volume. The fractional inhibition of tumor volume, i.e., the fraction affected (Fa), was calculated by dividing the treatment group mean tumor volume decrease by the control group mean tumor volume. An Fa of 1.000 indicated complete inhibition of the tumor. Further statistical analysis was performed accordingly.

Synergy Analysis

Overall interpretation of the degree of synergism or antagonism(expressed as symbols) indicated by the CI is are described below in Table 3.

TABLE 3
Interpretation of symbols for describing synergism or antagonism

| Range of | | |
|---|---|---|
| Combination | | |
| Index | Symbol | Interpretation |
| <0.1 | +++++ | Very strong synergism |
| 0.1-0.3 | ++++ | Strong synergism |
| 0.3-0.7 | +++ | Synergism |
| 0.7-0.85 | ++ | Moderate synergism |
| 0.85-0.90 | + | Slight synergism |
| 0.90-1.10 | ± | Nearly additive |
| 1.10-1.20 | − | Slight antagonism |
| 1.20-1.45 | − − | Moderate antagonism |
| 1.45-3.3 | − − − | Antagonism |
| 3.3-10 | − − − − | Strong antagonism |
| >10 | − − − − − | Very strong antagonsim |

### Example 1

### Antitumor Efficacy of LTβR Agonist in Combination with Alkylating Chemotherapeutic Agent

Antitumor Efficacy of Combination of huCBE11 with Cisplatin

In order to determine whether administration of an alkylating chemotherapeutic agent, e.g., cisplatin, in combination with huCBE11 has supra-additive, antitumor activity, e.g., syngergistic or potentiating activity, cisplatin was administered in combination with huCBE11 using the WiDr xenograft model.

A dosing range study was performed to determine the appropriate cisplatin and huCBE11 dose(s) for studying the antitumor effects of cisplatin and huCBE11. The dosing study also examined the antitumor efficacy of each agent at inhibiting tumor growth individually. Athymic nude mice bearing established WiDr tumors were treated with a either saline (control), huCBE11 (50 μg or 500 μg), or cisplatin (doses ranging from 0.25 mg/kg to 2 mg/kg ) (saline control n=30; experimental groups n=10 per dose). Tumor size was measured on day 3 and regularly thereafter up to the staging day.

Tumor growth in the 2 mg/kg, 1 mg/kg, and 0.25 mg/kg cisplatin dose groups did not differ significantly from the saline control group at day 50. It was determined that cisplatin at 2 mg/kg to 0.25 mg/kg was inactive in the WiDr model based on the NCI activity criteria (% T/C of 42 or less). On Day 50, cisplatin produced a significant inhibition of WiDr human colorectal tumor growth in nude mice only at a dose of 0.5 mg/kg (P<0.05). In parallel studies, it was determined that on day 44, huCBE11 produced a significant inhibition of tumor growth at doses of 500 μg (P<0.001) and 50 μg (P<0.01). Treatment with cisplatin alone did not produce dose-responsive antitumor efficacy, thus synergism of the combination of cisplatin plus huCBE11 could not be assessed.

In order to determine whether the combination treatment of cisplatin and huCBE11 had a significant increase in inhibiting tumor growth, a combination study was performed on athymic nude mice bearing established WiDr tumor cells with established tumors as described above. This study compared the effect of huCBE11 (50 μg or 500 μg) and cisplatin (1 mg/kg and 2 mg/kg) in various combinations to determine efficacy, synergism, and potentiation. Four different combinations of doses of cisplatin (1 and 2 mg/kg) and huCBE11 (50 and 500 μg) were assessed.

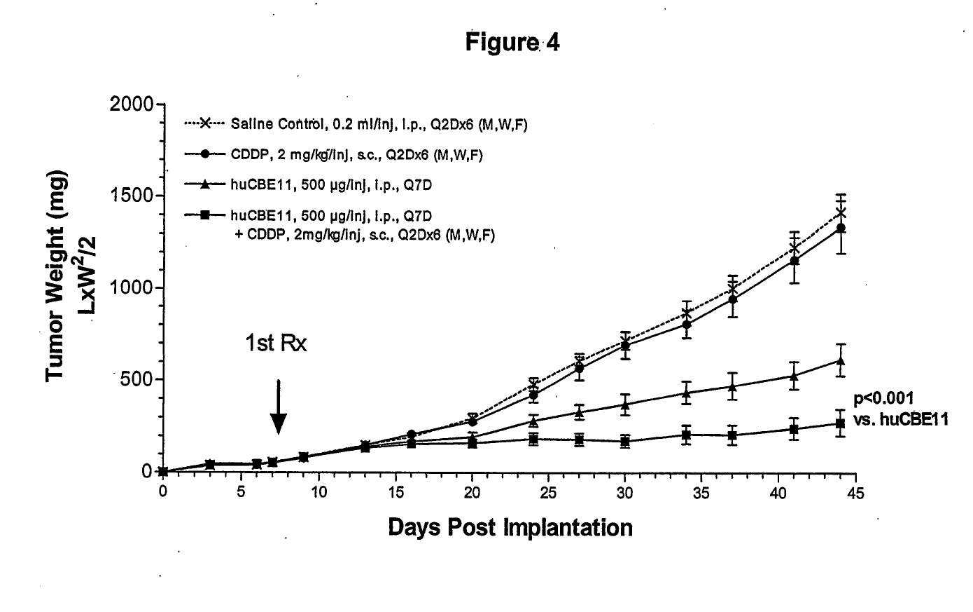

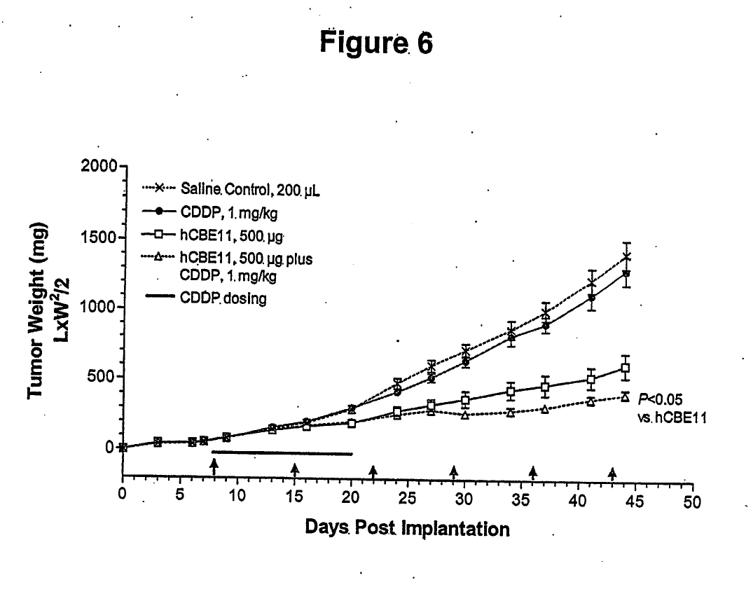

Results from the combination studies (shown in Tables 4-6 and FIGS. 4 and 6) demonstrate that huCBE11 in combination with cisplatin significantly decreases tumor volume in treated mice. All of the tumor data were taken at day 44. Antitumor efficacy was determined by comparing each treatment group's tumor volume with the control group's tumor volume. An Fa of 1.000 indicates complete inhibition of the tumor. Table 4 shows the dose-effect relationships for separate and combination treatments of huCBE11 and cisplatin.

TABLE 4
Dose effect relationships

| | | | | | | Tumor | Volume | |
|---|---|---|---|---|---|---|---|---|
| Treatment | Dose | Units | Cotreatment | Dose | Units | Volume | Decrease | Fa |
| Control | | | | | | 1418.6 | 0.0 | 0.000 |
| Cisplatin | 1 | mg/kg | | | | 1300.4 | 118.2 | 0.083 |
| Cisplatin | 2 | mg/kg | | | | 1340.1 | 78.5 | 0.055 |
| huCBE11 | 50 | μg | | | | 869.7 | 548.9 | 0.387 |
| huCBE11 | 500 | μg | | | | 614.7 | 803.9 | 0.567 |
| huCBE11 | 50 | μg | Cisplatin | 1 | mg/kg | 490.4 | 928.2 | 0.654 |
| huCBE11 | 50 | μg | Cisplatin | 2 | mg/kg | 354.5 | 1064.1 | 0.750 |
| huCBE11 | 500 | μg | Cisplatin | 1 | mg/kg | 410.9 | 1007.7 | 0.710 |
| huCBE11 | 500 | μg | Cisplatin | 2 | mg/kg | 275.0 | 1143.6 | 0.806 |

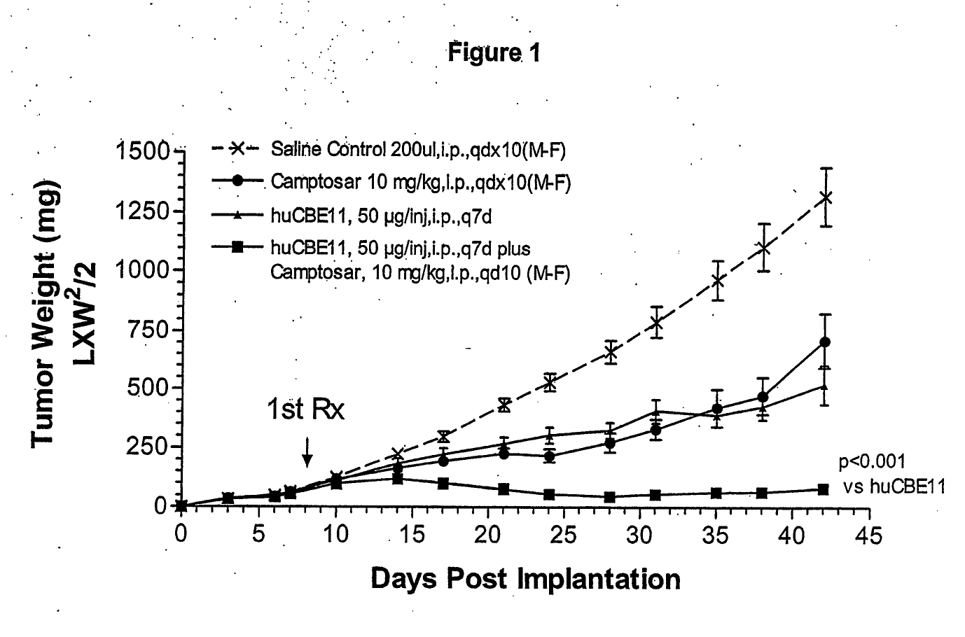

The combination of 500 μg huCBE11 and 2 mg/kg cisplatin or 1 mg/kg cisplatin produced statistically significant (P<0.01 and P<0.05, respectively) lower WiDr tumor weights compared with 500 μg huCBE11 alone. The combination of 50 μg huCBE11 and 2 mg/kg cisplatin or 1 mg/kg cisplatin also produced statistically significant (P<0.001 and P<0.01, respectively) lower WiDr tumor weights compared with 50 μg huCBE11 alone. On day 44, tumor weight was significantly less following treatment with the combination of huCBE11 500 μg plus cisplatin 2 mg/kg (P<0.01) (FIG. 1) or huCBE11 500 μg plus cisplatin 1 mg/kg ) (P<0.05) than with huCBE11 500 μg alone. In addition, mean tumor weights were significantly less following treatment with the combination groups of huCBE11 50 μg plus cisplatin 2 mg/kg (P<0.001) and huCBE11 50 μg plus cisplatin 1 mg/kg (P<0.01) than in the huCBE11 50 μg alone group.

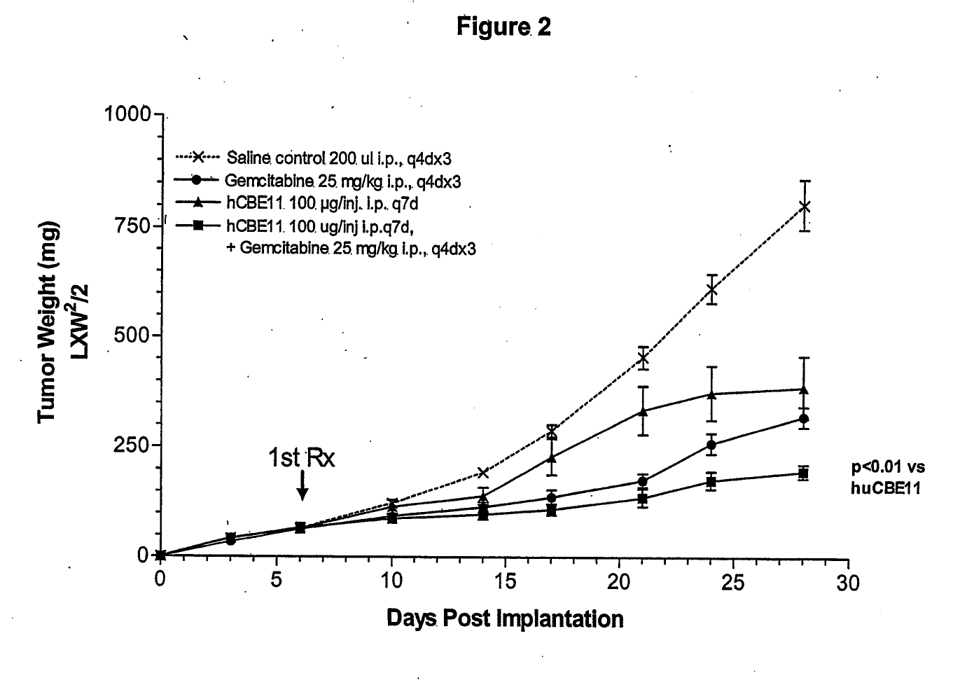

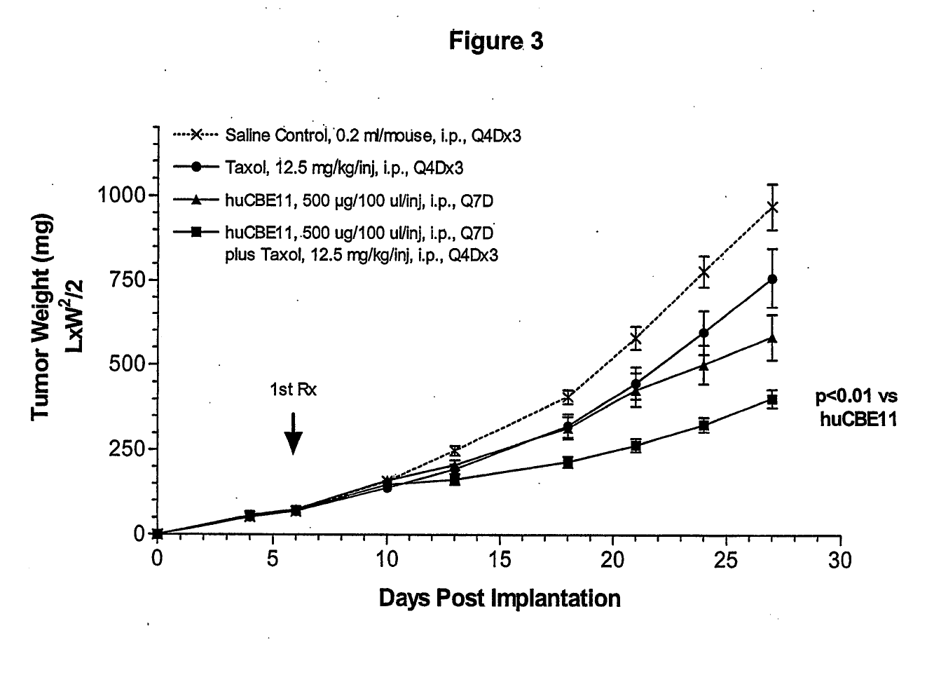

The combination of huCBE11 plus cisplatin at all dose combinations tested was determined to be active in the WiDr model based on the NCI activity criteria (% T/C 42 or less). The % T/C was <42% in from day 24 (38.4%) to day 44 (19.4%) in the huCBE11 500 μg plus cisplatin 2 mg/kg. The % T/C was <42% in from day 30 (37.3%) to day 44 (29.0%) in the huCBE11 500 μg plus cisplatin 1 mg/kg (FIG. 2). The % T/C was <42% in from day 27 (40.5%) to day 44 (25.0%) in the huCBE11 50 μg plus cisplatin 2 mg/kg (FIG. 3). The % T/C was <42% in from day 34 (40.0%) to day 44 (34.6%) in the huCBE11 50 μg plus cisplatin 1 mg/kg.

Using the tumor weight data obtained from these studies, statistical comparison was performed to determine whether the combination of huCBE11 plus cisplatin resulted in potentiation. Individual tumor volumes on day 44 (Table 5) were used to calculate fractional inhibition of tumor volume (i.e., Fa) for each animal (Table 6). Testing for statistically significant potentiation required the calculation of Fa for each animal. Individual tumor volumes (Table 5) were used to calculate fractional inhibition of tumor volume (Fa) for each animal (Table 6). As shown in Table 4, the Fa was calculated as (control group mean tumor volume−individual animal tumor volume)÷control group mean tumor volume. The expected additive Fa for a combination treatment was taken to be the sum of mean Fa's from groups receiving either element of the combination (huCBE11 or cisplatin). The difference between a combination treatment's actual efficacy and that which would be expected if the treatments were merely additive was also calculated (Table 6). A two-tailed one-sample t-test was used to determine whether the combination treatment produced a mean Fa that was statistically significantly different from the expected additive value (Table 6). All combination treatment regimens using huCBE11 plus cisplatin employed in the current study statistically significantly potentiated antitumor efficacy when compared to expected additive antitumor effects.

TABLE 5
Individual tumor volumes at day 44

| | | | | | | huCBE11 | huCBE11 | huCBE11 | huCBE11 |
|---|---|---|---|---|---|---|---|---|---|
| | | | | | | 50 μg + | 50 μg + | 500 μg + | 500 μg + |
| | | Cisplatin | Cisplatin | huCBE11 | huCBE11 | Cisplatin | Cisplatin | Cisplatin | Cisplatin |
| | Control | 1 mg/kg | 2 mg/kg | 50 μg | 500 μg | 1 mg/kg | 2 mg/kg | 1 mg/kg | 2 mg/kg |
| | 1503.6 | 1280.0 | 970.2 | 842.6 | 726.2 | 445.5 | 305.8 | 460.1 | 292.7 |
| | 1123.2 | 1038.3 | 1204.2 | 686.0 | 645.6 | 638.0 | 632.2 | 388.2 | 159.9 |
| | 983.3 | 2096.4 | 1301.9 | 487.5 | 99.1 | 548.5 | 420.8 | 285.4 | 141.6 |
| | 1052.2 | 930.1 | 1505.3 | 802.7 | 944.8 | 359.8 | 286.8 | 314.2 | 271.7 |
| | 1228.2 | 1383.3 | 1469.5 | 624.9 | 534.1 | 398.7 | 407.0 | 359.3 | 690.3 |
| | 1413.9 | 1440.7 | 541.4 | 712.0 | 333.2 | 437.1 | 247.0 | 418.9 | 691.6 |
| | 1649.3 | 1220.3 | 1829.8 | 1216.2 | 469.4 | 830.0 | 463.2 | 388.0 | 175.8 |
| | 703.0 | 1274.3 | 2160.2 | 580.7 | 655.8 | 639.3 | 318.3 | 427.3 | 28.1 |
| | 1626.8 | 1103.9 | 1076.4 | 1866.3 | 1014.0 | 480.0 | 250.4 | 424.8 | 145.4 |
| | 1285.5 | 1236.7 | 1342.0 | 878.7 | 725.3 | 126.8 | 213.5 | 642.5 | 152.6 |
| | 1215.6 | | | | | | | | |
| | 2294.6 | | | | | | | | |
| | 2271.9 | | | | | | | | |
| | 974.4 | | | | | | | | |
| | 2039.3 | | | | | | | | |
| | 1713.9 | | | | | | | | |
| | 741.1 | | | | | | | | |
| | 1467.8 | | | | | | | | |
| | 1757.7 | | | | | | | | |
| | 1327.3 | | | | | | | | |
| Ave.: | 1418.6 | 1300.4 | 1340.1 | 869.7 | 614.7 | 490.4 | 354.5 | 410.9 | 275.0 |

TABLE 6
Individual fractional inhibition of tumor volume

| | | | | | huCBE11 | huCBE11 | huCBE11 | huCBE11 |
|---|---|---|---|---|---|---|---|---|
| | | | | | 50 μg + | 50 μg + | 500 μg + | 500 μg + |
| | Cisplatin | Cisplatin | huCBE11 | huCBE11 | Cisplatin | Cisplatin | Cisplatin | Cisplatin |
| | 1 mg/kg | 2 mg/kg | 50 μg | 500 μg | 1 mg/kg | 2 mg/kg | 1 mg/kg | 2 mg/kg |
| | 0.098 | 0.316 | 0.406 | 0.488 | 0.686 | 0.784 | 0.676 | 0.794 |
| | 0.268 | 0.151 | 0.516 | 0.545 | 0.550 | 0.554 | 0.726 | 0.887 |
| | −0.478 | 0.082 | 0.656 | 0.930 | 0.613 | 0.703 | 0.799 | 0.900 |
| | 0.344 | −0.061 | 0.434 | 0.334 | 0.746 | 0.798 | 0.778 | 0.808 |
| | 0.025 | −0.036 | 0.559 | 0.624 | 0.719 | 0.713 | 0.747 | 0.513 |
| | −0.016 | 0.618 | 0.498 | 0.765 | 0.692 | 0.826 | 0.705 | 0.512 |
| | 0.140 | −0.290 | 0.143 | 0.669 | 0.415 | 0.673 | 0.726 | 0.876 |
| | 0.102 | −0.523 | 0.591 | 0.538 | 0.549 | 0.776 | 0.699 | 0.980 |
| | 0.222 | 0.241 | −0.316 | 0.285 | 0.662 | 0.824 | 0.701 | 0.898 |
| | 0.128 | 0.054 | 0.381 | 0.489 | 0.911 | 0.849 | 0.547 | 0.892 |
| Ave.: | 0.083 | 0.055 | 0.387 | 0.567 | 0.654 | 0.750 | 0.710 | 0.806 |
| Additive: | | | | | 0.470 | 0.442 | 0.650 | 0.622 |
| Difference: | | | | | 0.184 | 0.308 | 0.060 | 0.184 |
| | Two-Tailed One-Sample T-Test | | | | | | | |
| T-Value: | | | | | 4.345 | 10.806 | 2.784 | 3.572 |
| DF: | | | | | 9 | 9 | 9 | 9 |
| P-Value: | | | | | 0.0019 | <0.0001 | 0.0213 | 0.0060 |

All of the combinations employed in the huCBE11/cisplatin study produced statistically significant supra-additive inhibition of tumor volume (Table 6). When combined with a 50 μg or 500 μg dose of huCBE11, cisplatin doses of 1 and 2 mg/kg produced effects that were statistically significantly supra-additive. These combinations significantly potentiated the antitumor effect of huCBE11. In sum, the combination treatment of the LTβ receptor-activating mAb huCBE11 and the chemotherapeutic agent cisplatin in athymic nude mice implanted subcutaneously with WiDr human colorectal adenocarcinoma showed significantly improved results, i.e., showed potentiation, in comparison to huCBE11 and cisplatin administered alone.

### Example 2

### Antitumor Efficacy of LTβR Agonist in Combination with Anthracycline Analog Chemotherapeutic Agent

Antitumor Efficacy of Combination of huCBE11 with Adriamycin

In order to determine whether administration of an anthracycline analog chemotherapeutic agent, e.g., adriamycin, in combination with huCBE11 has supra-additive antitumor activity, e.g., synergistic or potentiating, adriamycin was administered in combination with huCBE11 using the WiDr xenograft tumor model.

A dose ranging study was initially performed to determine the appropriate adriamycin and huCBE11 dose(s) for studying the combined antitumor effects of adriamycin and huCBE11, as well as to determine the individual effects of each drug alone. Increasing doses of adriamycin from 1 mg/kg to 6 mg/kg were administered via intraperitoneal injection to athymic nude mice implanted subcutaneously with WiDr tumor cells (day 0). At all tested doses, adriamycin was determined to be inactive as a chemotherapeutic agent in the WiDr model based on the National Cancer Institute (NCI) activity criteria (percent test/control [% T/C] at or below 42). On day 42 (end of study), there was no significant difference between the adriamycin groups and the vehicle control group. In a separate study, adriamycin also did not produce a significant inhibition of tumor growth at either 6 mg/kg or 4 mg/kg on day 35 (day of evaluation). Thus, adriamycin did not produce a significant inhibition of the WiDr tumor, and there was no significant difference between the adriamycin groups and the saline control group.

In parallel studies, huCBE11 was found to inhibit tumor growth on day 42 at doses of 500 μg, 100 μg, and 50 μg, and was determined to be active as a chemotherapeutic agent in the WiDr model based on the NCI activity criteria. WiDr tumor weight was statistically significantly lower in all of the huCBE11 antibody test groups as compared with the vehicle control group at the end of the study (day 42). The % T/C was observed to be <42% (thus meeting the NCI activity criteria) on days 32 to 42 in the huCBE11 500 μg and 100 μg groups.

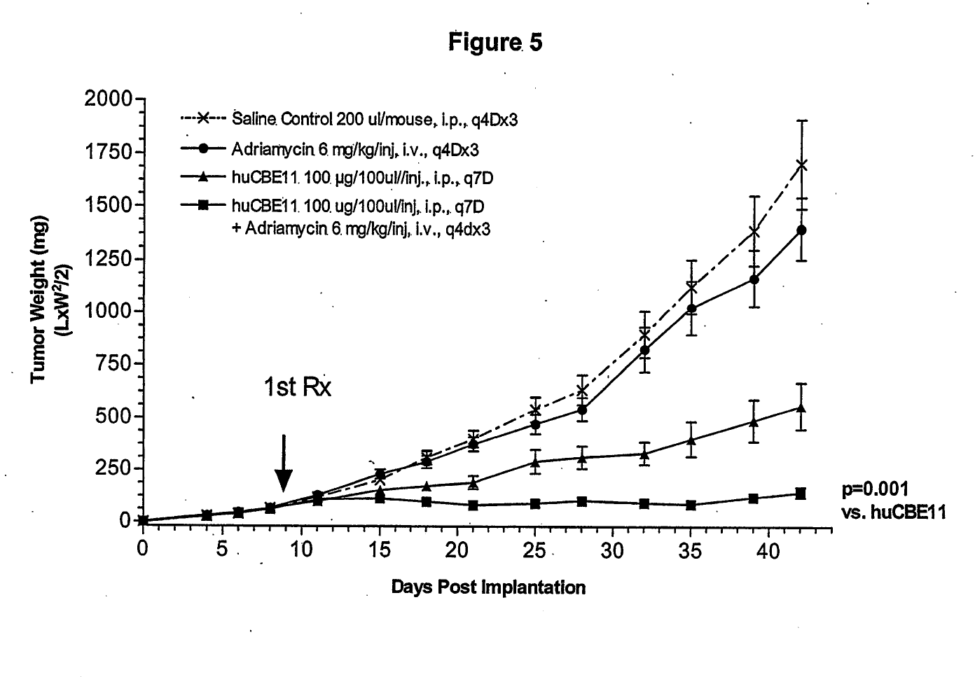

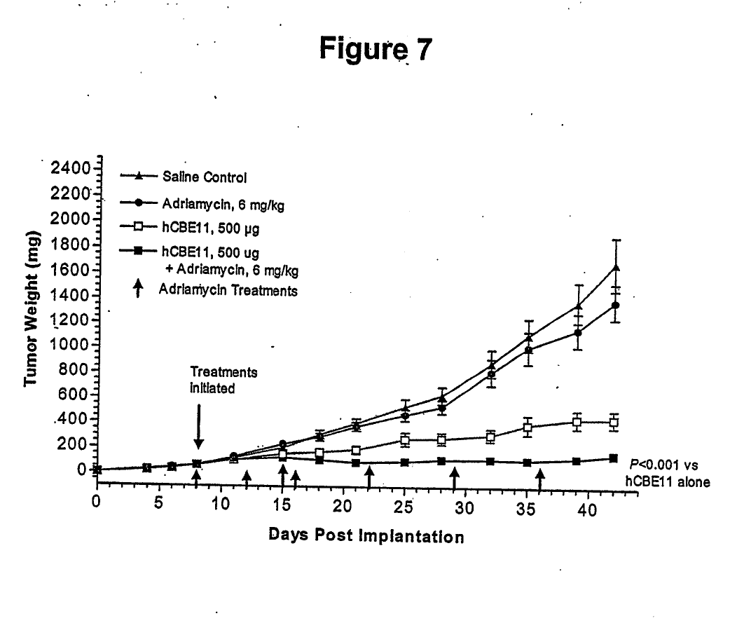

All combinations of huCBE11 plus adriamycin tested were determined to be active in the WiDr model based on the NCI criteria (% T/C at or below 42). The combination groups of huCBE11 100 μg/injection or 500 μg/injection with adriamycin 6 mg/kg (P<0.001) and huCBE11 50 μg/injection with Adriamycin 6 mg/kg or 4 mg/kg (P<0.05) had statistically significantly lower tumor weights than the corresponding huCBE11 alone groups. The % T/C was below 42% from day 18, 21, or 32 to 42 in all combination dose groups, with a low of 8.4% on day 42 in the hCBE11 100 μg plus adriamycin 6 mg/kg group. Tumor weight was observed to be statistically significantly (P<0.001) lower at the end of study, on Day 42, in the huCBE11 500 μg or 100 μg plus adriamycin 6 mg/kg combination groups than in the respective huCBE11 alone groups (FIG. 5 and FIG. 7).

Following activity studies for adriamycin and huCBE11, studies were performed to better characterize the observed effect of administration of the combination therapy. Using the WiDr human colorectal tumor growth model, experimental nude athymic mice xenografted with WiDr human colorectal tumors were administered 50, 100, or 500 μg huCBE11 alone or in combination with 4 or 6 mg/kg of adriamycin. Antitumor efficacy was determined by comparing each treatment group's tumor volume with the control group's tumor volume. Mean tumor volume decrease was calculated as the difference between control group and treatment group mean tumor volume. The fractional inhibition of tumor volume, i.e., the fraction affected (Fa), was calculated by dividing the treatment group mean tumor volume decrease by the control group mean tumor volume. An Fa of 1.000 indicated complete inhibition of the tumor. Table 7 shows the dose-effect relationships for separate and combination treatments.

TABLE 7
Dose-Effect Relationships for huCBE11 and adriamycin combination treatment
groups

| | | | | | | Tumor | Volume | |
|---|---|---|---|---|---|---|---|---|
| Treatment | Dose | Units | Cotreatment | Dose | Units | Volume | Decrease | Fa |
| Control | | | | | | 1124.0 | 0.0 | 0.000 |
| Adriamycin | 4 | mg/kg | | | | 1353.0 | −229.0 | −0.204 |
| Adriamycin | 6 | mg/kg | | | | 1023.5 | 100.5 | 0.089 |
| huCBE11 | 50 | μg | | | | 633.2 | 490.8 | 0.437 |
| huCBE11 | 100 | μg | | | | 399.5 | 724.5 | 0.645 |
| huCBE11 | 500 | μg | | | | 396.0 | 728.0 | 0.648 |
| huCBE11 | 50 | μg | Adriamycin | 4 | mg/kg | 350.3 | 773.7 | 0.688 |
| huCBE11 | 50 | μg | Adriamycin | 6 | mg/kg | 258.9 | 865.1 | 0.770 |
| huCBE11 | 100 | μg | Adriamycin | 4 | mg/kg | 356.5 | 767.5 | 0.683 |
| huCBE11 | 100 | μg | Adriamycin | 6 | mg/kg | 86.5 | 1037.5 | 0.923 |
| huCBE11 | 500 | μg | Adriamycin | 4 | mg/kg | 288.9 | 835.1 | 0.743 |
| huCBE11 | 500 | μg | Adriamycin | 6 | mg/kg | 118.7 | 1005.3 | 0.894 |

Treatment of mice bearing the WiDr tumor with adriamycin alone did not produce dose-responsive antitumor efficacy; therefore, synergism of the huCBE11+adriamycin combination could not be formally assessed by calculating the Combination Index (Chou and Talalay (1984) Adv. Enz. Regul. 22: 27).

Potentiation by combination treatments was assessed by determining whether the huCBE11/adriamycin combination treatment produced efficacy that was statistically significantly supra-additive when compared to the sum of efficacy produced by the individual treatments. Testing for statistically significant potentiation required the calculation of Fa for each animal. Individual tumor volumes (Table 8) were used to calculate fractional inhibition of tumor volume (Fa) for each animal (Table 9) on day 35. As shown above in Table 7, the Fa was calculated as (control group mean tumor volume−individual animal tumor volume)÷control group mean tumor volume. The expected additive Fa for a combination treatment was taken to be the sum of mean Fa's from groups receiving either element of the combination (huCBE11 or adriamycin). The difference between a combination treatment's actual efficacy and that which would be expected if the treatments were merely additive was also calculated (Table 9). A two-tailed one-sample t-test was used to determine whether the combination treatment produced a mean Fa that was statistically significantly different from the expected additive value (Table 9).

TABLE 8
Individual and mean tumor volumes for calculation of fractional inhibition at day 35

| | | | | | | huCBE11 | huCBE11 | huCBE11 | huCBE11 | huCBE11 | huCBE11 |
|---|---|---|---|---|---|---|---|---|---|---|---|
| | | | | | | 50 μg + | 50 μg + | 100 μg + | 100 μg + | 500 μg + | 500 μg + |
| | Adria | Adria | huCBE11 | huCBE11 | huCBE11 | Adria | Adria | Adria | Adria | Adria | Adria |
| Control | 4 mg/kg | 6 mg/kg | 50 μg | 100 μg | 500 μg | 4 mg/kg | 6 mg/kg | 4 mg/kg | 6 mg/kg | 4 mg/kg | 6 mg/kg |
| 975.4 | 842.1 | 537.2 | 289.2 | 990.1 | 778.7 | 205.8 | 70.6 | 65.1 | 57.2 | 432.8 | 81.7 |
| 2233.6 | 548.3 | 1881.7 | 754.4 | 171.4 | 296.8 | 476.1 | 128.7 | 602.9 | 110.3 | 180.7 | 151.9 |
| 909.0 | 1087.2 | 1455.4 | 311.9 | 267.9 | 0.1 | 284.8 | 259.1 | 128.5 | 74.7 | 209.2 | 76.7 |
| 1069.4 | 1582.5 | 1017.1 | 375.4 | 208.9 | 280.9 | 406.8 | 451.8 | 799.1 | 155.9 | 273.4 | 80.0 |
| 879.1 | 2687.5 | 696.6 | 381.9 | 245.8 | 781.0 | 561.2 | 147.0 | 241.3 | 38.9 | 217.4 | 120.1 |
| 610.1 | 528.1 | 605.1 | 409.6 | 293.4 | 272.3 | 281.4 | 135.2 | 426.8 | 38.9 | 248.1 | 117.4 |
| 761.8 | 1865.5 | 901.8 | 766.4 | 281.4 | 537.5 | 191.2 | 281.9 | 601.7 | 131.1 | 277.0 | 164.7 |
| 647.5 | 1277.8 | 889.3 | 1688.6 | 737.5 | 273.7 | 340.8 | 319.0 | 188.2 | 59.8 | 130.9 | 100.2 |
| 2065.7 | 1550.2 | 1059.5 | 762.8 | 457.4 | 368.7 | 369.8 | 112.7 | 202.8 | 126.5 | 758.7 | 117.8 |
| 583.7 | 1560.9 | 1191.2 | 592.2 | 341.5 | 370.6 | 385.1 | 682.7 | 308.3 | 71.3 | 160.7 | 175.9 |
| 1028.3 | | | | | | | | | | | |
| 1024.9 | | | | | | | | | | | |
| 777.8 | | | | | | | | | | | |
| 1795.6 | | | | | | | | | | | |
| 1817.0 | | | | | | | | | | | |
| 1434.7 | | | | | | | | | | | |
| 2087.6 | | | | | | | | | | | |
| 908.0 | | | | | | | | | | | |
| 684.4 | | | | | | | | | | | |
| 187.0 | | | | | | | | | | | |

Mean

| | | | | | | | | | | | |
|---|---|---|---|---|---|---|---|---|---|---|---|
| 1124.0 | 1353.0 | 1023.5 | 633.2 | 399.5 | 396.0 | 350.3 | 258.9 | 356.5 | 86.5 | 288.9 | 118.7 |

TABLE 9
Fractional Inhibition of Tumor Volume

| | | | | | | huCBE11 | huCBE11 | huCBE11 | huCBE11 | huCBE11 | |
|---|---|---|---|---|---|---|---|---|---|---|---|
| | | | | | | 50 μg + | 50 μg + | 100 μg + | 100 μg + | 500 μg + | huCBE11 500 μg + |
| | Adria | Adria | huCBE11 | huCBE11 | huCBE11 | Adria | Adria | Adria | Adria | Adria | Adria |
| | 4 mg/kg | 6 mg/kg | 50 μg | 100 μg | 500 μg | 4 mg/kg | 6 mg/kg | 4 mg/kg | 6 mg/kg | 4 mg/kg | 6 mg/kg |
| | 0.251 | 0.522 | 0.743 | 0.119 | 0.307 | 0.817 | 0.937 | 0.942 | 0.949 | 0.615 | 0.927 |
| | 0.512 | −0.674 | 0.329 | 0.848 | 0.736 | 0.576 | 0.885 | 0.464 | 0.902 | 0.839 | 0.865 |
| | 0.033 | −0.295 | 0.723 | 0.762 | 1.000 | 0.747 | 0.769 | 0.886 | 0.934 | 0.814 | 0.932 |
| | −0.408 | 0.095 | 0.666 | 0.814 | 0.750 | 0.638 | 0.598 | 0.289 | 0.861 | 0.757 | 0.929 |
| | −1.391 | 0.380 | 0.660 | 0.781 | 0.305 | 0.501 | 0.869 | 0.785 | 0.965 | 0.807 | 0.893 |
| | 0.530 | 0.462 | 0.636 | 0.739 | 0.758 | 0.750 | 0.880 | 0.620 | 0.965 | 0.779 | 0.896 |
| | −0.660 | 0.198 | 0.318 | 0.750 | 0.522 | 0.830 | 0.749 | 0.465 | 0.883 | 0.754 | 0.853 |
| | −0.137 | 0.209 | −0.502 | 0.344 | 0.756 | 0.697 | 0.716 | 0.833 | 0.947 | 0.884 | 0.911 |
| | −0.379 | 0.057 | 0.321 | 0.593 | 0.672 | 0.671 | 0.900 | 0.820 | 0.887 | 0.325 | 0.895 |
| | −0.389 | −0.060 | 0.473 | 0.696 | 0.670 | 0.657 | 0.393 | 0.726 | 0.937 | 0.857 | 0.844 |
| Average: | −0.204 | 0.089 | 0.437 | 0.645 | 0.648 | 0.688 | 0.770 | 0.683 | 0.923 | 0.743 | 0.894 |
| Additive: | — | — | — | — | — | 0.437 | 0.526 | 0.645 | 0.734 | 0.648 | 0.737 |
| Difference: | — | — | — | — | — | 0.252 | 0.244 | 0.038 | 0.189 | 0.095 | 0.157 |

2-Tailed 1-Sample T-Test: Significantly Different from Additive?

| | | | | | | | | | | | |
|---|---|---|---|---|---|---|---|---|---|---|---|
| T-Value: | — | — | — | — | — | 7.719 | 4.577 | 0.559 | 16.182 | 1.827 | 15.687 |
| DF: | — | — | — | — | — | 9 | 9 | 9 | 9 | 9 | 9 |
| P-Value: | — | — | — | — | — | <0.0001 | 0.0013 | 0.5901 | <0.0001 | 0.1010 | <0.0001 |

As shown in Table 9, use of the two-tailed, one sample t-test demonstrated supra-additive effects for a number of huCBE11:adriamycin combination treatments over those of the huCBE11 antibody alone. Both 4 and 6 mg/kg adriamycin doses produced supra-additive effects when combined with 50 μg doses of huCBE11 (P<0.0001 and P=0.0013, respectively). Adriamycin 6 mg/kg produced supra-additive effects when combined with either 100 μg or 500 μg doses of huCBE11 (P<0.0001). These combinations were therefore observed to significantly potentiate the antitumor effect of huCBE11.

A majority of combination treatment regimens using huCBE11 plus adriamycin employed in the current study statistically significantly potentiated antitumor efficacy when compared to expected additive antitumor effects. The combination treatment of the LTβ receptor-activating mAb huCBE11 and the chemotherapeutic agent adriamycin in athymic nude mice implanted subcutaneously with WiDr human colorectal adenocarcinoma showed significantly improved results in comparison to huCBE11 and adriamycin administered alone. These combinations significantly potentiated the antitumor effect of huCBE11.

### Example 3

### Antitumor Efficacy of LTβR Agonist in Combination With Topoisomerase I Chemotherapeutic Agent

A. Antitumor Efficacy of Combination huCBE11/Camptosar Therapy in WiDr Xenograft Model

In order to determine whether administration of a topoisomerase I chemotherapeutic agent, e.g., Camptosar (also referred to as irinotecan), in combination with huCBE11 has supra-additive antitumor activity, e.g., synergistic or potentiating activity, Camptosar was administered in combination with huCBE11 using the WiDr murine model to test as a cancer therapeutic.

A dose ranging study was performed to determine the appropriate Camptosar and huCBE11 dose(s) and to determine the individual activity of each drug alone. Increasing doses of Camptosar from 1.8 mg/kg to 10 mg/kg were administered to athymic nude mice implanted subcutaneously with WiDr tumor cells (day 0). Camptosar produced a statistically significant inhibition of WiDr tumor growth on Day 43, at 10 mg/kg (P<0.001), 6 mg/kg (P<0.01), and 3 mg/kg (P<0.05) compared with the vehicle control. The % T/C was >45% at all other evaluations in all dose groups, except on Days 24 to 31 in the Camptosar 10 mg/kg group when it fell to 41%. The results from the dosing study determined that Camptosar was inactive in the WiDr model by the activity criteria of the National Cancer Institute (NCI; percent test/control [% T/C] of 42 or less constitutes activity).

In parallel studies, huCBE11 was found to inhibit tumor growth on day 43 at doses of 500 μg (P<0.001), 50 μg (P<0.001), and 5 μg (P<0.05). The % T/C was <42% on days 35 or 38 to 42 in the huCBE11 500 μg and 50 μg groups. Compared with the vehicle control, huCBE11 produced a statistically significant inhibition of tumor growth at doses of 500 μg (P<0.001), 50 μg (P<0.001), and 5 μg (P<0.05), as well as at 10 mg/kg (P<0.01) and 6 mg/kg (P<0.05) at the end of study and 3 mg/kg (P<0.05). The % T/C was <42% on Days 35/38 to 42 in the hCBE11 500 μg and 50 μg groups. The results demonstrated that huCBE11 was active in the WiDr model based on the NCI activity criteria (% T/C of 42 or less).

Analyses of the combined activity of huCBE11 and Camptosar were also performed. On Day 42, tumor weight was significantly less following treatment with the combination of huCBE11 50 μg and Camptosar 10 mg/kg than with huCBE11 50 μg alone (P<0.001) (see FIG. 1). In addition, mean tumor weights in the combination groups of huCBE11 50 μg plus Camptosar 6 mg/kg (P<0.01) and huCBE11 50 μg plus Camptosar 3 mg/kg (P<0.05) differed significantly from mean tumor weight in the huCBE11 50 μg alone group. The % T/C fell below 42% by Day 17 and was 6.2% on Day 42 in the huCBE11 50 μg plus Camptosar 10 mg/kg group. In addition, the % T/C was <42% on Days 17 to 42 in the hCBE11 50 μg plus Camptosar 6 mg/kg group and on Days 24 to 42 in the huCBE11 50 μg plus Camptosar 3 mg/kg groups.

There were no statistically significant differences in mean tumor weight between the combined therapy groups of huCBE11 18 μg plus Camptosar 10 mg/kg, huCBE11 10.5 μg plus Camptosar 6 mg/kg, or huCBE11 5.4 μg plus Camptosar 3 mg/kg and the respective Camptosar dose alone on Day 42. The % T/C was <42% only in the huCBE11 18 μg plus Camptosar 10 mg/kg group (Days 21-42).

In sum, it was determined that the combination of huCBE11 and Camptosar was active in the WiDr model based on the NCI activity criteria (% T/C of 42 or less). Weight of pre-established WiDr human colorectal tumors was statistically significantly less following treatment with the following combinations of huCBE11 and the chemotherapeutic agent Camptosar than with huCBE11 alone:

(1) 50 μg huCBE11 plus 10 mg/kg Camptosar compared with 50 μg hCBE11 alone (P<0.001), and % T.C was <42% on Days 17 to 42 (low of 6.2%);

(2) 50 μg hCBE11 plus 6 mg/kg Camptosar compared with 50 μg hCBE11 alone (P<0.01), and % T.C was <42% on Days 17 to 42; and

(3) 50 μg hCBE11 plus 3 mg/kg Camptosar compared with 50 μg hCBE11 alone (P<0.05), and % T/C was <42% on Days 24 to 42.

Supra-additive studies were performed to determine whether huCBE11 and Camptosar could act synergistically using nude athymic mice implanted with WiDr human colorectal tumor growth model. Doses of 10 mg/kg, 6 mg/kg, or 3 mg/kg of Camptosar were chosen for these huCBE11/Camptosar combination studies. All tumor data used to calculate Fa values were taken at day 21. Antitumor efficacy was determined by comparing each treatment group's tumor volume with the control group's tumor volume. Mean tumor volume decrease was calculated as the difference between control group and treatment group mean tumor volume. The fractional inhibition of tumor volume, Fraction affected (Fa), was calculated by dividing treatment group mean tumor volume decrease by control group mean tumor volume. An Fa of 1.000 would indicate complete inhibition of the tumor. Table 10 shows the dose-effect relationships for separate and combination treatments. The Fa values obtained were then used for assessment of both synergy and potentiation for combination huCBE11 and Camptosar treatment.

TABLE 10
Dose-Effect Relationships of huCBE11 and Camptosar combination treatment

| | | | | | | | Volume | |
|---|---|---|---|---|---|---|---|---|
| Treatment | Dose | Units | Cotreat | Dose | Units | Tumor Volume | Decrease | Fa |
| Control | | | | | | 434.0 | 0.0 | 0.000 |
| Camptosar | 3 | mg/kg | | | | 330.9 | 103.1 | 0.238 |
| Camptosar | 6 | mg/kg | | | | 242.8 | 191.2 | 0.441 |
| Camptosar | 10 | mg/kg | | | | 227.2 | 206.8 | 0.476 |
| huCBE11 | 5 | μg | | | | 319.0 | 115.0 | 0.265 |
| huCBE11 | 50 | μg | | | | 267.8 | 166.2 | 0.383 |
| huCBE11 | 500 | μg | | | | 252.5 | 181.5 | 0.418 |
| huCBE11 | 5.4 | μg | Camptosar | 3 | mg/kg | 283.1 | 150.9 | 0.348 |
| huCBE11 | 10.5 | μg | Camptosar | 6 | mg/kg | 237.6 | 196.4 | 0.453 |
| huCBE11 | 18 | μg | Camptosar | 10 | mg/kg | 135.5 | 298.5 | 0.688 |
| huCBE11 | 50 | μg | Camptosar | 3 | mg/kg | 188.0 | 246.0 | 0.567 |
| huCBE11 | 50 | μg | Camptosar | 6 | mg/kg | 103.2 | 330.8 | 0.762 |
| huCBE11 | 50 | μg | Camptosar | 10 | mg/kg | 78.0 | 356.0 | 0.820 |

Those doses used to assess drug potentiation in the current study were not restricted to fixed-ratio combination. Testing for statistically significant potentiation required the calculation of Fa for each animal. Individual tumor volumes (Table 41) were used to calculate fractional inhibition of tumor volume (Fa) for each animal (Table 42). Fa was calculated as (control group mean tumor volume−individual animal tumor volume)÷control group mean tumor volume. The expected additive Fa for a combination treatment was taken to be the sum of mean Fa's from groups receiving either element of the combination. The difference between a combination treatment's actual efficacy and that which would be expected if the treatments were merely additive was also calculated (Table 42). A two-tailed one-sample t-test was used to determine whether the combination treatment produced a mean Fa that was statistically significantly different from the expected additive value (Table 42).

TABLE 41
Individual Tumor Volumes

| | Control | Camp 3 | Camp 6 | Camp 10 | hCBE 50 | hCBE 50 + Camp 3 | hCBE 50 + Camp 6 | hCBE 50 + Camp 10 |
|---|---|---|---|---|---|---|---|---|
| | 607.4 | 463.6 | 177.9 | 363.3 | 171.3 | 137.9 | 175.0 | 102.2 |
| | 588.4 | 265.8 | 197.8 | 209.9 | 356.5 | 150.7 | 136.3 | 123.1 |
| | 356.7 | 211.5 | 386.4 | 137.2 | 440.3 | 112.9 | 93.0 | 118.8 |
| | 239.1 | 343.5 | 167.6 | 252.0 | 254.0 | 124.4 | 81.7 | 34.3 |
| | 353.0 | 393.6 | 313.8 | 152.8 | 326.6 | 261.7 | 16.7 | 80.3 |
| | 352.7 | 288.0 | 246.9 | 232.5 | 170.5 | 288.5 | 70.8 | 31.8 |
| | 638.2 | 297.6 | 303.1 | 283.6 | 244.3 | 230.1 | 73.3 | 49.2 |
| | 573.4 | 400.1 | 228.8 | 184.4 | 231.8 | 197.0 | 77.0 | 37.4 |
| | 508.8 | 317.4 | 207.9 | 271.4 | 178.1 | 163.6 | 81.5 | 65.2 |
| | 365.5 | 327.6 | 197.8 | 185.4 | 304.5 | 213.3 | 226.8 | 137.6 |
| | 625.5 | | | | | | | |
| | 356.5 | | | | | | | |
| | 394.0 | | | | | | | |
| | 387.8 | | | | | | | |
| | 576.4 | | | | | | | |
| | 350.6 | | | | | | | |
| | 346.0 | | | | | | | |
| | 283.3 | | | | | | | |
| | 351.9 | | | | | | | |
| | 424.8 | | | | | | | |
| Ave.: | 434.0 | 330.9 | 242.8 | 227.2 | 267.8 | 188.0 | 103.2 | 78.0 |

TABLE 42
Individual Fractional Inhibition of Tumor Volume

| | Camp 3 | Camp 6 | Camp 10 | hCBE 50 | hCBE 50 + Camp 3 | hCBE 50 + Camp 6 | hCBE 50 + Camp 10 |
|---|---|---|---|---|---|---|---|
| | −0.068 | 0.590 | 0.163 | 0.605 | 0.682 | 0.597 | 0.764 |
| | 0.388 | 0.544 | 0.516 | 0.179 | 0.653 | 0.686 | 0.716 |
| | 0.513 | 0.110 | 0.684 | −0.015 | 0.740 | 0.786 | 0.726 |
| | 0.208 | 0.614 | 0.419 | 0.415 | 0.713 | 0.812 | 0.921 |
| | 0.093 | 0.277 | 0.648 | 0.247 | 0.397 | 0.962 | 0.815 |
| | 0.336 | 0.431 | 0.464 | 0.607 | 0.335 | 0.837 | 0.927 |
| | 0.314 | 0.302 | 0.347 | 0.437 | 0.470 | 0.831 | 0.887 |
| | 0.078 | 0.473 | 0.575 | 0.466 | 0.546 | 0.823 | 0.914 |
| | 0.269 | 0.521 | 0.375 | 0.590 | 0.623 | 0.812 | 0.850 |
| | 0.245 | 0.544 | 0.573 | 0.298 | 0.508 | 0.477 | 0.683 |
| Ave.: | 0.238 | 0.441 | 0.476 | 0.383 | 0.567 | 0.762 | 0.820 |
| Additive: | | | | | 0.621 | 0.824 | 0.859 |
| Difference: | | | | | −0.054 | −0.061 | −0.039 |
| | Two-Tailed One-Sample T-Test | | | | | | |
| T-Value: | | | | | −1.246 | −1.404 | −1.319 |
| DF: | | | | | 9 | 9 | 9 |
| P-Value: | | | | | 0.2444 | 0.1940 | 0.2197 |

Treatment of mice bearing the WiDr tumor with Camptosar alone produced dose-responsive antitumor efficacy, therefore synergism of the huCBE11+Camptosar combination could be formally assessed by calculating the Combination Index (CI). Those doses used to assess synergistic drug action were given in a fixed ratio of 0.555:1 mg/kg Camptosar:μg huCBE11. This ratio was based on the ratio of the median effect doses for the two agents determined in the above-mentioned studies. Formal assessment of synergism employed calculation of the Combination Index (CI) using CalcuSyn V1.1 (Biosoft, Cambridge, UK) software for Windows-based dose-effect analysis. As described above, for treatments given in combination, a CI=1 indicates additive efficacy. CI<1 indicates synergism. CI>1 indicates antagonism. Dose-effect relationships (Fa values) used in CI calculations are presented in Table 11.

TABLE 11
Dose-Effect Relationships for Synergism Calculations for Camptosar-huCBE11
in the WiDr Xenograft Model

| Given Separately | | | | Combined (0.555:1) | | |
|---|---|---|---|---|---|---|
| Camptosar | | | | Camptosar | huCBE11 | |
| Dose | Fraction | huCBE11 Dose | Fraction | Dose | Dose | Fraction |
| (mg/kg) | Affected | (μg) | Affected | (mg/kg) | (μg) | Affected |
| 3 | 0.238 | 5 | 0.265 | 3 | 5.4 | 0.348 |
| 6 | 0.441 | 50 | 0.383 | 6 | 10.8 | 0.453 |
| 10 | 0.476 | 500 | 0.418 | 10 | 18.0 | 0.688 |

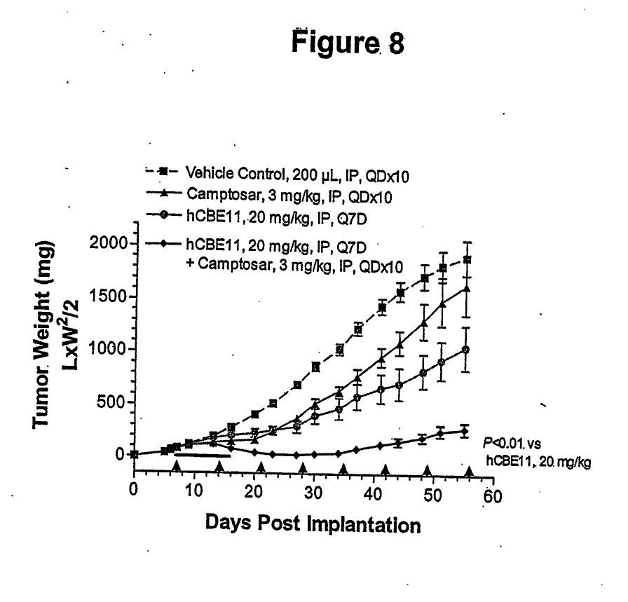

Based on the CI values calculated for Camptosar and huCBE11, it was determined that fixed-ratio combination treatment (0.555 mg/kg: 1 μg) of the Camptosar/huCBE11 combination showed synergistic antitumor efficacy. Potency and shape of the dose-response relation for separate and combination treatments are shown in Tables 12 and 13, respectively. The CI values calculated for the exact level of the experimental doses used in the current study are shown in Table 14. Because the current study employs drugs that are thought to have entirely independent modes of action, mutually nonexclusive CI values were applied. Combination doses using 3 mg/kg Camptosar+5.4 μg huCBE11, 6 mg/kg Camptosar+10.5 μg huCBE11, and 10 mg/kg Camptosar+18 μg huCBE11 showed a synergistic effect. Simulations of the CI over a range of dose levels for the combination are given in Table 15. Combination doses ranging from 3.3 mg/kg Camptosar+6 μg huCBE11 (giving 35% inhibition of tumor volume) to 313 mg/kg Camptosar+563 μg huCBE11 (giving an 99% inhibition of tumor volume) showed a synergistic effect. Overall interpretation of the degree of synergism or antagonism indicated by the CI is given in Table 3. CI as a function of fraction affected is shown in FIG. 8. In sum, fixed-ratio combination treatment (0.555:1) using Camptosar plus huCBE11 showed synergistic antitumor efficacy against established xenografts of the WiDr human colorectal adenocarcinoma when evaluated on Day 21.

TABLE 12
Median effect doses

Median Effect
Dose (95% C.L.)

| | Dose | | Combined |
|---|---|---|---|
| Agent | Units | Given Separately | (0.555:1) |
| Camptosar | mg/kg | 9.8 | 5.7 |
| | | (6.1-15.6) | (4.2-7.7) |
| huCBE11 | μg | 2933 | 10.2 |
| | | (157-54760) | (7.6-13.9) |

TABLE 13
Dose-effect curve characteristics

| | | | |
|---|---|---|---|
| | | Y- | |
| Value | Slope | Intercept | R |

Camptosar

| | | | |
|---|---|---|---|
| Mean | 0.913 | −0.903 | 0.9510 |
| SEM | 0.297 | 0.232 | |

huCBE11

| | | | |
|---|---|---|---|
| Mean | 0.150 | −0.519 | 0.9488 |
| SEM | 0.050 | 0.094 | |

Camptosar + huCBE11

| | | | |
|---|---|---|---|
| Mean | 1.147 | −0.866 | 0.9543 |
| SEM | 0.359 | 0.281 | |

TABLE 14
Calculated Combination Indices (CIs) for Experimental Values

| | | | Mechanisms of Action | | | |
|---|---|---|---|---|---|---|
| Camptosar | huCBE11 | | Mutually | | | |
| Dose | Dose | Fraction | Exclusive | | Nonexclusive | |
| (mg/kg) | (ug) | Affected | CI | Synergism | CI | Synergism |
| 3 | 5.4 | 0.348 | 0.734 | ++ | 0.809 | +++ |
| 6 | 10.8 | 0.453 | 0.769 | +++ | 0.779 | +++ |
| 10 | 18.0 | 0.688 | 0.431 | +++ | 0.431 | +++ |

++ moderate synergism
+++ synergism

TABLE 15
Combination Index (CI) Simulations

| | | | | |
|---|---|---|---|---|
| | | Camptosar | huCBE11 | |
| Fa | CI | (mg/kg) | (μg) | Symbol |

Mutually Exclusive Mechanisms of Action

| | | | | |
|---|---|---|---|---|
| 0.02 | 2.31E+07 | 0.191 | 0.344 | − − − − − |
| 0.05 | 9.40E+04 | 0.44 | 0.79 | − − − − − |
| 0.10 | 1224.405 | 0.8 | 1.5 | − − − − − |
| 0.15 | 84.071 | 1.3 | 2.3 | − − − − − |
| 0.20 | 11.792 | 1.7 | 3.1 | − − − − − |
| 0.25 | 2.812 | 2.2 | 3.9 | − − − |
| 0.30 | 1.185 | 2.7 | 4.9 | − |
| 0.35 | 0.797 | 3.3 | 6.0 | ++ |
| 0.40 | 0.675 | 4.0 | 7.2 | +++ |
| 0.45 | 0.621 | 4.8 | 8.6 | +++ |
| 0.50 | 0.587 | 5.7 | 10.2 | +++ |
| 0.55 | 0.559 | 6.8 | 12.2 | +++ |
| 0.60 | 0.533 | 8.1 | 14.6 | +++ |
| 0.65 | 0.508 | 9.8 | 17.6 | +++ |
| 0.70 | 0.483 | 11.9 | 21.4 | +++ |
| 0.75 | 0.456 | 14.8 | 26.7 | +++ |
| 0.80 | 0.428 | 19.1 | 34.3 | +++ |
| 0.85 | 0.396 | 25.8 | 46.5 | +++ |
| 0.90 | 0.357 | 38.6 | 69.6 | +++ |
| 0.95 | 0.302 | 74.1 | 133.4 | +++ |
| 0.99 | 0.209 | 312.6 | 562.8 | ++++ |

Nonexclusive (Totally Independent Modes of Action)

| | | | | |
|---|---|---|---|---|
| 0.02 | 5.53E+07 | 1.91E−01 | 0.3 | − − − − − |
| 0.05 | 2.00E+05 | 0.437 | 0.8 | − − − − − |
| 0.10 | 2390.925 | 0.837 | 1.5 | − − − − − |
| 0.15 | 155.609 | 1.254 | 2.3 | − − − − − |
| 0.20 | 20.537 | 1.698 | 3.1 | − − − − − |
| 0.25 | 4.354 | 2.183 | 3.9 | − − − − |
| 0.30 | 1.523 | 2.717 | 4.9 | − − − |
| 0.35 | 0.883 | 3.316 | 6.0 | + |
| 0.40 | 0.699 | 3.994 | 7.2 | +++ |
| 0.45 | 0.628 | 4.775 | 8.6 | +++ |
| 0.50 | 0.589 | 5.688 | 10.2 | +++ |
| 0.55 | 0.559 | 6.776 | 12.2 | +++ |
| 0.60 | 0.533 | 8.101 | 14.6 | +++ |
| 0.65 | 0.508 | 9.759 | 17.6 | +++ |
| 0.70 | 0.483 | 11.908 | 21.4 | +++ |
| 0.75 | 0.456 | 14.825 | 26.7 | +++ |
| 0.80 | 0.428 | 19.052 | 34.3 | +++ |
| 0.85 | 0.396 | 25.813 | 46.5 | +++ |
| 0.90 | 0.357 | 38.639 | 69.6 | +++ |
| 0.95 | 0.302 | 74.127 | 133.4 | +++ |
| 0.99 | 0.209 | 312.642 | 562.8 | ++++ |

In sum, the combination treatment of the LTβ receptor-activating mAb huCBE11 and the chemotherapeutic agent Camptosar in athymic nude mice implanted subcutaneously with WiDr human colorectal adenocarcinoma showed significantly improved results in comparison to huCBE11 and Camptosar administered alone. Strikingly, the effect of fixed ratio (0.555 mg/kg Camptosar:1 μg huCBE11) combination treatment with huCBE11 and Camptosar was determined to be synergistic by combination index analysis.

B. Antitumor Efficacy of Combination huCBE11/Camptosar Therapy in KM-20L2 Xenograft Model

An additional human colorectal adenocarcinoma mouse model system, the KM-20L2 model, was also utilized to determine whether administration of a topoisomerase I chemotherapeutic agent, e.g., Camptosar, in combination with huCBE11 has supra-additive antitumor activity, e.g., potentiated or synergistic, when Camptosar was administered in combination with huCBE11.

A dose ranging study was performed to determine the appropriate Camptosar dose(s) and huCBE11 for studying the combined antitumor effects of Camptosar and huCBE11. Increasing doses of Camptosar from 1.8 mg/kg to 10 mg/kg were administered to athymic nude mice implanted subcutaneously with KM-20L2 tumor cells (day 0). Camptosar treatment produced a statistically significant inhibition of KM-20L2 tumor growth at 10 mg/kg (P<0.001) and 6 mg/kg (P<0.01) and 3 mg/kg (P<0.05) on day 33 (end of study). Inhibition was first observed on days 11 to 14. The % T/C was at or below 42% from days 18 through 33 in the 10 mg/kg group, thus meeting the NCI activity criteria. In a separate study, no significant tumor inhibition was observed at the end of day 55 in the Camptosar groups, yet statistically significant inhibition of tumor growth at 6 mg/kg on days 13 through 51 (P<0.001 dayse 13 to 44; P<0.01 day 48; P<0.05 day 51), 3 mg/kg on days 13 through 48 (P<0.001 days 13 to 41; P<0.01 day 44; P<0.05 day 48), and 1.8 mg/kg on days 9 through 44 (P<0.001 days 16 to 27, 41; P<0.01 days 13, 30-37; P<0.05 days 9, 44) was observed, as compared with the saline control group. The % T/C was at or below 42% on Days 16 through 30 in the 6 mg/kg group and on Day 20 in the 3 mg/kg group. Thus, Camptosar was determined to be active in the KM-20L2 tumor model based on the National Cancer Institute (NCI) activity criteria (% T/C at or below 42).

In a parallel dosing study to assess the anti-tumor activity of huCBE11 in the KM-20L2 xenograft model, tumor growth was observed to be significantly (P<0.05) decreased in the huCBE11 2 mg/kg (Days 28 and 33) and 4 mg/kg (Days 21-28) dose groups, as compared with the vehicle control group. In a parallel separate study (in which efficacy of combination therapies were also examined), huCBE11 produced a significant inhibition of tumor growth at the following doses:
(1) 20 mg/kg on Days 16 through 55 (P<0.001 Days 20-48; P<0.01 Days 16, 51, 55),
(2) 2 mg/kg on Days 16 through 55 (P<0.001 Days 16-51; P<0.01 Day 55), and
(3) 0.2 mg/kg on Days 20 through 55 (P<0.001 Days 27-30, 41; P<0.01 Days 20-23, 34-37, 44-48; P<0.05 Days 51-55). The % T/C in the huCBE11 dose groups was >42% throughout the course of both studies. The lowest % T/C observed in groups of the second study was 42.4% in the 20 mg/kg group on Day 27. In sum, huCBE11 was determined to be inactive in the KM-20L2 tumor model based on the NCI activity criteria (% T/C at or below 42). At the end of the dosing study on Day 33, huCBE11 produced a statistically significant inhibition of tumor growth at 2 mg/kg compared with the vehicle control. Inhibition was also observed on Days 21 to 28 in the 4 mg/kg group and on Day 28 in the 2 mg/kg group. In a separate study, significant inhibition of tumor growth was observed at huCBE11 20 mg/kg on Days 16 through 55, 2 mg/kg on Days 16 through 55, and 0.2 mg/kg on Days 20 through 55 compared with the vehicle control. The % T/C fell to a low of 42.4% on Day 27 in the hCBE11 20 mg/kg group.

The combination effect of huCBE11 and Camptosar was also examined. The combination of huCBE11 20 mg/kg and Camptosar 3 mg/kg resulted in a statistically significant decrease in tumor growth compared with huCBE11 20 mg/kg alone (P<0.001 Days 16 to 41, 48; P<0.01 Days 13, 44-55) (FIG. 8). When combined with huCBE11 2 mg/kg, a Camptosar dose of 3 mg/kg (P<0.001, Days 16-44; P<0.01 Days 48-55; P<0.05 Day 13) or 1.8 mg/kg (P<0.001 Day 20; P<0.01 Days 23; P<0.05, Day 16, 27-37) also resulted in a significantly lower mean tumor weight compared with huCBE11 2 mg/kg alone. When combined with huCBE11 0.2 mg/kg, Camptosar 1.8 mg/kg produced a statistically significant (P<0.05) inhibition of tumor growth on Days 13 to 23 compared with huCBE11 0.2 mg/kg alone. In addition, combinations of huCBE11 9.48 mg/kg plus Camptosar 6 mg/kg (P<0.001), huCBE11 4.74 mg/kg plus Camptosar 3 mg/kg (P<0.001), and huCBE11 2.84 mg/kg plus Camptosar 1.8 mg/kg (P<0.01) produced statistically significant tumor growth inhibition at the end of study. The majority of these huCBE11 plus Camptosar combination groups had % T/C of less than 42% from Day 16 or 20 through the end of study. Thus, the combination of huCBE11 and Camptosar was determined to be active in the KM-20L2 tumor model based on the NCI activity criteria (% T/C at or below 42).

To examine whether huCBE11 and Camptosar could either potentiate one another or act synergistically, supra-additive studies were performed using nude athymic mice implanted with KM-20L2 human colon adenocarcinomas in a tumor growth model as described above. Doses of 6 mg/kg, 3 mg/kg, or 1.8 mg/kg of Camptosar were chosen for these huCBE11/Camptosar combination studies. Tumor data used to calculate Fa values were taken at days 9, 13, 16, 20, 23, 27, 30, 34, 37, and 41. Antitumor efficacy was determined by comparing each treatment group's tumor volume with the control group's tumor volume. Mean tumor volume decrease was calculated as the difference between control group and treatment group mean tumor volume. The fractional inhibition of tumor volume, Fraction affected (Fa), was calculated by dividing treatment group mean tumor volume decrease by control group mean tumor volume. An Fa of 1.000 would indicate complete inhibition of the tumor. Table 15 shows the dose-effect relationships for separate and combination treatments across the time course of the experiment. The Fa values obtained were then used in assessment of both synergy and potentiation for combination huCBE11 and Camptosar treatment.

TABLE 15
huCBE11 and Camptosar: Expected Additive Versus Actual Tumor Volume Inhibition

| | Dose | | | | | | | | | | | | |
|---|---|---|---|---|---|---|---|---|---|---|---|---|---|
| | (mg/ | Cotreat- | Dose | Day | | | | | | | | | |
| Treatment | kg) | ment | (mg/kg) | 9 | 13 | 16 | 20 | 23 | 27 | 30 | 34 | 37 | 41 |

Inhibition of Tumor Volume: Mean Fraction Affected (Fa)

| Camptosar | 1.8 | | | 0.144 | 0.196 | 0.278 | 0.417 | 0.357 | 0.340 | 0.260 | 0.250 | 0.254 | 0.212 |
|---|---|---|---|---|---|---|---|---|---|---|---|---|---|
| huCBE11 | 0.2 | | | −0.026 | 0.003 | 0.125 | 0.249 | 0.294 | 0.377 | 0.369 | 0.345 | 0.372 | 0.381 |
| huCBE11 | 0.2 | Camptosar | 1.8 | 0.031 | 0.207 | 0.389 | 0.579 | 0.542 | 0.539 | 0.500 | 0.475 | 0.414 | 0.382 |
| Additive: | | | | 0.118 | 0.199 | 0.403 | 0.666 | 0.651 | 0.718 | 0.629 | 0.595 | 0.626 | 0.592 |
| Difference: | | | | −0.087 | 0.009 | −0.013 | −0.087 | −0.109 | −0.178 | −0.130 | −0.119 | −0.212 | −0.210 |
| t: | | | | −1.383 | 0.151 | −0.300 | −2.968 | −2.916 | −4.077 | −2.570 | −2.290 | −4.262 | −2.853 |
| DF: | | | | 7 | 7 | 7 | 7 | 7 | 7 | 7 | 7 | 7 | 7 |
| P: | | | | 0.2093 | 0.8846 | 0.7729 | 0.0209 | 0.0225 | 0.0047 | 0.0370 | 0.0558 | 0.0037 | 0.0246 |
| Camptosar | 1.8 | | | 0.144 | 0.196 | 0.278 | 0.417 | 0.357 | 0.340 | 0.260 | 0.250 | 0.254 | 0.212 |
| huCBE11 | 2 | | | 0.101 | 0.137 | 0.322 | 0.458 | 0.497 | 0.542 | 0.524 | 0.541 | 0.485 | 0.541 |
| huCBE11 | 2 | Camptosar | 1.8 | 0.092 | 0.287 | 0.530 | 0.748 | 0.793 | 0.791 | 0.775 | 0.776 | 0.715 | 0.699 |
| Additive: | | | | 0.246 | 0.332 | 0.600 | 0.875 | 0.854 | 0.882 | 0.785 | 0.791 | 0.739 | 0.752 |
| Difference: | | | | −0.154 | −0.046 | −0.070 | −0.127 | −0.061 | −0.091 | −0.009 | −0.014 | −0.023 | −0.054 |
| t: | | | | −2.193 | −1.098 | −1.732 | −3.729 | −1.252 | −1.503 | −0.135 | −0.219 | −0.294 | −0.673 |
| DF: | | | | 7 | 7 | 7 | 7 | 7 | 7 | 7 | 7 | 7 | 7 |
| P: | | | | 0.0644 | 0.3086 | 0.1269 | 0.0074 | 0.2507 | 0.1765 | 0.8961 | 0.8329 | 0.7775 | 0.5224 |
| Camptosar | 3 | | | 0.098 | 0.327 | 0.466 | 0.593 | 0.521 | 0.463 | 0.406 | 0.381 | 0.368 | 0.335 |
| huCBE11 | 2 | | | 0.101 | 0.137 | 0.322 | 0.458 | 0.497 | 0.542 | 0.524 | 0.541 | 0.485 | 0.541 |
| huCBE11 | 2 | Camptosar | 3 | 0.108 | 0.377 | 0.682 | 0.852 | 0.883 | 0.897 | 0.888 | 0.886 | 0.859 | 0.852 |
| Additive: | | | | 0.199 | 0.464 | 0.789 | 1.051 | 1.018 | 1.005 | 0.930 | 0.922 | 0.853 | 0.875 |
| Difference: | | | | −0.091 | −0.087 | −0.107 | −0.198 | −0.136 | −0.109 | −0.042 | −0.036 | 0.006 | −0.023 |
| t: | | | | −1.081 | −1.585 | −2.808 | −6.581 | −4.138 | −2.642 | −0.927 | −0.719 | 0.141 | −0.479 |
| DF: | | | | 7 | 7 | 7 | 7 | 7 | 7 | 7 | 7 | 7 | 7 |
| P: | | | | 0.3156 | 0.1569 | 0.0262 | 0.0003 | 0.0044 | 0.0333 | 0.3847 | 0.4954 | 0.8916 | 0.6466 |
| Camptosar | 3 | | | 0.098 | 0.327 | 0.466 | 0.593 | 0.521 | 0.463 | 0.406 | 0.381 | 0.368 | 0.335 |
| huCBE11 | 20 | | | 0.053 | 0.087 | 0.263 | 0.435 | 0.504 | 0.576 | 0.539 | 0.543 | 0.525 | 0.538 |
| huCBE11 | 20 | Camptosar | 3 | 0.043 | 0.376 | 0.714 | 0.892 | 0.943 | 0.959 | 0.957 | 0.953 | 0.927 | 0.909 |

| | | | | | | | | | | |
|---|---|---|---|---|---|---|---|---|---|---|
| Additive: | 0.151 | 0.413 | 0.730 | 1.028 | 1.026 | 1.039 | 0.945 | 0.924 | 0.893 | 0.872 |
| Difference: | −0.108 | −0.037 | −0.016 | −0.136 | −0.083 | −0.080 | 0.013 | 0.029 | 0.034 | 0.037 |
| t: | −1.521 | −0.757 | −0.515 | −7.466 | −7.095 | −8.447 | 1.255 | 2.372 | 1.776 | 1.582 |
| DF: | 7 | 7 | 7 | 7 | 7 | 7 | 7 | 7 | 7 | 7 |
| P: | 0.1720 | 0.4737 | 0.6226 | 0.0001 | 0.0002 | 0.0001 | 0.2497 | 0.0494 | 0.1190 | 0.1576 |

Following observation of anti-tumor activity of separate and combined huCBE11 and Camptosar treatments and calculation of Fa values, the relationship between huCBE11 and Camptosar were then tested for synergy. Because treatment of mice bearing the KM-20L2 tumor with Camptosar alone produced dose-responsive antitumor efficacy, synergism of the huCBE11+Camptosar combination could be formally assessed by calculating the Combination Index (CI). Dose-effect relationships used in CI calculations are shown in Table 16 and the median effect doses are summarized in Table 17. Those doses used to assess synergistic drug action in the current study were given in a fixed ratio of 1:0.63 (mg/kg huCEB11:mg/kg Camptosar). This ratio was based on the ratio of the median effect doses for the 2 agents determined in the above-mentioned studies. Formal assessment of synergism employed calculation of the Combination Index (CI) using CalcuSyn V1.1 (Biosoft, Cambridge, UK) software for Windows-based dose-effect analysis. As described above, for treatments given in combination, a CI=1 indicates additive efficacy. CI<1 indicates synergism. CI>1 indicates antagonism. For treatments given in combination, a CI equal to 1 indicated additive efficacy. CI less than 1 indicated synergism. CI greater than 1 indicated antagonism. Through Combination Index testing of Camptosar and huCBE11, it was observed that fixed-ratio combination treatment of 1:0.63 (mg/kg huCEB11:mg/kg Camptosar) showed synergistic antitumor efficacy. Potency and shape of the dose-response relation for separate and combination treatments of Camptosar and huCBE11 are shown in Tables 18 and 19, respectively. The CIs calculated for the exact level of the experimental doses used in the combination treatment study are given in Table 19.

TABLE 16
huCBE11 and Camptosar Dose-Effect Relationships (Fa values) for Combination Index Calculation

| Day | | | | | | | | |
|---|---|---|---|---|---|---|---|---|
| 13 | 16 | 20 | 23 | 27 | 30 | 34 | 37 | 41 |

| | | | | |
|---|---|---|---|---|
| Treatment | Dose (mg/kg) | Cotreatment | Dose (mg/kg) | Fraction affected (Fa) |

Given Separately

| | | | | | | | | | | |
|---|---|---|---|---|---|---|---|---|---|---|
| huCBE11 | 0.2 | 0.003 | 0.125 | 0.249 | 0.294 | 0.377 | 0.369 | 0.345 | 0.372 | 0.381 |
| | 2.0 | 0.137 | 0.322 | 0.458 | 0.497 | 0.542 | 0.524 | 0.541 | 0.485 | 0.541 |
| | 20.0 | 0.086 | 0.264 | 0.435 | 0.504 | 0.576 | 0.539 | 0.543 | 0.525 | 0.538 |
| Camptosar | 1.8 | 0.196 | 0.278 | 0.417 | 0.357 | 0.340 | 0.260 | 0.250 | 0.254 | 0.212 |
| | 3.0 | 0.327 | 0.467 | 0.593 | 0.521 | 0.463 | 0.406 | 0.381 | 0.368 | 0.335 |
| | 6.0 | 0.360 | 0.594 | 0.781 | 0.759 | 0.701 | 0.607 | 0.564 | 0.495 | 0.444 |

Combined (1:0.63)

| | | | | | | | | | | | | |
|---|---|---|---|---|---|---|---|---|---|---|---|---|
| huCBE11 | 2.84 | Camptosar | 1.8 | 0.082 | 0.459 | 0.775 | 0.841 | 0.859 | 0.855 | 0.830 | 0.796 | 0.779 |
| | 4.74 | | 3.0 | 0.363 | 0.747 | 0.901 | 0.936 | 0.953 | 0.938 | 0.933 | 0.901 | 0.892 |
| | 9.48 | | 6.0 | 0.385 | 0.770 | 0.945 | 0.978 | 0.994 | 0.996 | 0.995 | 0.981 | 0.974 |

TABLE 17
huCBE11 and Camptosar: Median Effect Doses (mg/kg) for Combination
Index Calculation

| | huCBE11 | | | |
|---|---|---|---|---|
| | Given | | Camptosar | |
| | Separately | Combined (1:0.63) | Given Separately | Combined (1:0.63) |
| Day | Median Effect Dose (95% Confidence Interval) | | | |
| 13 | 174 | 10.7 | 12.4 | 6.7 |
| | (0-220250) | (3.5-32.7) | (3.2-47.7) | (2.2-20.6) |
| 16 | 981 | 2.6 | 4.0 | 1.6 |
| | (0-1300300) | (1.0-6.7) | (3.1-5.0) | (0.6-4.2) |
| 20 | 32.7 | 1.0 | 2.3 | 0.6 |
| | (0-3435) | (0.5-2.0) | (2.2-2.4) | (0.3-1.3) |
| 23 | 9.0 | 1.1 | 2.7 | 0.7 |
| | (0.7-115) | (0.9-1.3) | (2.6-2.9) | (0.6-0.8) |
| 27 | 2.1 | 1.5 | 3.2 | 1.0 |
| | (0.5-9) | (1.3-1.8) | (2.9-3.5) | (0.8-1.1) |
| 30 | 3.8 | 1.8 | 4.2 | 1.1 |
| | (0.6-24) | (1.1-2.7) | (4.0-4.3) | (0.7-1.7) |
| 34 | 3.6 | 1.8 | 4.7 | 1.1 |
| | (0.4-31) | (1.3-2.6) | (4.5-4.9) | (0.8-1.6) |
| 37 | 6.6 | 1.6 | 6.0 | 1.0 |
| | (1.9-22) | (1.2-2.1) | (5.1-6.9) | (0.8-1.3) |
| 41 | 3.0 | 1.5 | 7.3 | 1.0 |
| | (0.3-29) | (1.3-1.9) | (5.2-10.3) | (0.8-1.2) |

TABLE 18
huCBE11 and Camptosar (KM-20L2 Xenograft Model) Dose-Effect Curve Characteristics

| | | huCBE11 | | | Camptosar | | | huCBE11 + Camptosar | | |
|---|---|---|---|---|---|---|---|---|---|---|
| Day | Value | Slope | Y-Intercept | R | Slope | Y-Intercept | R | Slope | Y-Intercept | R |
| 13 | Mean | 0.75 | −1.67 | 0.7989 | 0.67 | −0.73 | 0.9021 | 1.53 | −1.57 | 0.8434 |
| | SEM | 0.56 | 0.49 | | 0.32 | 0.17 | | 0.98 | 0.72 | |
| 16 | Mean | 0.20 | −0.60 | 0.7325 | 1.09 | −0.65 | 0.9758 | 1.08 | −0.45 | 0.8642 |
| | SEM | 0.19 | 0.16 | | 0.24 | 0.13 | | 0.63 | 0.46 | |
| 20 | Mean | 0.18 | −0.28 | 0.8173 | 1.33 | −0.48 | 0.9998 | 1.31 | −0.01 | 0.9787 |
| | SEM | 0.13 | 0.11 | | 0.03 | 0.02 | | 0.27 | 0.20 | |
| 23 | Mean | 0.19 | −0.18 | 0.8795 | 1.45 | −0.64 | 0.9991 | 1.76 | −0.05 | 0.9982 |
| | SEM | 0.10 | 0.09 | | 0.06 | 0.03 | | 0.11 | 0.08 | |
| 27 | Mean | 0.18 | −0.06 | 0.9347 | 1.27 | −0.63 | 0.9954 | 2.76 | −0.50 | 0.9976 |
| | SEM | 0.07 | 0.06 | | 0.12 | 0.07 | | 0.19 | 0.14 | |
| 30 | Mean | 0.15 | −0.09 | 0.9026 | 1.23 | −0.76 | 0.9996 | 3.16 | −0.77 | 0.9815 |
| | SEM | 0.07 | 0.06 | | 0.04 | 0.02 | | 0.62 | 0.45 | |
| 34 | Mean | 0.18 | −0.10 | 0.8703 | 1.12 | −0.76 | 0.9995 | 3.12 | −0.81 | 0.9873 |
| | SEM | 0.10 | 0.09 | | 0.04 | 0.02 | | 0.50 | 0.37 | |
| 37 | Mean | 0.14 | −0.11 | 0.9627 | 0.87 | −0.68 | 0.9954 | 2.16 | −0.43 | 0.9940 |
| | SEM | 0.04 | 0.03 | | 0.08 | 0.05 | | 0.24 | 0.17 | |
| 41 | Mean | 0.14 | −0.07 | 0.8578 | 0.89 | −0.77 | 0.9847 | 1.97 | −0.37 | 0.9973 |
| | SEM | 0.08 | 0.07 | | 0.16 | 0.09 | | 0.15 | 0.11 | |

TABLE 19
huCBE11 and Camptosar (Cam): Combination Index (CI) for
Experimental Values

| | | | | Mechanism of Action | | | |
|---|---|---|---|---|---|---|---|
| | | | Fraction | Mutually | | | |
| | Dose (mg/kg) | | Affect- | Exclusive | | Nonexclusive | |
| Day | huCBE11 | Cam | ed | CI | Synergism | CI | Synergism |
| 13 | 2.84 | 1.8 | 0.082 | 5.842 | −−−− | 8.090 | −−−− |
| | 4.74 | 3.0 | 0.363 | 0.620 | +++ | 0.653 | +++ |
| | 9.48 | 6.0 | 0.385 | 1.078 | ± | 1.178 | − |
| 16 | 2.84 | 1.8 | 0.459 | 0.532 | +++ | 0.536 | +++ |
| | 4.74 | 3.0 | 0.747 | 0.279 | ++++ | 0.279 | ++++ |
| | 9.48 | 6.0 | 0.770 | 0.497 | +++ | 0.497 | +++ |
| 20 | 2.84 | 1.8 | 0.775 | 0.308 | +++ | 0.308 | +++ |
| | 4.74 | 3.0 | 0.901 | 0.248 | ++++ | 0.248 | ++++ |
| | 9.48 | 6.0 | 0.945 | 0.308 | +++ | 0.308 | +++ |
| 23 | 2.84 | 1.8 | 0.841 | 0.206 | ++++ | 0.206 | ++++ |
| | 4.74 | 3.0 | 0.936 | 0.170 | ++++ | 0.170 | ++++ |
| | 9.48 | 6.0 | 0.978 | 0.158 | ++++ | 0.158 | ++++ |
| 27 | 2.84 | 1.8 | 0.859 | 0.137 | ++++ | 0.137 | ++++ |
| | 4.74 | 3.0 | 0.953 | 0.088 | +++++ | 0.088 | +++++ |
| | 9.48 | 6.0 | 0.994 | 0.034 | +++++ | 0.034 | +++++ |
| 30 | 2.84 | 1.8 | 0.855 | 0.101 | ++++ | 0.101 | ++++ |
| | 4.74 | 3.0 | 0.938 | 0.078 | +++++ | 0.078 | +++++ |
| | 9.48 | 6.0 | 0.996 | 0.016 | +++++ | 0.016 | +++++ |
| 34 | 2.84 | 1.8 | 0.830 | 0.092 | +++++ | 0.092 | +++++ |
| | 4.74 | 3.0 | 0.933 | 0.061 | +++++ | 0.061 | +++++ |
| | 9.48 | 6.0 | 0.995 | 0.011 | +++++ | 0.011 | +++++ |
| 37 | 2.84 | 1.8 | 0.796 | 0.063 | +++++ | 0.063 | +++++ |
| | 4.74 | 3.0 | 0.901 | 0.040 | +++++ | 0.040 | +++++ |
| | 9.48 | 6.0 | 0.981 | 0.011 | +++++ | 0.011 | +++++ |
| 41 | 2.84 | 1.8 | 0.779 | 0.059 | +++++ | 0.059 | +++++ |
| | 4.74 | 3.0 | 0.892 | 0.038 | +++++ | 0.038 | +++++ |
| | 9.48 | 6.0 | 0.974 | 0.014 | +++++ | 0.014 | +++++ |

+++++ Very strong synergism
++++ Strong synergism
+++ Synergism
± Nearly additive
− Slight antagonism
−−−− Strong antagonism

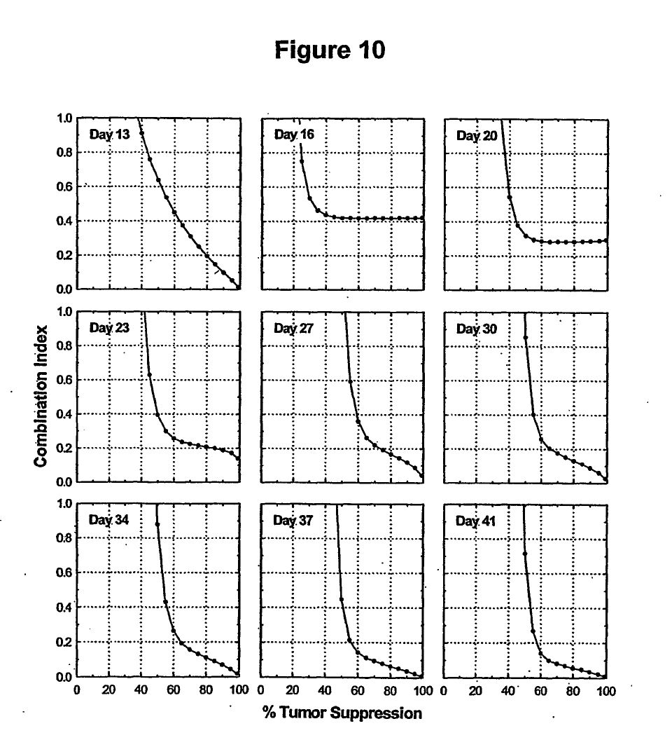

Because the current study employed drugs that are thought to have entirely independent modes of action, mutually nonexclusive CI values probably apply. Combination doses using 2.84 mg/kg huCBE11+1.8 mg/kg Camptosar, 4.74 mg/kg huCBE11+3 mg/kg Camptosar, and 9.48 mg/kg huCBE11+6 mg/kg Camptosar showed a synergistic effect on Days 16 to 41 (Table 19). Simulations of the CI over a range of dose levels for the combination are given in Tables 20 and 21 and overall interpretation of the degree of synergism or antagonism indicated by the CI is given in Table 3. Synergistic effects were present throughout the entire treatment period. CI as a function of percent tumor suppression is shown in FIG. 10. Synergism was most marked at levels of tumor suppression above 50%. Peak synergistic effects for the combination were shown on Day 16. The dose range that produced 20% to 80% tumor suppression combined the 2 drugs at dose levels between 1 and 100 mg/kg. In sum, fixed-ratio combination treatment (1:0.63) of huCBE11 plus Camptosar showed a synergistic antitumor effect.

TABLE 20
Combination Index Simulations: Mutually Exclusive Modes of Action

| | | Dose (mg/kg) | | | | Dose (mg/kg) | | | | Dose (mg/kg) | | |
|---|---|---|---|---|---|---|---|---|---|---|---|---|
| Fa | CI | huCBE11 | Cam | Symbol | CI | huCBE11 | Cam | Symbol | CI | huCBE11 | Cam | Symbol |
| | Day 13 | | | | Day 16 | | | | Day 20 | | | |
| 0.02 | 16 | 0.8 | 0.5 | −−−−− | 21000 | 0.07 | 0.05 | −−−−− | 2.8E+06 | 0.05 | 0.03 | −−−−− |
| 0.05 | 7 | 1.6 | 1.0 | −−−− | 440 | 0.2 | 0.1 | −−−−− | 32000 | 0.1 | 0.1 | −−−−− |
| 0.10 | 3.8 | 2.5 | 1.6 | −−−− | 21 | 0.3 | 0.2 | −−−−− | 954 | 0.2 | 0.1 | −−−−− |
| 0.15 | 2.56 | 3.4 | 2.2 | −−− | 3.6 | 0.5 | 0.3 | −−−− | 109 | 0.3 | 0.2 | −−−−− |
| 0.20 | 1.918 | 4.3 | 2.7 | −−− | 1.18 | 0.7 | 0.5 | − | 21 | 0.4 | 0.2 | −−−−− |
| 0.25 | 1.509 | 5.2 | 3.3 | −−− | 0.651 | 1.0 | 0.6 | +++ | 5.7 | 0.4 | 0.3 | −−−− |
| 0.30 | 1.224 | 6.1 | 3.9 | −− | 0.500 | 1.2 | 0.8 | +++ | 1.94 | 0.5 | 0.3 | −−− |
| 0.35 | 1.012 | 7.1 | 4.5 | ± | 0.449 | 1.5 | 0.9 | +++ | 0.847 | 0.6 | 0.4 | ++ |
| 0.40 | 0.848 | 8.2 | 5.2 | ++ | 0.430 | 1.8 | 1.1 | +++ | 0.486 | 0.7 | 0.5 | +++ |
| 0.45 | 0.715 | 9.4 | 5.9 | ++ | 0.423 | 2.2 | 1.4 | +++ | 0.358 | 0.9 | 0.5 | +++ |
| 0.50 | 0.605 | 11 | 7 | +++ | 0.419 | 2.6 | 1.6 | +++ | 0.310 | 1.0 | 0.6 | +++ |
| 0.55 | 0.512 | 12 | 8 | +++ | 0.418 | 3.2 | 2.0 | +++ | 0.292 | 1.2 | 0.7 | ++++ |
| 0.60 | 0.432 | 14 | 9 | +++ | 0.418 | 3.8 | 2.4 | +++ | 0.285 | 1.4 | 0.9 | ++++ |
| 0.65 | 0.362 | 16 | 10 | +++ | 0.418 | 4.6 | 2.9 | +++ | 0.283 | 1.6 | 1.0 | ++++ |
| 0.70 | 0.300 | 19 | 12 | ++++ | 0.418 | 5.7 | 3.6 | +++ | 0.282 | 1.9 | 1.2 | ++++ |
| 0.75 | 0.243 | 22 | 14 | ++++ | 0.418 | 7.2 | 4.5 | +++ | 0.283 | 2.4 | 1.5 | ++++ |
| 0.80 | 0.192 | 26 | 17 | ++++ | 0.418 | 9.4 | 5.9 | +++ | 0.284 | 2.9 | 1.8 | ++++ |
| 0.85 | 0.144 | 33 | 21 | ++++ | 0.419 | 13 | 8 | +++ | 0.285 | 3.8 | 2.4 | ++++ |
| 0.90 | 0.098 | 45 | 28 | +++++ | 0.419 | 20 | 13 | +++ | 0.287 | 5.4 | 3.4 | ++++ |
| 0.95 | 0.053 | 73 | 46 | +++++ | 0.420 | 40 | 25 | +++ | 0.290 | 10 | 6 | ++++ |
| 0.99 | 0.014 | 215 | 135 | +++++ | 0.422 | 181 | 114 | +++ | 0.296 | 34 | 21 | ++++ |
| | Day 23 | | | | Day 27 | | | | Day 30 | | | |
| 0.02 | 7.0E+06 | 0.12 | 0.07 | −−−−− | 7.4E+08 | 0.4 | 0.2 | −−−−− | 2.3E+10 | 0.5 | 0.3 | −−−−− |
| 0.05 | 9.0E+04 | 0.2 | 0.1 | −−−−− | 4.7E+06 | 0.5 | 0.3 | −−−−− | 5.8E+07 | 0.7 | 0.4 | −−−−− |
| 0.10 | 2893 | 0.3 | 0.2 | −−−−− | 88100 | 0.7 | 0.4 | −−−−− | 512000 | 0.9 | 0.6 | −−−−− |
| 0.15 | 346 | 0.4 | 0.3 | −−−−− | 7477 | 0.8 | 0.5 | −−−−− | 27400 | 1.0 | 0.6 | −−−−− |
| 0.20 | 70 | 0.5 | 0.3 | −−−−− | 1168 | 0.9 | 0.6 | −−−−− | 3021 | 1.1 | 0.7 | −−−−− |
| 0.25 | 19 | 0.6 | 0.4 | −−−−− | 252 | 1.0 | 0.6 | −−−−− | 489 | 1.2 | 0.8 | −−−−− |
| 0.30 | 6.1 | 0.7 | 0.4 | −−−− | 66 | 1.1 | 0.7 | −−−−− | 100 | 1.3 | 0.8 | −−−−− |
| 0.35 | 2.3 | 0.8 | 0.5 | −−− | 20 | 1.2 | 0.8 | −−−−− | 24 | 1.4 | 0.9 | −−−−− |
| 0.40 | 1.03 | 0.9 | 0.5 | ± | 6.6 | 1.3 | 0.8 | −−−− | 6.4 | 1.5 | 1.0 | −−−− |
| 0.45 | 0.553 | 1.0 | 0.6 | +++ | 2.4 | 1.4 | 0.9 | −−− | 2.0 | 1.6 | 1.0 | −−− |
| 0.50 | 0.366 | 1.1 | 0.7 | +++ | 1.02 | 1.5 | 1.0 | ± | 0.73 | 1.8 | 1.1 | ++ |
| 0.55 | 0.288 | 1.2 | 0.8 | ++++ | 0.525 | 1.6 | 1.0 | +++ | 0.370 | 1.9 | 1.2 | +++ |
| 0.60 | 0.253 | 1.4 | 0.9 | ++++ | 0.338 | 1.8 | 1.1 | +++ | 0.252 | 2.0 | 1.3 | ++++ |
| 0.65 | 0.235 | 1.5 | 1.0 | ++++ | 0.259 | 1.9 | 1.2 | ++++ | 0.203 | 2.1 | 1.3 | ++++ |
| 0.70 | 0.224 | 1.7 | 1.1 | ++++ | 0.219 | 2.1 | 1.3 | ++++ | 0.175 | 2.3 | 1.4 | ++++ |
| 0.75 | 0.216 | 2.0 | 1.3 | ++++ | 0.192 | 2.3 | 1.4 | ++++ | 0.153 | 2.5 | 1.6 | ++++ |
| 0.80 | 0.208 | 2.4 | 1.5 | ++++ | 0.168 | 2.5 | 1.6 | ++++ | 0.132 | 2.7 | 1.7 | ++++ |
| 0.85 | 0.199 | 2.9 | 1.8 | ++++ | 0.145 | 2.8 | 1.8 | ++++ | 0.111 | 3.0 | 1.9 | ++++ |
| 0.90 | 0.188 | 3.8 | 2.4 | ++++ | 0.119 | 3.4 | 2.1 | ++++ | 0.088 | 3.5 | 2.2 | +++++ |
| 0.95 | 0.172 | 5.7 | 3.6 | ++++ | 0.087 | 4.4 | 2.8 | +++++ | 0.061 | 4.4 | 2.8 | +++++ |
| 0.99 | 0.141 | 15 | 9 | ++++ | 0.043 | 8.0 | 5.1 | +++++ | 0.027 | 7.5 | 4.7 | +++++ |
| | Day 34 | | | | Day 37 | | | | Day 41 | | | |
| 0.02 | 5.5E+08 | 0.5 | 0.3 | −−−−− | 1.2E+11 | 0.3 | 0.2 | −−−−− | 1.2E+11 | 0.2 | 0.1 | −−−−− |
| 0.05 | 3.5E+06 | 0.7 | 0.4 | −−−−− | 1.7E+08 | 0.4 | 0.3 | −−−−− | 2.0E+08 | 0.3 | 0.2 | −−−−− |
| 0.10 | 64000 | 0.9 | 0.6 | −−−−− | 967000 | 0.6 | 0.4 | −−−−− | 1.3E+06 | 0.5 | 0.3 | −−−−− |
| 0.15 | 5409 | 1.0 | 0.7 | −−−−− | 39400 | 0.7 | 0.4 | −−−−− | 59000 | 0.6 | 0.4 | −−−−− |
| 0.20 | 842 | 1.2 | 0.7 | −−−−− | 3533 | 0.8 | 0.5 | −−−−− | 5687 | 0.8 | 0.5 | −−−−− |
| 0.25 | 182 | 1.3 | 0.8 | −−−−− | 483 | 1.0 | 0.6 | −−−−− | 824 | 0.9 | 0.6 | −−−−− |
| 0.30 | 48 | 1.4 | 0.9 | −−−−− | 85 | 1.1 | 0.7 | −−−−− | 153 | 1.0 | 0.6 | −−−−− |
| 0.35 | 14 | 1.5 | 0.9 | −−−−− | 18 | 1.2 | 0.7 | −−−−− | 33 | 1.1 | 0.7 | −−−−− |
| 0.40 | 4.8 | 1.6 | 1.0 | −−−− | 4.2 | 1.3 | 0.8 | −−−− | 8.0 | 1.3 | 0.8 | −−−− |
| 0.45 | 1.8 | 1.7 | 1.1 | −−− | 1.2 | 1.4 | 0.9 | − | 2.1 | 1.4 | 0.9 | −−− |
| 0.50 | 0.76 | 1.8 | 1.1 | ++ | 0.41 | 1.6 | 1.0 | +++ | 0.65 | 1.5 | 1.0 | +++ |
| 0.55 | 0.392 | 1.9 | 1.2 | +++ | 0.205 | 1.7 | 1.1 | ++++ | 0.251 | 1.7 | 1.1 | ++++ |
| 0.60 | 0.252 | 2.1 | 1.3 | ++++ | 0.141 | 1.9 | 1.2 | ++++ | 0.137 | 1.9 | 1.2 | ++++ |
| 0.65 | 0.189 | 2.2 | 1.4 | ++++ | 0.113 | 2.1 | 1.3 | ++++ | 0.099 | 2.1 | 1.3 | +++++ |
| 0.70 | 0.155 | 2.4 | 1.5 | ++++ | 0.094 | 2.3 | 1.5 | +++++ | 0.080 | 2.4 | 1.5 | +++++ |
| 0.75 | 0.131 | 2.6 | 1.6 | ++++ | 0.079 | 2.6 | 1.7 | +++++ | 0.068 | 2.7 | 1.7 | +++++ |
| 0.80 | 0.111 | 2.8 | 1.8 | ++++ | 0.065 | 3.0 | 1.9 | +++++ | 0.056 | 3.1 | 2.0 | +++++ |
| 0.85 | 0.090 | 3.2 | 2.0 | +++++ | 0.051 | 3.5 | 2.2 | +++++ | 0.046 | 3.7 | 2.3 | +++++ |
| 0.90 | 0.069 | 3.7 | 2.3 | +++++ | 0.037 | 4.4 | 2.8 | +++++ | 0.034 | 4.7 | 3.0 | +++++ |
| 0.95 | 0.045 | 4.7 | 2.9 | +++++ | 0.022 | 6.2 | 3.9 | +++++ | 0.022 | 6.9 | 4.3 | +++++ |
| 0.99 | 0.018 | 7.9 | 5.0 | +++++ | 0.007 | 13.2 | 8.3 | +++++ | 0.008 | 15.9 | 10.0 | +++++ |

TABLE 21
huCBE11 and Camptosar (Cam): Combination Index Simulations: Mutually Nonexclusive
(Totally Independent) Modes of Action

| | | Dose (mg/kg) | | | | Dose (mg/kg) | | | | Dose (mg/kg) | | |
|---|---|---|---|---|---|---|---|---|---|---|---|---|
| Fa | CI | huCBE11 | Cam | Symbol | CI | huCBE11 | Cam | Symbol | CI | huCBE11 | Cam | Symbol |
| | Day 13 | | | | Day 16 | | | | Day 20 | | | |
| 0.02 | 29 | 0.8 | 0.5 | −−−−− | 29600 | 0.07 | 0.05 | −−−−− | 3.5E+06 | 0.05 | 0.03 | −−−−− |
| 0.05 | 10 | 1.6 | 1.0 | −−−− | 622 | 0.2 | 0.1 | −−−−− | 40600 | 0.1 | 0.1 | −−−−− |
| 0.10 | 4.7 | 2.5 | 1.6 | −−−− | 30 | 0.3 | 0.2 | −−−−− | 1212 | 0.2 | 0.1 | −−−−− |
| 0.15 | 3.04 | 3.4 | 2.2 | −−− | 4.9 | 0.5 | 0.3 | −−−− | 138 | 0.3 | 0.2 | −−−−− |
| 0.20 | 2.20 | 4.3 | 2.7 | −−− | 1.50 | 0.7 | 0.5 | −−− | 27 | 0.4 | 0.2 | −−−−− |
| 0.25 | 1.69 | 5.2 | 3.3 | −−− | 0.749 | 1.0 | 0.6 | ++ | 7.2 | 0.4 | 0.3 | −−−− |
| 0.30 | 1.35 | 6.1 | 3.9 | −− | 0.535 | 1.2 | 0.8 | +++ | 2.41 | 0.5 | 0.3 | −−− |
| 0.35 | 1.099 | 7.1 | 4.5 | ± | 0.463 | 1.5 | 0.9 | +++ | 1.005 | 0.6 | 0.4 | ± |
| 0.40 | 0.910 | 8.2 | 5.2 | ± | 0.436 | 1.8 | 1.1 | +++ | 0.544 | 0.7 | 0.5 | +++ |
| 0.45 | 0.760 | 9.4 | 5.9 | ++ | 0.425 | 2.2 | 1.4 | +++ | 0.380 | 0.9 | 0.5 | +++ |
| 0.50 | 0.639 | 11 | 7 | +++ | 0.420 | 2.6 | 1.6 | +++ | 0.318 | 1.0 | 0.6 | +++ |
| 0.55 | 0.537 | 12 | 8 | +++ | 0.419 | 3.2 | 2.0 | +++ | 0.295 | 1.2 | 0.7 | ++++ |
| 0.60 | 0.450 | 14 | 9 | +++ | 0.418 | 3.8 | 2.4 | +++ | 0.286 | 1.4 | 0.9 | ++++ |
| 0.65 | 0.375 | 16 | 10 | +++ | 0.418 | 4.6 | 2.9 | +++ | 0.283 | 1.6 | 1.0 | ++++ |
| 0.70 | 0.309 | 19 | 12 | +++ | 0.418 | 5.7 | 3.6 | +++ | 0.283 | 1.9 | 1.2 | ++++ |
| 0.75 | 0.250 | 22 | 14 | ++++ | 0.418 | 7.2 | 4.5 | +++ | 0.283 | 2.4 | 1.5 | ++++ |
| 0.80 | 0.196 | 26 | 17 | ++++ | 0.418 | 9.4 | 5.9 | +++ | 0.284 | 2.9 | 1.8 | ++++ |
| 0.85 | 0.146 | 33 | 21 | ++++ | 0.419 | 13 | 8 | +++ | 0.285 | 3.8 | 2.4 | ++++ |
| 0.90 | 0.099 | 45 | 28 | +++++ | 0.419 | 20 | 13 | +++ | 0.287 | 5.4 | 3.4 | ++++ |
| 0.95 | 0.053 | 73 | 46 | +++++ | 0.420 | 40 | 25 | +++ | 0.290 | 10 | 6 | ++++ |
| 0.99 | 0.014 | 215 | 135 | +++++ | 0.422 | 181 | 114 | +++ | 0.296 | 34 | 21 | ++++ |
| | Day 23 | | | | Day 27 | | | | Day 30 | | | |
| 0.02 | 9.7E+06 | 0.12 | 0.07 | −−−−− | 1.9E+09 | 0.4 | 0.2 | −−−−− | 6.6E+10 | 0.5 | 0.3 | −−−−− |
| 0.05 | 1.2E+05 | 0.2 | 0.1 | −−−−− | 9.8E+06 | 0.5 | 0.3 | −−−−− | 1.3E+08 | 0.7 | 0.4 | −−−−− |
| 0.10 | 3824 | 0.3 | 0.2 | −−−−− | 156000 | 0.7 | 0.4 | −−−−− | 917000 | 0.9 | 0.6 | −−−−− |
| 0.15 | 451 | 0.4 | 0.3 | −−−−− | 12200 | 0.8 | 0.5 | −−−−− | 44600 | 1.0 | 0.6 | −−−−− |
| 0.20 | 90 | 0.5 | 0.3 | −−−−− | 1805 | 0.9 | 0.6 | −−−−− | 4615 | 1.1 | 0.7 | −−−−− |
| 0.25 | 24 | 0.6 | 0.4 | −−−−− | 374 | 1.0 | 0.6 | −−−−− | 713 | 1.2 | 0.8 | −−−−− |
| 0.30 | 7.7 | 0.7 | 0.4 | −−−− | 95 | 1.1 | 0.7 | −−−−− | 140 | 1.3 | 0.8 | −−−−− |
| 0.35 | 2.9 | 0.8 | 0.5 | −−− | 28 | 1.2 | 0.8 | −−−−− | 32 | 1.4 | 0.9 | −−−−− |
| 0.40 | 1.23 | 0.9 | 0.5 | −− | 8.9 | 1.3 | 0.8 | −−−− | 8.4 | 1.5 | 1.0 | −−−− |
| 0.45 | 0.629 | 1.0 | 0.6 | +++ | 3.1 | 1.4 | 0.9 | −−− | 2.4 | 1.6 | 1.0 | −−− |
| 0.50 | 0.395 | 1.1 | 0.7 | +++ | 1.24 | 1.5 | 1.0 | −− | 0.85 | 1.8 | 1.1 | ++ |
| 0.55 | 0.299 | 1.2 | 0.8 | ++++ | 0.593 | 1.6 | 1.0 | +++ | 0.401 | 1.9 | 1.2 | +++ |
| 0.60 | 0.257 | 1.4 | 0.9 | ++++ | 0.359 | 1.8 | 1.1 | +++ | 0.259 | 2.0 | 1.3 | ++++ |
| 0.65 | 0.237 | 1.5 | 1.0 | ++++ | 0.265 | 1.9 | 1.2 | ++++ | 0.205 | 2.1 | 1.3 | ++++ |
| 0.70 | 0.225 | 1.7 | 1.1 | ++++ | 0.221 | 2.1 | 1.3 | ++++ | 0.176 | 2.3 | 1.4 | ++++ |
| 0.75 | 0.216 | 2.0 | 1.3 | ++++ | 0.192 | 2.3 | 1.4 | ++++ | 0.153 | 2.5 | 1.6 | ++++ |
| 0.80 | 0.208 | 2.4 | 1.5 | ++++ | 0.168 | 2.5 | 1.6 | ++++ | 0.132 | 2.7 | 1.7 | ++++ |
| 0.85 | 0.199 | 2.9 | 1.8 | ++++ | 0.145 | 2.8 | 1.8 | ++++ | 0.111 | 3.0 | 1.9 | ++++ |
| 0.90 | 0.188 | 3.8 | 2.4 | ++++ | 0.119 | 3.4 | 2.1 | ++++ | 0.088 | 3.5 | 2.2 | +++++ |
| 0.95 | 0.172 | 5.7 | 3.6 | ++++ | 0.087 | 4.4 | 2.8 | +++++ | 0.061 | 4.4 | 2.8 | +++++ |
| 0.99 | 0.141 | 15 | 9 | ++++ | 0.043 | 8.0 | 5.1 | +++++ | 0.027 | 7.5 | 4.7 | +++++ |
| | Day 34 | | | | Day 37 | | | | Day 41 | | | |
| 0.02 | 1.8E+09 | 0.5 | 0.3 | −−−−− | 4.1E+11 | 0.3 | 0.2 | −−−−− | 2.9E+11 | 0.2 | 0.1 | −−−−− |
| 0.05 | 8.0E+06 | 0.7 | 0.4 | −−−−− | 3.9E+08 | 0.4 | 0.3 | −−−−− | 3.6E+08 | 0.3 | 0.2 | −−−−− |
| 0.10 | 1.2E+05 | 0.9 | 0.6 | −−−−− | 1.7E+06 | 0.6 | 0.4 | −−−−− | 2.0E+06 | 0.5 | 0.3 | −−−−− |
| 0.15 | 8938 | 1.0 | 0.7 | −−−−− | 61000 | 0.7 | 0.4 | −−−−− | 81800 | 0.6 | 0.4 | −−−−− |
| 0.20 | 1293 | 1.2 | 0.7 | −−−−− | 5060 | 0.8 | 0.5 | −−−−− | 7463 | 0.8 | 0.5 | −−−−− |
| 0.25 | 264 | 1.3 | 0.8 | −−−−− | 654 | 1.0 | 0.6 | −−−−− | 1039 | 0.9 | 0.6 | −−−−− |
| 0.30 | 66 | 1.4 | 0.9 | −−−−− | 110 | 1.1 | 0.7 | −−−−− | 187 | 1.0 | 0.6 | −−−−− |
| 0.35 | 19 | 1.5 | 0.9 | −−−−− | 22 | 1.2 | 0.7 | −−−−− | 39 | 1.1 | 0.7 | −−−−− |
| 0.40 | 6.1 | 1.6 | 1.0 | −−−− | 5.1 | 1.3 | 0.8 | −−−− | 9.3 | 1.3 | 0.8 | −−−− |
| 0.45 | 2.2 | 1.7 | 1.1 | −−− | 1.3 | 1.4 | 0.9 | −− | 2.4 | 1.4 | 0.9 | −−− |
| 0.50 | 0.88 | 1.8 | 1.1 | + | 0.45 | 1.6 | 1.0 | +++ | 0.72 | 1.5 | 1.0 | ++ |
| 0.55 | 0.430 | 1.9 | 1.2 | +++ | 0.214 | 1.7 | 1.1 | ++++ | 0.267 | 1.7 | 1.1 | ++++ |
| 0.60 | 0.263 | 2.1 | 1.3 | ++++ | 0.143 | 1.9 | 1.2 | ++++ | 0.141 | 1.9 | 1.2 | ++++ |
| 0.65 | 0.193 | 2.2 | 1.4 | ++++ | 0.113 | 2.1 | 1.3 | ++++ | 0.099 | 2.1 | 1.3 | +++++ |
| 0.70 | 0.156 | 2.4 | 1.5 | ++++ | 0.094 | 2.3 | 1.5 | +++++ | 0.081 | 2.4 | 1.5 | +++++ |
| 0.75 | 0.132 | 2.6 | 1.6 | ++++ | 0.079 | 2.6 | 1.7 | +++++ | 0.068 | 2.7 | 1.7 | +++++ |
| 0.80 | 0.111 | 2.8 | 1.8 | ++++ | 0.065 | 3.0 | 1.9 | +++++ | 0.056 | 3.1 | 2.0 | +++++ |
| 0.85 | 0.090 | 3.2 | 2.0 | +++++ | 0.051 | 3.5 | 2.2 | +++++ | 0.046 | 3.7 | 2.3 | +++++ |
| 0.90 | 0.069 | 3.7 | 2.3 | +++++ | 0.037 | 4.4 | 2.8 | +++++ | 0.034 | 4.7 | 3.0 | +++++ |
| 0.95 | 0.045 | 4.7 | 2.9 | +++++ | 0.022 | 6.2 | 3.9 | +++++ | 0.022 | 6.9 | 4.3 | +++++ |
| 0.99 | 0.018 | 7.9 | 5.0 | +++++ | 0.007 | 13.2 | 8.3 | +++++ | 0.008 | 15.9 | 10.0 | +++++ |

In sum, the combination treatment of the LTβ receptor-activating mAb huCBE11 and the chemotherapeutic agent Camptosar in athymic nude mice implanted subcutaneously with KM-20L2 human colorectal adenocarcinoma showed significantly improved results in comparison to huCBE11 and Camptosar administered alone. Strikingly, the effect of fixed ratio 1:0.63 (mg/kg hCEB11:mg/kg Camptosar) combination treatment with huCBE11 and Camptosar was determined to be synergistic by combination index analysis, similar to the results of combination analysis for the WiDr murine model with the same compounds.

### Example 4

### Antitumor Efficacy of LTβR Agonist in Combination with Nucleoside Analog Chemotherapeutic Agent

A. Antitumor Efficacy of Combination huCBE11/emcitabine Therapy in WiDr Xenograft Murine Model

In order to determine whether administration of a nucleoside analog chemotherapeutic agent, e.g., gemcitabine, in combination with huCBE11 has supra-additive, e.g., synergistic, antitumor activity, gemcitabine was administered in combination with huCBE11 using the WiDr murine model.

A dose ranging study was performed to determine the appropriate gemcitabine dose(s) for studying the combined antitumor effects of gemcitabine and huCBE11. Mean (± standard error of the mean [SEM]) tumor weights for 4 gemcitabine groups (140, 100, 50, and 25 mg/kg) and saline control groups were measured over the course of the dosing study (Days 0 to 42). Tumor take rate was >98% on implantation, and 56 mice within a tight size range were selected to initiate treatments. Significant inhibition of tumor growth was observed on Day 42 in the gemcitabine 140 mg/kg (P=0.001), 100 mg/kg (P=0.0002), 50 mg/kg (P=0.008), and 25 mg/kg (P=0.006) groups compared with the saline control group. Inhibition was first evident in these dose groups by Days 11 to 14. Thus, all doses of gemcitabine studied (140, 100, 50, and 25 mg/kg) significantly (P<0.01) inhibited tumor growth compared with the saline control in athymic nude mice with pre-established WiDr human colorectal tumors.

In a separate study, effects of gemcitibine doses of 50, 25, 12.5, and 6.25 mg/kg were examined. Tumor take rate was 100% on implantation, and 180 mice within a tight size range were selected to initiate treatments. Significant inhibition of tumor growth was observed on Day 41 (end of study) in the gemcitabine 50 mg/kg (P<0.001), 25 mg/kg (P<0.001), and 6.25 mg/kg (P<0.05) groups compared with the vehicle control group. Inhibition was evident in these groups by Day 10. No significant inhibition was seen in the 12.5 mg/kg group on Day 41. The % T/C fell below 42% on Days 17 to 28 in the gemcitabine 50 mg/kg group and on Days 21 and 28 in the gemcitabine 25 mg/kg group. Apart from these timepoints, responses for administration of gemcitabine alone did not exceed the National Cancer Institute (NCI) activity criteria (percent test/control [% T/C] at or below 42), with all gemcitabine treatments examined deemed to be inactive by the NCI guidelines on Day 41 (end of study). In sum, gemcitabine doses of 50 mg/kg to 6.25 mg/kg were determined to be inactive in the WiDr model at the end of the second study on Day 41 based on the NCI activity criteria (% T/C of 42 or less). Significant inhibition of tumor growth was observed on Day 41 in the gemcitabine 50 mg/kg (P<0.001), 25 mg/kg (P<0.001), and 6.25 mg/kg (P<0.05) groups compared with the saline control group.

In a parallel study, huCBE11 activity was also examined in the WiDr human colorectal adenocarcinoma xenograft model. Three groups administered increasing doses (5, 50 and 100 μg) of gemcitabine and a saline control group were assayed for anti-tumor activity. Tumor growth was significantly (P<0.001) decreased on Day 41 in the huCBE11 50 μg and 100 μg groups compared with the vehicle control group. This decrease was evident by Day 14. Treatment with huCBE11 5 μg did not significantly inhibit tumor growth. The % T/C fell to 40.0% on Day 41 in the huCBE11 100 μg and to 43.5% on Day 41 in the 50 μg group. Thus, huCBE11 100 μg was determined to be active in the WiDr model based on the NCI activity criteria (% T/C of 42 or less).

The combination anti-tumor effect of huCBE11 and gemcitabine in the WiDr human colorectal adenocarcinoma xenograft model was also examined. Combination treatment of huCBE11 100 μg and gemcitabine 25 mg/kg (FIG. 2) or 12.5 mg/kg produced significant (P<0.01) inhibition of tumor growth in athymic nude mice on Day 41 compared with huCBE11 100 μg alone. The combination treatment of huCBE11 50 μg and gemcitabine 25 mg/kg or 12.5 mg/kg produced significant inhibition (P<0.01 and P<0.05, respectively) in tumor growth compared with huCBE11 50 μg alone. This inhibition was consistently apparent in all groups by Day 17, following 2 doses of huCBE11 and after all doses of gemcitabine had been administered. This effect continued throughout the course of the study. The % T/C was below 42% from Day 17 or 21 to 41 in all of these huCBE11 plus gemcitabine combination groups. The lowest % T/C values were observed on Day 41 (range across dose groups: 20.1-26.8%).

In addition, significant inhibition of tumor growth was observed at the end of study in the huCBE11 plus gemcitabine combination groups 88 μg/50 mg/kg (P<0.05), 44 μg/25 mg/kg (P<0.05), 22 μg/12.5 mg/kg (P<0.01), 11 μg/6.25 mg/kg (P=0.01). This inhibition became evident between Days 14 and 17. The % T/C was below 42% from Days 14 or 17 to 41 in the huCBE11 88 μg plus gemcitabine 50 mg/kg (low of 19.6%) and the huCBE11 44 μg plus gemcitabine 25 mg/kg (low of 24.5%) groups. The % T/C was at or below 42% from Days 28 to 41 in the huCBE11 22 μg plus gemcitabine 12.5 mg/kg (low of 37.9%) group. The lowest % T/C in the huCBE11 11 μg plus gemcitabine 6.25 mg/kg was 42.6% on Day 41. All but the lowest dose combination of hCBE11 plus gemcitabine had % T/C at or below 42% on Day 41 (range across dose groups: 20.1%-38.1%). Thus, combination treatment of hCBE11 plus gemcitabine was determined to be active in the WiDr model based on the NCI activity criteria (% T/C of 42 or less).

To examine whether huCBE11 and gemcitabine could act synergistically, supra-additive studies were performed using nude athymic mice implanted with WiDr human colon adenocarcinomas in a tumor growth model as described above. Doses of 50, 25, 12.5, and 6.25 mg/kg gemcitabine were chosen, as well as doses of 100, 88, 50, 44, 22, and 11 μg huCBE11 were chosen for these huCBE11+gemcitabine combination studies. All tumor data used to calculate Fa values were taken at day 28. Antitumor efficacy was determined by comparing each treatment group's tumor volume with the control group's tumor volume. Mean tumor volume decrease was calculated as the difference between control group and treatment group mean tumor volume. The fractional inhibition of tumor volume, Fraction affected (Fa), was calculated by dividing treatment group mean tumor volume decrease by control group mean tumor volume. An Fa of 1.000 would indicate complete inhibition of the tumor. Table 22 shows the dose-effect relationships for separate and combination treatments. The Fa values obtained were then used for assessment of synergy for combination huCBE11 and gemcitabine treatment.

TABLE 22
Dose-Effect Relationships for Separate and Combination Treatments of Gemcitabine
and huCBE11

| | | | | | | Tumor | Volume | |
|---|---|---|---|---|---|---|---|---|
| Treatment | Dose | Units | Cotreatment | Dose | Units | Volume | Decrease | Fa |
| Control | | | | | | 803.2 | 0.0 | 0.000 |
| Gemcitabine | 6.25 | mg/kg | | | | 545.6 | 257.6 | 0.321 |
| Gemcitabine | 12.5 | mg/kg | | | | 479.5 | 323.7 | 0.403 |
| Gemcitabine | 25 | mg/kg | | | | 318.9 | 484.3 | 0.603 |
| Gemcitabine | 50 | mg/kg | | | | 326.0 | 477.2 | 0.594 |
| huCBE11 | 5 | μg | | | | 688.4 | 114.8 | 0.143 |
| huCBE11 | 50 | μg | | | | 448.0 | 355.2 | 0.442 |
| huCBE11 | 100 | μg | | | | 386.1 | 417.1 | 0.519 |
| huCBE11 | 11 | μg | Gemcitabine | 6.25 | mg/kg | 342.7 | 460.5 | 0.573 |
| huCBE11 | 22 | μg | Gemcitabine | 12.5 | mg/kg | 304.5 | 498.7 | 0.621 |
| huCBE11 | 44 | μg | Gemcitabine | 25 | mg/kg | 196.9 | 606.3 | 0.755 |
| huCBE11 | 88 | μg | Gemcitabine | 50 | mg/kg | 157.8 | 645.4 | 0.804 |
| huCBE11 | 50 | μg | Gemcitabine | 12.5 | mg/kg | 206.9 | 596.3 | 0.742 |
| huCBE11 | 50 | μg | Gemcitabine | 25 | mg/kg | 171.8 | 631.4 | 0.786 |
| huCBE11 | 100 | μg | Gemcitabine | 12.5 | mg/kg | 217.0 | 586.2 | 0.730 |
| huCBE11 | 100 | μg | Gemcitabine | 25 | mg/kg | 196.5 | 606.7 | 0.755 |

Using the tumor weight data obtained from these combination treatment studies, statistical comparisons were performed to determine whether the combination of huCBE11 plus gemcitabine was synergistic in its mode of action. Because treatment of mice bearing the WiDr tumor with gemcitabine alone produced dose-responsive antitumor efficacy, synergism of the huCBE11+gemcitabine combination could be formally assessed by calculating the Combination Index (CI) (Chou, 1984). Formal assessment of synergism employed calculation of the Combination Index (C.I.) using CalcuSyn V1.1 (Biosoft, Cambridge, UK) software for Windows-based dose-effect analysis. As described above, for treatments given in combination, a C.I.=1 indicates additive efficacy. C.I.<1 indicates synergism. C.I.>1 indicates antagonism. Those doses used to assess synergistic drug action in the current study were given in a fixed ratio of 0.568:1 (mg/kg gemcitabine:μg huCBE11). This ratio was based on the ratio of the median effect doses for the 2 agents determined in previous pilot studies. Formal assessment of synergism employed calculation of the Combination Index (CI) using CalcuSyn V1.1 (Biosoft, Cambridge, UK) software for Windows-based dose-effect analysis. For treatments given in combination, a CI equal to 1 indicated additive efficacy. CI less than 1 indicated synergism. CI greater than 1 indicated antagonism. Dose-effect relationships used in CI calculations are shown in Table 23.

TABLE 23
Dose-Effect Relationships of huCBE11 and Gemcitabine for Synergism
Calculations

| Given Separately | | | | Combined (0.568:1) | | |
|---|---|---|---|---|---|---|
| Gemcitabine | | huCBE11 | | Gemcitabine | huCBE11 | |
| Dose | Fraction | Dose | Fraction | Dose | Dose | Fraction |
| (mg/kg) | Affected | (μg) | Affected | (mg/kg) | (μg) | Affected |
| 6.25 | 0.321 | 5 | 0.143 | 6.25 | 11 | 0.573 |
| 12.5 | 0.403 | 50 | 0.442 | 12.5 | 22 | 0.621 |
| 25 | 0.603 | 100 | 0.519 | 25 | 44 | 0.755 |
| | | | | 50 | 88 | 0.804 |

Potency and shape of the dose-response relation for separate and combination treatments of gemcitabine and huCBE11 are shown in Tables 24 and 25, respectively. The CI calculated for the exact level of the experimental doses used in this study are given in Table 26.

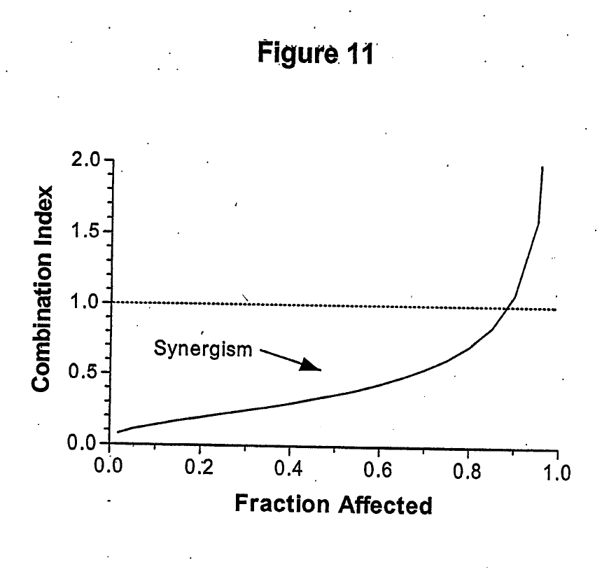

Because the current study employed drugs that are thought to have entirely independent modes of action, mutually nonexclusive CI values probably apply. Combination doses using 6.25 mg/kg gemcitabine+11.0 μg huCBE11, 12.5 mg/kg gemcitabine+22.0 μg huCBE11, 25 mg/kg gemcitabine+44 μg huCBE11, and 50 mg/kg gemcitabine+88 μg huCBE11 showed a synergistic effect. Simulations of the CI over a range of dose levels for the combination are given in Table 27 and overall interpretation of the degree of synergism or antagonism indicated by the CI is given in Table 3. Combination doses ranging from 0.005 mg/kg gemcitabine+0.008 μg huCBE11 (giving 2% inhibition of tumor volume) to 85 mg/kg gemcitabine+150 μg huCBE11 (giving an 85% inhibition of tumor volume) showed a synergistic effect. Thus, the fixed ratio combination treatment of 0.568:1 gemcitabine:huCBE11 showed synergistic antitumor efficacy. CI as a function of fraction affected is shown in FIG. 11.

TABLE 24
Determination of Synergism: Median Effect Doses for Synergism of
huCBE11 and Gemcitabine

| | | Median Effect Dose | |
|---|---|---|---|
| | | (95% Confidence Interval) | |
| Agent | Dose Units | Given Separately | Combined (0.568:1) |
| Gemcitabine | mg/kg | 16.7 | 4.2 |
| | | (12.6-22.0) | (2.6-6.6) |
| huCBE11 | μg | 81.1 | 7.3 |
| | | (65.2-100.8) | (4.6-11.6) |

TABLE 25
Determination of Synergism: Dose-Response Curve Characteristics for
Separate and Combination Treatments

| | | | |
|---|---|---|---|
| Value | Slope | Y-Intercept | R |

Gemcitabine

| | | | |
|---|---|---|---|
| Mean | 0.842 | −1.028 | 0.9756 |
| SEM | 0.189 | 0.213 | |

huCBE11

| | | | |
|---|---|---|---|
| Mean | 0.636 | −1.215 | 0.9977 |
| SEM | 0.043 | 0.068 | |

Gemcitabine + huCBE11

| | | | |
|---|---|---|---|
| Mean | 0.575 | −0.356 | 0.9804 |
| SEM | 0.082 | 0.106 | |

TABLE 26
Determination of Synergism: Combination Indices Calculated for
Experimental Values Obtained for huCBE11 + Gemcitabine
Treatment

| | | | Mechanisms of Action | | | |
|---|---|---|---|---|---|---|
| Gemcitabine | huCBE11 | Fraction | Mutually | | | |
| Dose | Dose | Affect- | Exclusive | | Nonexclusive | |
| (mg/kg) | (μg) | ed | CI | Synergism | CI | Synergism |
| 6.25 | 11 | 0.573 | 0.350 | +++ | 0.373 | +++ |
| 12.5 | 22 | 0.621 | 0.543 | +++ | 0.595 | +++ |
| 25 | 44 | 0.755 | 0.487 | +++ | 0.524 | +++ |
| 50 | 88 | 0.804 | 0.680 | +++ | 0.746 | ++ |

++ moderate synergism
+++ synergism

TABLE 27
Determination of Synergism: Combination Index (CI) Simulations

| | | | | |
|---|---|---|---|---|
| | | Gemcitabine | huCBE11 | |
| Fa | CI | (mg/kg) | (μg) | Symbol |

Mutually Exclusive Mechanisms of Action

| | | | | |
|---|---|---|---|---|
| 0.02 | 0.076 | 0.005 | 0.008 | +++++ |
| 0.05 | 0.104 | 0.025 | 0.044 | ++++ |
| 0.10 | 0.137 | 0.091 | 0.160 | ++++ |
| 0.15 | 0.163 | 0.2 | 0.4 | ++++ |
| 0.20 | 0.188 | 0.4 | 0.7 | ++++ |
| 0.25 | 0.211 | 0.6 | 1.1 | ++++ |
| 0.30 | 0.235 | 1.0 | 1.7 | ++++ |
| 0.35 | 0.259 | 1.4 | 2.5 | ++++ |
| 0.40 | 0.284 | 2.1 | 3.6 | ++++ |
| 0.45 | 0.311 | 2.9 | 5.2 | +++ |
| 0.50 | 0.340 | 4.2 | 7.3 | +++ |
| 0.55 | 0.373 | 5.9 | 10.4 | +++ |
| 0.60 | 0.409 | 8.4 | 14.8 | +++ |
| 0.65 | 0.452 | 12.2 | 21.5 | +++ |
| 0.70 | 0.503 | 18.2 | 32.0 | +++ |
| 0.75 | 0.567 | 28.2 | 49.5 | +++ |
| 0.80 | 0.652 | 46.4 | 81.7 | +++ |
| 0.85 | 0.773 | 85.1 | 149.8 | ++ |
| 0.90 | 0.972 | 190.4 | 335.1 | ± |
| 0.95 | 1.419 | 698.6 | 1229.6 | −− |
| 0.99 | 3.357 | 12347.0 | 21731.0 | −−−− |

Nonexclusive (Totally Independent Modes of Action)

| | | | | |
|---|---|---|---|---|
| 0.02 | 0.077 | 0.005 | 0.008 | +++++ |
| 0.05 | 0.107 | 0.025 | 0.044 | ++++ |
| 0.10 | 0.141 | 0.091 | 0.160 | ++++ |
| 0.15 | 0.170 | 0.2 | 0.4 | ++++ |
| 0.20 | 0.196 | 0.4 | 0.7 | ++++ |
| 0.25 | 0.222 | 0.6 | 1.1 | ++++ |
| 0.30 | 0.247 | 1.0 | 1.7 | ++++ |
| 0.35 | 0.273 | 1.4 | 2.5 | ++++ |
| 0.40 | 0.301 | 2.1 | 3.6 | +++ |
| 0.45 | 0.331 | 2.9 | 5.2 | +++ |
| 0.50 | 0.363 | 4.2 | 7.3 | +++ |
| 0.55 | 0.399 | 5.9 | 10.4 | +++ |
| 0.60 | 0.440 | 8.4 | 14.8 | +++ |
| 0.65 | 0.487 | 12.2 | 21.5 | +++ |
| 0.70 | 0.545 | 18.2 | 32.0 | +++ |
| 0.75 | 0.617 | 28.2 | 49.5 | +++ |
| 0.80 | 0.713 | 46.4 | 81.7 | ++ |
| 0.85 | 0.851 | 85.1 | 149.8 | + |
| 0.90 | 1.082 | 190.4 | 335.1 | ± |
| 0.95 | 1.608 | 698.6 | 1229.6 | −−− |
| 0.99 | 3.977 | 12347.0 | 21731.0 | −−−− |

Those doses used to assess drug potentiation in the current study were not restricted to fixed-ratio combination. Testing for statistically significant potentiation required the calculation of Fa for each animal. Individual tumor volumes (Table 43) were used to calculate fractional inhibition of tumor volume (Fa) for each animal (Table 44). Fa was calculated as (control group mean tumor volume−individual animal tumor volume)÷control group mean tumor volume. The expected additive Fa for a combination treatment was taken to be the sum of mean Fa's from groups receiving either element of the combination. The difference between a combination treatment's actual efficacy and that which would be expected if the treatments were merely additive was also calculated (Table 44). A two-tailed one-sample t-test was used to determine whether the combination treatment produced a mean Fa that was statistically significantly different from the expected additive value (Table 44).

Because the current study employs drugs that are thought to have entirely independent modes of action, mutually nonexclusive C.I. values probably apply. Combination doses using 6.25 mg/kg gemcitabine+11 μg huCBE11, 12.5 mg/kg gemcitabine+22 μg huCBE11, 25 mg/kg gemcitabine+44 μg huCBE11, and 50 mg/kg gemcitabine+88 μg huCBE11 showed a synergistic effect. Combination doses ranging from 0.005 mg/kg gemcitabine+0.008 μg huCBE11 (giving 2% inhibition of tumor volume) to 85 mg/kg gemcitabine+150 μg huCBE11 (giving an 85% inhibition of tumor volume) showed a synergistic effect. Overall interpretation of the degree of synergism or antagonism indicated by the C.I. is given in Table 15. When combined with 50 or 100 μg doses of huCBE11, gemcitabine doses of either 12.5 or 25 mg/kg produced effects that were statistically significantly less than additive (Table 44).

TABLE 43
Individual Tumor Volumes

| | | | | | | | | hCBE | hCBE | hCBE | hCBE |
|---|---|---|---|---|---|---|---|---|---|---|---|
| | | Gem | Gem | | | hCBE | hCBE | 50 + Gem | 50 + Gem | 100 + Gem | 100 + Gem |
| | Control | 6.25 | 12.5 | Gem 25 | Gem 50 | 50 | 100 | 12.5 | 25 | 12.5 | 25 |
| | 854.1 | 777.9 | 295.5 | 238.8 | 181.8 | 606.1 | 356.8 | 335.7 | 232.2 | 392.7 | 263.8 |
| | 1407.4 | 354.4 | 849.7 | 354.3 | 274.1 | 138.7 | 222.4 | 124.5 | 29.9 | 349.0 | 244.8 |
| | 596.7 | 415.6 | 367.1 | 288.2 | 468.6 | 352.2 | 310.4 | 227.7 | 95.0 | 140.7 | 200.5 |
| | 591.4 | 291.2 | 344.8 | 258.5 | 319.6 | 428.2 | 197.9 | 323.1 | 50.8 | 177.0 | 208.4 |
| | 722.9 | 372.2 | 802.2 | 352.8 | 151.2 | 363.5 | 972.2 | 85.2 | 131.3 | 140.0 | 196.2 |
| | 660.7 | 354.5 | 382.6 | 275.7 | 203.1 | 350.4 | 449.1 | 217.1 | 323.9 | 250.2 | 211.8 |
| | 1084.2 | 460.8 | 409.5 | 450.0 | 400.1 | 431.3 | 329.2 | 194.2 | 275.5 | 198.4 | 167.2 |
| | 856.5 | 1251.7 | 459.9 | 397.8 | 446.1 | 437.1 | 324.7 | 222.5 | 160.9 | 223.4 | 100.7 |
| | 806.3 | 469.1 | 418.6 | 342.6 | 370.9 | 521.8 | 442.5 | 219.1 | 149.0 | 133.7 | 138.3 |
| | 1860.8 | 708.9 | 465.3 | 230.9 | 444.1 | 851.1 | 255.6 | 120.6 | 270.1 | 165.1 | 233.8 |
| | 818.6 | | | | | | | | | | |
| | 1250.8 | | | | | | | | | | |
| | 682.8 | | | | | | | | | | |
| | 815.7 | | | | | | | | | | |
| | 574.9 | | | | | | | | | | |
| | 576.4 | | | | | | | | | | |
| | 1034.9 | | | | | | | | | | |
| | 612.3 | | | | | | | | | | |
| | 829.1 | | | | | | | | | | |
| | 835.0 | | | | | | | | | | |
| | 511.6 | | | | | | | | | | |
| | 503.7 | | | | | | | | | | |
| | 613.1 | | | | | | | | | | |
| | 686.0 | | | | | | | | | | |
| | 305.0 | | | | | | | | | | |
| | 1004.2 | | | | | | | | | | |
| | 708.0 | | | | | | | | | | |
| | 967.9 | | | | | | | | | | |
| | 600.8 | | | | | | | | | | |
| | 724.3 | | | | | | | | | | |
| Ave.: | 803.2 | 545.6 | 479.5 | 319.0 | 326.0 | 448.0 | 386.1 | 207.0 | 171.8 | 217.0 | 196.5 |

TABLE 44
Individual Fractional Inhibition of Tumor Volume

| | | | | | | | | hCBE | hCBE | hCBE |
|---|---|---|---|---|---|---|---|---|---|---|
| | Gem | Gem | | | | | hCBE 50 + Gem | 50 + Gem | 100 + Gem | 100 + Gem |
| | 6.25 | 12.5 | Gem 25 | Gem 50 | hCBE 50 | hCBE 100 | 12.5 | 25 | 12.5 | 25 |
| | 0.032 | 0.632 | 0.703 | 0.774 | 0.245 | 0.556 | 0.582 | 0.711 | 0.511 | 0.672 |
| | 0.559 | −0.058 | 0.559 | 0.659 | 0.827 | 0.723 | 0.845 | 0.963 | 0.566 | 0.695 |
| | 0.483 | 0.543 | 0.641 | 0.417 | 0.562 | 0.614 | 0.717 | 0.882 | 0.825 | 0.750 |
| | 0.637 | 0.571 | 0.678 | 0.602 | 0.467 | 0.754 | 0.598 | 0.937 | 0.780 | 0.741 |
| | 0.537 | 0.001 | 0.561 | 0.812 | 0.547 | −0.210 | 0.894 | 0.837 | 0.826 | 0.756 |
| | 0.559 | 0.524 | 0.657 | 0.747 | 0.564 | 0.441 | 0.730 | 0.597 | 0.688 | 0.736 |
| | 0.426 | 0.490 | 0.440 | 0.502 | 0.463 | 0.590 | 0.758 | 0.657 | 0.753 | 0.792 |
| | −0.558 | 0.427 | 0.505 | 0.445 | 0.456 | 0.596 | 0.723 | 0.800 | 0.722 | 0.875 |
| | 0.416 | 0.479 | 0.574 | 0.538 | 0.350 | 0.449 | 0.727 | 0.815 | 0.834 | 0.828 |
| | 0.117 | 0.421 | 0.712 | 0.447 | −0.060 | 0.682 | 0.850 | 0.664 | 0.794 | 0.709 |
| Ave.: | 0.321 | 0.403 | 0.603 | 0.594 | 0.442 | 0.519 | 0.742 | 0.786 | 0.730 | 0.755 |
| Additive: | | | | | | | 0.845 | 1.045 | 0.922 | 1.122 |
| Difference: | | | | | | | −0.103 | −0.259 | −0.193 | −0.367 |
| | Two-Tailed One-Sample T-Test | | | | | | | | | |
| T-Value: | | | | | | | −3.186 | −6.563 | −5.425 | −18.783 |
| DF: | | | | | | | 9 | 9 | 9 | 9 |
| P-Value: | | | | | | | 0.0111 | 0.0001 | 0.0004 | <0.0001 |

In sum, the combination treatment of the LTβ receptor-activating mAb huCBE11 and the chemotherapeutic agent gemcitabine in athymic nude mice implanted subcutaneously with WiDr human colorectal adenocarcinoma showed an effect of combination treatment with huCBE11 and gemcitabine that was determined to be synergistic at low concentrations of huCBE11 and gemcitabine.

B. Antitumor Efficacy of Combination of huCBE11 with Gemcitabine Using KM-20L2 Mouse Model

An additional human colorectal adenocarcinoma mouse model system, the KM-20L2 model, was also utilized to determine whether administration of a nucleoside analog chemotherapeutic agent, e.g., gemcitabine, in combination with huCBE11 has supra-additive, e.g., synergistic or potentiating, antitumor activity.

Dose ranging studies were initially performed to determine the appropriate gemcitabine and huCBE11 dose(s) for studying the combined antitumor effects of gemcitabine and huCBE11. Increasing doses of gemcitabine from 25 mg/kg to 140 mg/kg were administered to athymic nude mice implanted subcutaneously with KM-20L2 tumor cells (day 0). Tumor take rate was 100% on implantation, and 110 mice within a tight size range were selected to initiate treatments. Significant inhibition of tumor growth was observed from Day 10 to the last day of study, Day 41, in the gemcitabine 140 mg/kg (P<0.05 Day 10; P<0.001 Days 14-41), 100 mg/kg (P<0.05 Day 10; P<0.001 Days 14-41), 50 mg/kg (P<0.001 Days 10-41), and 25 mg/kg (P<0.01 Days 19 and 41; P<0.001 Days 14-37) groups compared with the saline control group. The % T/C was at or below 42% on Days 14 or 17 and remained there for the duration of the study in the gemcitabine 140 mg/kg, 100 mg/kg, and 50 mg/kg groups. The % T/C was at or below 42% on Day 17 in the gemcitabine 25 mg/kg group and remained there through Day 31. In a separate study, 5, 10 and 20 mg/kg doses of gemcitabine were examined for antitumor activity in the KM-20L2 human adenocarcinoma xenograft model. Tumor take rate was 100% on implantation, and 129 mice within a tight size range were selected to initiate treatments. Tumor growth in the vehicle control group was well within the typical range seen in this laboratory with this model. Significant inhibition of tumor growth was observed on Days 13-55 in the gemcitabine 20 mg/kg (P<0.001 Days 13-47, P<0.01 Days 50-55), Days 16-55 in the 10 mg/kg (P<0.01 Days 16-50, P<0.05 Day 55), and Days 13-43 in the 5 mg/kg (P<0.01 Days 20-23, P<0.05 Days 13-16 and Days 27-43) groups compared with the vehicle control group. The % T/C was at or below 42% on Day 16 and remained there through Day 34 in the gemcitabine 20 mg/kg group. Thus, gemcitabine was determined to be active against the KM-20L2 tumor model based on the NCI criteria of activity (% T/C of 42 or less).

In a parallel dose ranging study, the activity of huCBE11 in the KM-20L2 human adenocarcinoma xenograft model was examined. huCBE11 was administered at 0.2, 2, 4, and 20 mg/kg. Tumor take rate was 99.5% on implantation, and 110 mice within a tight size range were selected to initiate treatments. Tumor growth was significantly (P<0.05) decreased in the huCBE11 2 mg/kg (Days 28-33) and 4 mg/kg dose (Days 21-28) groups compared with the vehicle control group. The lowest % T/C observed in these dose groups was >42%. In a separate study, 0.2, 2, and 4 mg/kg doses of gemcitabine were administered to KM-20L2 model mice. For these mice, tumor growth was significantly decreased on Days 20-55 in the huCBE11 4 mg/kg (P<0.01 Day 20-23; P<0.001 Days 27-55) and 2 mg/kg (P<0.01 Days 20-23, P<0.001 Days 27-55) groups compared with the vehicle control group. The % T/C was at or below 42% on Days 50 and 55 in the hCBE11 4 mg/kg group and on Days 41 to 55 in the hCBE11 2 mg/kg group. Thus, huCBE11 was determined to be active against the KM-20L2 tumor model based on the NCI criteria of activity (% T/C of 42 or less).

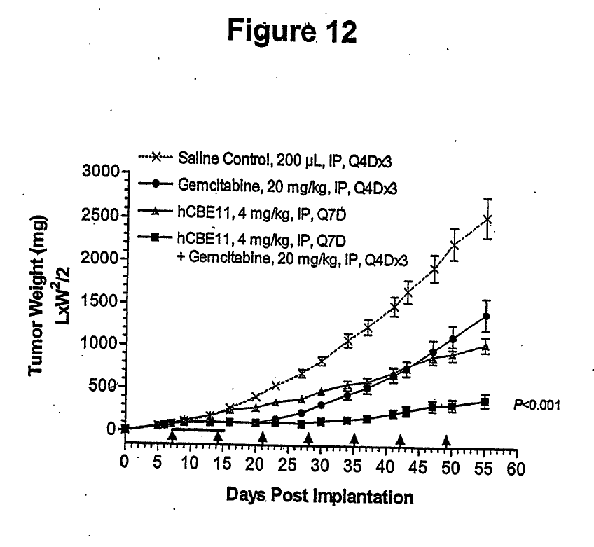

The combination effect of huCBE11 and gemcitabine was also examined. Cohorts of animals were treated: with saline control (0.9% sterile saline), with decreasing doses of gemcitabine (20, 10 and 5 mg/kg), with decreasing doses of huCBE11 (4, 2 and 0.2 mg/kg), or with combinations of doses of huCBE11 plus gemcitabine (4 mg/kg huCBE11+20 mg/kg gemcitabine, 4 mg/kg huCBE11+10 mg/kg gemcitabine, 0.2 mg/kg huCBE11+20 mg/kg gemcitabine, 0.2 mg/kg huCBE11+10 mg/kg gemcitabine, 4 mg/kg huCBE11+5 mg/kg gemcitabine, 8 mg/kg huCBE11+10 mg/kg gemcitabine, and 20 mg/kg huCBE11+25 mg/kg gemcitabine) using the same regimens as the single agents beginning on Day 7. All treatments began when the tumors reached an average of 5 millimeters (mm) in length by 5 mm in width. The combination treatment of huCBE11 4 mg/kg and gemcitabine 20 mg/kg showed significant inhibition of tumor growth compared with huCBE11 4 mg/kg alone on Days 10 through 55 (P<0.01 Day 10; P<0.001 Days 13-55) (FIG. 12). The % T/C in this dose group was at or below 42% from Days 16 to 55, with a low of 13.6% on Day 37. The combination treatment of hCBE11 4 mg/kg and gemcitabine 10 mg/kg showed significant inhibition of tumor growth compared with hCBE11 4 mg/kg alone on Days 13-55 (P<0.001 Days 16-50, P<0.01 Days 13 and 55). The % T/C in this dose group was at or below 42% on Days 20 through 55, with a low of 15.8%. The combination treatment of hCBE11 0.2 mg/kg and gemcitabine 20 mg/kg showed significant inhibition of tumor growth compared with gemcitabine 20 mg/kg alone on Days 27-55 (P<0.05 Days 27, 37, and 43; P<0.01 Days 30-34, 41, 47-55). The % T/C was at or below 42% in this group from Days 16 through 55, with a low of 19.8% on Day 30. The combination treatment of hCBE11 0.2 mg/kg and gemcitabine 10 mg/kg did not show significant inhibition of tumor growth compared with gemcitabine 10 mg/kg alone and the % T/C was not at or below 42% at anytime during the study. The combination treatment of hCBE11 4 mg/kg and gemcitabine 5 mg/kg showed significant inhibition of tumor growth compared with hCBE11 4 mg/kg alone on Days 9-43 and on Day 55 (P<0.05 Days 9, 41, 43, and 55; P<0.01 Days 13, 20, 27, 34, and 37; P<0.001 Days 16, 23, and 30. The % T/C was at or below 42% in this dose group on Days 20 through 55, with a low of 25.6% on Day 37. While it was not possible to compare the inhibition of tumor growth in the hCBE11 8 mg/kg plus gemcitabine 10 mg/kg group with hCBE11 8 mg/kg or in the hCBE11 20 mg/kg plus gemcitabine 25 mg/kg group with hCBE11 20 mg/kg, the % T/C observed in these groups was at or below 42% on Days 16 through 55. In sum, the above six combination treatments of huCBE11 plus gemcitabine were determined to be active in the KM-20L2 tumor model based on the NCI criteria of activity (% T/C of 42 or less). Each of these six huCBE11+gemcitabine combination therapies produced statistically significant decreases in tumor growth.

Using the tumor weight data obtained from these combination treatment studies, statistical comparisons were performed to determine whether the combination of huCBE11 plus gemcitabine was synergistic in its mode of action. Because treatment of KM-20L2 tumor-bearing mice with gemcitabine or huCBE11 alone produced dose-responsive antitumor efficacy, synergism of the huCBE11+gemcitabine combination could be formally assessed by calculating the Combination Index (CI) (Chou, 1984).

To enable assessment of whether supra-additive effects occur with combination administration of huCBE11 and gemcitabine, antitumor efficacy was first determined by comparing each treatment group's tumor volume with the control group's tumor volume. Mean tumor volume decrease was calculated as the difference between the control group and the treatment group in mean tumor volume. The fractional inhibition of tumor volume, i.e., the fraction affected (Fa), was calculated by dividing the treatment group mean tumor volume decrease by the control group mean tumor volume. An Fa of 1.000 indicated complete inhibition of the tumor. Those doses used to assess synergistic drug action in the current study were given in a fixed ratio of 4:5 (mg/kg gemcitabine:mg/kg huCBE11). This ratio was based on the ratio of the median effect doses for the 2 agents. This ratio was based on the ratio of the median effect doses for the 2 agents determined in previous pilot studies. Table 28 shows the dose-effect relationships for separate and combination treatments.

TABLE 28
huCBE11 and Gemcitabine (Gem): Mean Tumor Size and Calculated Fractional Inhibition by
Treatment and Day for Separate and Combination Treatments of Gemcitabine and huCBE11

| | Dose | | Dose | Day | | | | | |
|---|---|---|---|---|---|---|---|---|---|
| Treatment | (mg/kg) | Cotreatment | (mg/kg) | 9 | 13 | 16 | 20 | 23 | 27 |

Mean Tumor Volume (cubic mm)

| | | | | | | | | | |
|---|---|---|---|---|---|---|---|---|---|
| Vehicle | | | | 111.8 | 172.6 | 265.0 | 401.0 | 527.2 | 668.6 |
| Gem | 5 | | | 101.0 | 141.8 | 204.1 | 276.8 | 371.6 | 506.7 |
| Gem | 10 | | | 102.4 | 140.9 | 169.0 | 231.7 | 311.2 | 417.4 |
| Gem | 20 | | | 105.9 | 111.0 | 99.8 | 101.1 | 147.3 | 225.1 |
| huCBE11 | 0.2 | | | 104.8 | 160.4 | 250.5 | 371.9 | 518.7 | 673.8 |
| huCBE11 | 2 | | | 100.3 | 162.7 | 233.5 | 263.7 | 339.2 | 388.7 |
| huCBE11 | 4 | | | 125.8 | 180.9 | 247.6 | 278.0 | 344.3 | 385.8 |
| huCBE11 | 0.2 | Gem | 10 | 97.2 | 113.6 | 143.0 | 174.5 | 241.5 | 348.4 |
| huCBE11 | 4 | Gem | 10 | 106.5 | 125.6 | 114.8 | 106.6 | 113.3 | 122.0 |
| huCBE11 | 0.2 | Gem | 20 | 110.8 | 110.7 | 107.4 | 101.7 | 109.6 | 138.1 |
| huCBE11 | 4 | Gem | 20 | 97.7 | 102.2 | 103.2 | 101.5 | 106.4 | 99.4 |
| huCBE11 | 4 | Gem | 5 | 103.7 | 130.7 | 137.4 | 155.0 | 160.9 | 191.7 |
| huCBE11 | 8 | Gem | 10 | 94.8 | 103.6 | 98.4 | 82.0 | 87.9 | 98.7 |
| huCBE11 | 20 | Gem | 25 | 99.5 | 110.9 | 93.3 | 77.0 | 83.5 | 85.1 |

Mean Tumor Volume Decrease (cubic mm)

| | | | | | | | | | |
|---|---|---|---|---|---|---|---|---|---|
| Vehicle | | | | 0.0 | 0.0 | 0.0 | 0.0 | 0.0 | 0.0 |
| Gem | 5 | | | 10.8 | 30.8 | 60.9 | 124.2 | 155.6 | 161.9 |
| Gem | 10 | | | 9.4 | 31.7 | 96.0 | 169.3 | 216.0 | 251.2 |
| Gem | 20 | | | 5.9 | 61.6 | 165.2 | 299.9 | 379.9 | 443.5 |
| huCBE11 | 0.2 | | | 7.0 | 12.2 | 14.5 | 29.1 | 8.5 | −5.2 |
| huCBE11 | 2 | | | 11.5 | 9.9 | 31.5 | 137.3 | 188.0 | 279.9 |
| huCBE11 | 4 | | | −14.0 | −8.3 | 17.4 | 123.0 | 182.9 | 282.8 |
| huCBE11 | 0.2 | Gem | 10 | 14.6 | 59.0 | 122.0 | 226.5 | 285.7 | 320.2 |
| huCBE11 | 4 | Gem | 10 | 5.3 | 47.0 | 150.2 | 294.4 | 413.9 | 546.6 |
| huCBE11 | 0.2 | Gem | 20 | 1.0 | 61.9 | 157.6 | 299.3 | 417.6 | 530.5 |
| huCBE11 | 4 | Gem | 20 | 14.1 | 70.4 | 161.8 | 299.5 | 420.8 | 569.2 |
| huCBE11 | 4 | Gem | 5 | 8.1 | 41.9 | 127.6 | 246.0 | 366.3 | 476.9 |
| huCBE11 | 8 | Gem | 10 | 17.0 | 69.0 | 166.6 | 319.0 | 439.3 | 569.9 |
| huCBE11 | 20 | Gem | 25 | 12.3 | 61.7 | 171.7 | 324.0 | 443.7 | 583.5 |

Mean Fraction Affected (Fa)

| | | | | | | | | | |
|---|---|---|---|---|---|---|---|---|---|
| Vehicle | | | | 0.000 | 0.000 | 0.000 | 0.000 | 0.000 | 0.000 |
| Gem | 5 | | | 0.097 | 0.178 | 0.230 | 0.310 | 0.295 | 0.242 |
| Gem | 10 | | | 0.084 | 0.184 | 0.362 | 0.422 | 0.410 | 0.376 |
| Gem | 20 | | | 0.053 | 0.357 | 0.623 | 0.748 | 0.721 | 0.663 |
| huCBE11 | 0.2 | | | 0.063 | 0.071 | 0.055 | 0.073 | 0.016 | −0.008 |
| huCBE11 | 2 | | | 0.103 | 0.057 | 0.119 | 0.342 | 0.357 | 0.419 |
| huCBE11 | 4 | | | −0.125 | −0.048 | 0.066 | 0.307 | 0.347 | 0.423 |
| huCBE11 | 0.2 | Gem | 10 | 0.131 | 0.342 | 0.460 | 0.565 | 0.542 | 0.479 |
| huCBE11 | 4 | Gem | 10 | 0.047 | 0.272 | 0.567 | 0.734 | 0.785 | 0.818 |
| huCBE11 | 0.2 | Gem | 20 | 0.009 | 0.359 | 0.595 | 0.746 | 0.792 | 0.793 |
| huCBE11 | 4 | Gem | 20 | 0.126 | 0.408 | 0.611 | 0.747 | 0.798 | 0.851 |
| huCBE11 | 4 | Gem | 5 | 0.072 | 0.243 | 0.482 | 0.613 | 0.695 | 0.713 |
| huCBE11 | 8 | Gem | 10 | 0.152 | 0.400 | 0.629 | 0.796 | 0.833 | 0.852 |
| huCBE11 | 20 | Gem | 25 | 0.110 | 0.357 | 0.648 | 0.808 | 0.842 | 0.873 |

Mean Fraction Affected/Fraction Unaffected (Fa/Fu)

| | | | | | | | | | |
|---|---|---|---|---|---|---|---|---|---|
| Vehicle | | | | 0.000 | 0.000 | 0.000 | 0.000 | 0.000 | 0.000 |
| Gem | 5 | | | 0.107 | 0.217 | 0.298 | 0.449 | 0.419 | 0.320 |
| Gem | 10 | | | 0.092 | 0.225 | 0.568 | 0.731 | 0.694 | 0.602 |
| Gem | 20 | | | 0.056 | 0.555 | 1.655 | 2.966 | 2.579 | 1.970 |
| huCBE11 | 0.2 | | | 0.067 | 0.076 | 0.058 | 0.078 | 0.016 | −0.008 |
| huCBE11 | 2 | | | 0.115 | 0.061 | 0.135 | 0.521 | 0.554 | 0.720 |
| huCBE11 | 4 | | | −0.111 | −0.046 | 0.070 | 0.442 | 0.531 | 0.733 |
| huCBE11 | 0.2 | Gem | 10 | 0.150 | 0.519 | 0.853 | 1.298 | 1.183 | 0.919 |
| huCBE11 | 4 | Gem | 10 | 0.050 | 0.374 | 1.308 | 2.762 | 3.653 | 4.480 |
| huCBE11 | 0.2 | Gem | 20 | 0.009 | 0.559 | 1.467 | 2.943 | 3.810 | 3.841 |
| huCBE11 | 4 | Gem | 20 | 0.144 | 0.689 | 1.568 | 2.951 | 3.955 | 5.726 |
| huCBE11 | 4 | Gem | 5 | 0.078 | 0.321 | 0.929 | 1.587 | 2.277 | 2.488 |
| huCBE11 | 8 | Gem | 10 | 0.179 | 0.666 | 1.693 | 3.890 | 4.998 | 5.774 |
| huCBE11 | 20 | Gem | 25 | 0.124 | 0.556 | 1.840 | 4.208 | 5.314 | 6.857 |

| | Day | | | | |
|---|---|---|---|---|---|
| Treatment | 30 | 34 | 37 | 41 | 43 |
| Mean Tumor Volume (cubic mm) | | | | | |
| Vehicle | 816.2 | 1050.3 | 1208.0 | 1439.4 | 1606.7 |
| Gem | 616.2 | 740.4 | 877.5 | 1075.3 | 1244.3 |
| Gem | 499.5 | 642.3 | 723.7 | 865.2 | 975.7 |
| Gem | 322.6 | 443.0 | 522.1 | 674.3 | 753.0 |
| huCBE11 | 792.1 | 926.4 | 1048.5 | 1266.0 | 1452.0 |
| huCBE11 | 450.1 | 519.9 | 580.1 | 623.2 | 662.7 |
| huCBE11 | 480.1 | 558.9 | 591.1 | 702.0 | 775.1 |
| huCBE11 | 456.2 | 574.9 | 689.4 | 877.3 | 1012.8 |
| huCBE11 | 135.6 | 171.1 | 204.3 | 272.4 | 327.8 |
| huCBE11 | 164.5 | 221.7 | 272.9 | 337.6 | 395.0 |
| huCBE11 | 127.0 | 147.4 | 168.5 | 227.3 | 260.5 |
| huCBE11 | 216.2 | 279.0 | 318.0 | 405.3 | 469.8 |
| huCBE11 | 125.3 | 155.0 | 199.7 | 249.1 | 299.7 |
| huCBE11 | 96.7 | 124.2 | 165.4 | 204.8 | 264.0 |
| Mean Tumor Volume Decrease (cubic mm) | | | | | |
| Vehicle | 0.0 | 0.0 | 0.0 | 0.0 | 0.0 |
| Gem | 200.0 | 309.9 | 330.5 | 364.1 | 362.4 |
| Gem | 316.7 | 408.0 | 484.3 | 574.2 | 631.0 |
| Gem | 493.6 | 607.3 | 685.9 | 765.1 | 853.7 |
| huCBE11 | 24.1 | 123.9 | 159.5 | 173.4 | 154.7 |
| huCBE11 | 366.1 | 530.4 | 627.9 | 816.2 | 944.0 |
| huCBE11 | 336.1 | 491.4 | 616.9 | 737.4 | 831.6 |
| huCBE11 | 360.0 | 475.4 | 518.6 | 562.1 | 593.9 |
| huCBE11 | 680.6 | 879.2 | 1003.7 | 1167.0 | 1278.9 |
| huCBE11 | 651.7 | 828.6 | 935.1 | 1101.8 | 1211.7 |
| huCBE11 | 689.2 | 902.9 | 1039.5 | 1212.1 | 1346.2 |
| huCBE11 | 600.0 | 771.3 | 890.0 | 1034.1 | 1136.9 |
| huCBE11 | 690.9 | 895.3 | 1008.3 | 1190.3 | 1307.0 |
| huCBE11 | 719.5 | 926.1 | 1042.6 | 1234.6 | 1342.7 |
| Mean Fraction Affected (Fa) | | | | | |
| Vehicle | 0.000 | 0.000 | 0.000 | 0.000 | 0.000 |
| Gem | 0.245 | 0.295 | 0.274 | 0.253 | 0.226 |
| Gem | 0.388 | 0.388 | 0.401 | 0.399 | 0.393 |
| Gem | 0.605 | 0.578 | 0.568 | 0.532 | 0.531 |
| huCBE11 | 0.030 | 0.118 | 0.132 | 0.120 | 0.096 |
| huCBE11 | 0.449 | 0.505 | 0.520 | 0.567 | 0.588 |
| huCBE11 | 0.412 | 0.468 | 0.511 | 0.512 | 0.518 |
| huCBE11 | 0.441 | 0.453 | 0.429 | 0.391 | 0.370 |
| huCBE11 | 0.834 | 0.837 | 0.831 | 0.811 | 0.796 |
| huCBE11 | 0.798 | 0.789 | 0.774 | 0.765 | 0.754 |
| huCBE11 | 0.844 | 0.860 | 0.861 | 0.842 | 0.838 |
| huCBE11 | 0.735 | 0.734 | 0.737 | 0.718 | 0.708 |
| huCBE11 | 0.846 | 0.852 | 0.835 | 0.827 | 0.813 |
| huCBE11 | 0.882 | 0.882 | 0.863 | 0.858 | 0.836 |
| Mean Fraction Affected/Fraction Unaffected (Fa/Fu) | | | | | |
| Vehicle | 0.000 | 0.000 | 0.000 | 0.000 | 0.000 |
| Gem | 0.325 | 0.419 | 0.377 | 0.339 | 0.291 |
| Gem | 0.634 | 0.635 | 0.669 | 0.664 | 0.647 |
| Gem | 1.530 | 1.371 | 1.314 | 1.135 | 1.134 |
| huCBE11 | 0.030 | 0.134 | 0.152 | 0.137 | 0.107 |
| huCBE11 | 0.813 | 1.020 | 1.082 | 1.310 | 1.424 |
| huCBE11 | 0.700 | 0.879 | 1.044 | 1.050 | 1.073 |
| huCBE11 | 0.789 | 0.827 | 0.752 | 0.641 | 0.586 |
| huCBE11 | 5.019 | 5.139 | 4.913 | 4.284 | 3.901 |
| huCBE11 | 3.962 | 3.737 | 3.427 | 3.264 | 3.068 |
| huCBE11 | 5.427 | 6.126 | 6.169 | 5.333 | 5.168 |
| huCBE11 | 2.775 | 2.765 | 2.799 | 2.551 | 2.420 |
| huCBE11 | 5.514 | 5.776 | 5.049 | 4.778 | 4.361 |
| huCBE11 | 7.441 | 7.457 | 6.304 | 6.028 | 5.086 |

The data for fixed ratio combination huCBE11+gemcitabine therapy presented in Table 28 allowed for formal assessment of synergism to be performed through calculation of the CI using CalcuSyn V1.1 (Biosoft, Cambridge, UK) software for Windows-based dose-effect analysis. For treatments given in combination, a CI equal to 1 indicated additive efficacy. CI less than 1 indicated synergism. CI greater than 1 indicated antagonism. Dose-effect relationships used in CI calculations are shown in Table 29.

TABLE 29
Dose-Effect Relationships of huCBE11 and Gemcitabine for Synergism Calculations

| | | Day | | | | | | | | |
|---|---|---|---|---|---|---|---|---|---|---|
| Dose | Dose | 16 | 20 | 23 | 27 | 30 | 34 | 37 | 41 | 43 |

| | | | | |
|---|---|---|---|---|
| Treatment | (mg/kg) | Cotreatment | (mg/kg) | Fraction affected |

Given Separately

| | | | | | | | | | | |
|---|---|---|---|---|---|---|---|---|---|---|
| huCBE11 | 0.2 | 0.055 | 0.073 | 0.016 | 0.000 | 0.030 | 0.118 | 0.132 | 0.120 | 0.096 |
| | 2.0 | 0.119 | 0.342 | 0.357 | 0.419 | 0.449 | 0.505 | 0.520 | 0.567 | 0.588 |
| | 4.0 | 0.120 | 0.307 | 0.347 | 0.423 | 0.412 | 0.468 | 0.511 | 0.512 | 0.518 |
| Gem | 5 | 0.230 | 0.310 | 0.295 | 0.242 | 0.245 | 0.295 | 0.274 | 0.253 | 0.226 |
| | 10 | 0.362 | 0.422 | 0.410 | 0.376 | 0.388 | 0.388 | 0.401 | 0.399 | 0.393 |
| | 20 | 0.623 | 0.748 | 0.721 | 0.663 | 0.605 | 0.578 | 0.568 | 0.532 | 0.531 |

Combined (4:5)

| | | | | | | | | | | | | |
|---|---|---|---|---|---|---|---|---|---|---|---|---|
| huCBE11 | 4 | Gem | 5 | 0.482 | 0.613 | 0.695 | 0.713 | 0.735 | 0.734 | 0.737 | 0.718 | 0.708 |
| | 8 | | 10 | 0.629 | 0.796 | 0.833 | 0.852 | 0.846 | 0.852 | 0.835 | 0.827 | 0.813 |
| | 20 | | 25 | 0.648 | 0.808 | 0.842 | 0.873 | 0.882 | 0.882 | 0.863 | 0.858 | 0.836 |

Median effect doses for separate and combination treatments of huCBE11 and gemcitabine are shown in Table 30. Potency and shape of the dose-response relation for separate and combination treatments of gemcitabine and huCBE11 are shown in Tables 31 and 32, respectively. The CI calculated for the exact levels of the experimental doses used in this study are given in Table 32.

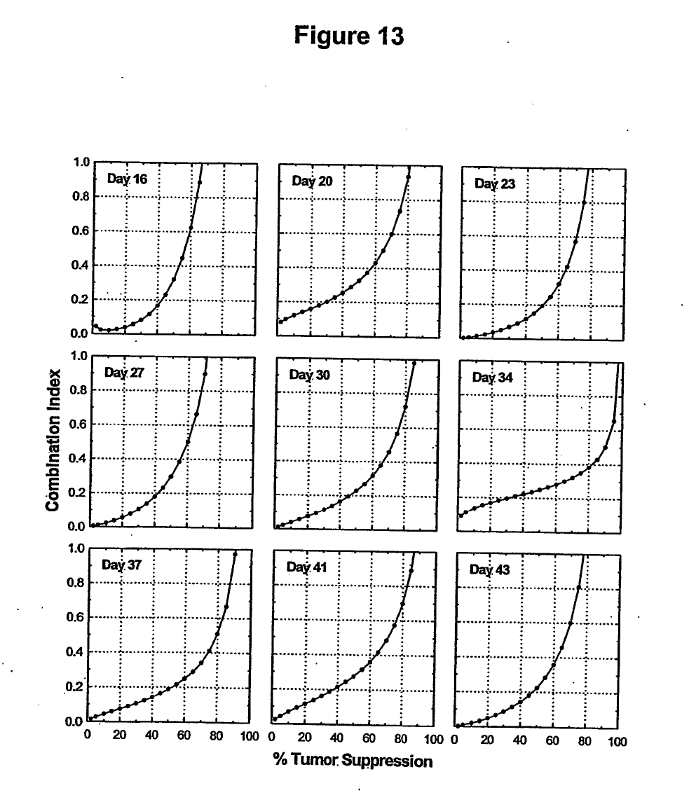

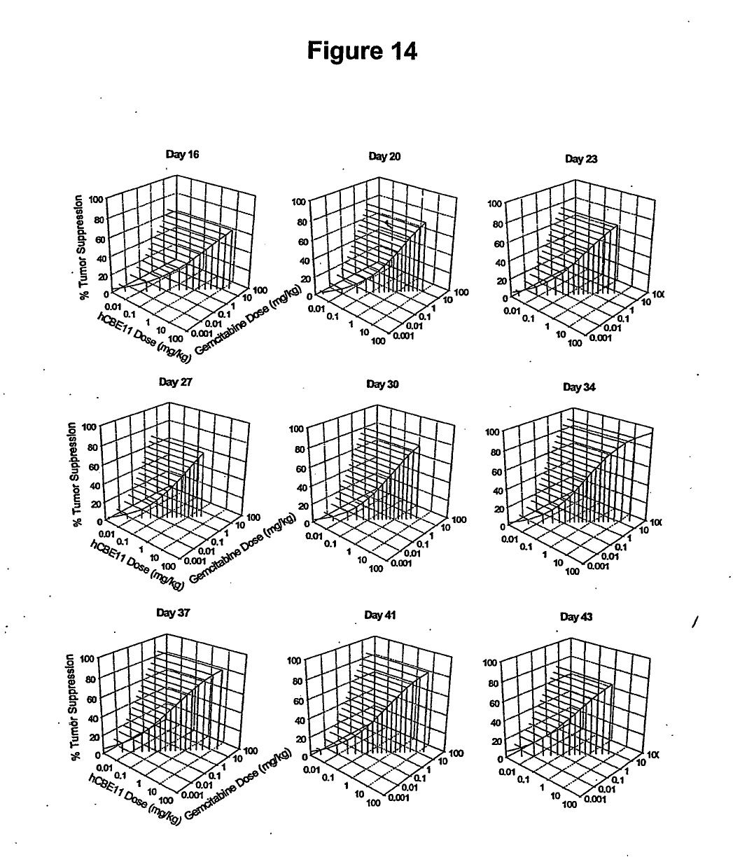

Because the current study employed drugs that are thought to have entirely independent modes of action, mutually nonexclusive CI values probably apply (See Tables 3 and 32-34). Combination doses using 4 mg/kg huCBE11+5 mg/kg gemcitabine, 8 mg/kg huCBE11+10 mg/kg gemcitabine, and 20 mg/kg huCBE11+25 mg/kg gemcitabine (i.e., fixed-ratio combinations of 4:5) showed a synergistic effect on Days 34-37. The lower of these 2 dose combinations showed synergism on most treatment days (Table 32). Simulations of the CI over a range of dose levels for the combination are given in Tables 33 and 34 and overall interpretation of the degree of synergism or antagonism indicated by the CI is given in Table 3. Synergistic effects were present throughout the entire treatment period. CI as a function of percent tumor suppression is shown in FIG. 13. Synergism was most marked at levels of tumor suppression below 60%. Peak synergistic effects for the combination were observed on Day 34. The dose range over which synergistic effects on efficacy occurred is shown in FIG. 14. The dose range that produced 20% to 80% tumor suppression combined the 2 drugs at dose levels between 0.1 and 10 mg/kg (FIG. 14). The results of this analysis demonstrated that fixed ratio combination treatment (4:5) using huCBE11+gemcitabine showed synergistic antitumor efficacy.

TABLE 30
Determination of Synergism: Median Effect Doses (mg/kg; Potency)
for Synergism of huCBE11 and Gemcitabine

| | | | Gemcitabine | |
|---|---|---|---|---|
| | huCBE11 | | Given | |
| | Given Separately | Combined (4:5) | Separately | Combined (4:5) |
| Day | Median Effect Dose (95% Confidence Interval) | | | |
| 16 | 2077 | 3.6 | 14.1 | 4.5 |
| | (83-51942) | (1.1-11.4) | (11.7-17.0) | (1.4-14.2) |
| 20 | 9.5 | 1.3 | 10.1 | 1.7 |
| | (2.2-42.0) | (0.1-13.2) | (7.4-13.7) | (0.2-16.5) |
| 23 | 4.8 | 0.6 | 10.8 | 0.7 |
| | (2.0-11.7) | (0.1-4.2) | (8.1-14.3) | (0.1-5.3) |
| 27 | 3.3 | 0.7 | 12.8 | 0.9 |
| | (1.6-6.7) | (0.1-4.1) | (10.4-15.8) | (0.1-5.1) |
| 30 | 3.8 | 0.6 | 14.1 | 0.8 |
| | (1.5-9.6) | (0.2-1.8) | (12.7-15.6) | (0.3-2.3) |
| 34 | 3.2 | 0.6 | 14.8 | 0.8 |
| | (1.3-8.4) | (0.2-2.2) | (11.7-18.7) | (0.2-2.7) |
| 37 | 2.7 | 0.4 | 15.0 | 0.5 |
| | (1.3-5.6) | (0.2-1.1) | (14.1-16.0) | (0.2-1.3) |
| 41 | 2.4 | 0.6 | 16.8 | 0.7 |
| | (1.0-6.2) | (0.2-1.8) | (15.3-18.5) | (0.2-2.2) |
| 43 | 2.4 | 0.4 | 16.9 | 0.6 |
| | (0.9-6.1) | (0.1-1.5) | (14.5-19.7) | (0.2-1.9) |

TABLE 31
Determination of Synergism: Dose-Effect Curve Characteristics for Separate and
Combination huCBE11 and Gemcitabine Treatments

| | | huCBE11 | | | Gemcitabine | | | huCBE11 + Gemcitabine | | |
|---|---|---|---|---|---|---|---|---|---|---|
| Day | Value | Slope | Y-Intercept | R | Slope | Y-Intercept | R | Slope | Y-Intercept | R |
| 16 | Mean | 0.30 | −1.01 | 0.9774 | 1.23 | −1.42 | 0.9898 | 0.41 | −0.23 | 0.8811 |
| | SEM | 0.07 | 0.04 | | 0.18 | 0.18 | | 0.22 | 0.21 | |
| 20 | Mean | 0.64 | −0.62 | 0.9556 | 1.36 | −1.37 | 0.9629 | 0.58 | −0.07 | 0.8608 |
| | SEM | 0.20 | 0.11 | | 0.38 | 0.39 | | 0.34 | 0.34 | |
| 23 | Mean | 1.26 | −0.86 | 0.9728 | 1.31 | −1.35 | 0.9688 | 0.50 | 0.12 | 0.8611 |
| | SEM | 0.30 | 0.17 | | 0.34 | 0.35 | | 0.30 | 0.29 | |
| 27 | Mean | 3.19 | −1.64 | 0.9756 | 1.31 | −1.45 | 0.9852 | 0.61 | 0.09 | 0.9049 |
| | SEM | 0.72 | 0.40 | | 0.23 | 0.24 | | 0.29 | 0.28 | |
| 30 | Mean | 1.14 | −0.66 | 0.9654 | 1.12 | −1.29 | 0.9969 | 0.60 | 0.12 | 0.9568 |
| | SEM | 0.31 | 0.17 | | 0.09 | 0.09 | | 0.18 | 0.18 | |
| 34 | Mean | 0.69 | −0.35 | 0.9587 | 0.86 | −1.00 | 0.9854 | 0.60 | 0.13 | 0.9401 |
| | SEM | 0.21 | 0.12 | | 0.15 | 0.15 | | 0.22 | 0.21 | |
| 37 | Mean | 0.70 | −0.30 | 0.9716 | 0.90 | −1.06 | 0.9989 | 0.49 | 0.19 | 0.9430 |
| | SEM | 0.17 | 0.09 | | 0.04 | 0.04 | | 0.17 | 0.17 | |
| 41 | Mean | 0.76 | −0.29 | 0.9518 | 0.87 | −1.07 | 0.9979 | 0.52 | 0.13 | 0.9433 |
| | SEM | 0.24 | 0.14 | | 0.06 | 0.06 | | 0.18 | 0.18 | |
| 43 | Mean | 0.86 | −0.32 | 0.9484 | 0.98 | −1.20 | 0.9949 | 0.45 | 0.16 | 0.9216 |
| | SEM | 0.29 | 0.16 | | 0.10 | 0.10 | | 0.19 | 0.18 | |

TABLE 32
Determination of Synergism: Combination Indices (CIs) for Experimental
Values

| | | | | Mechanism of Action | | | |
|---|---|---|---|---|---|---|---|
| | | | Fraction | Mutually | | | |
| | Dose (mg/kg) | | Af- | Exclusive | | Nonexclusive | |
| Day | huCBE11 | Gem | fected | CI | Synergism | CI | Synergism |
| 16 | 4 | 5 | 0.482 | 0.378 | +++ | 0.379 | +++ |
| | 8 | 10 | 0.629 | 0.463 | +++ | 0.463 | +++ |
| | 20 | 25 | 0.648 | 1.082 | ± | 1.084 | ± |
| 20 | 4 | 5 | 0.613 | 0.559 | +++ | 0.631 | +++ |
| | 8 | 10 | 0.796 | 0.465 | +++ | 0.501 | +++ |
| | 20 | 25 | 0.808 | 1.085 | ± | 1.276 | −− |
| 23 | 4 | 5 | 0.695 | 0.679 | +++ | 0.786 | ++ |
| | 8 | 10 | 0.833 | 0.735 | ++ | 0.862 | + |
| | 20 | 25 | 0.842 | 1.746 | −−− | 2.458 | −−− |
| 27 | 4 | 5 | 0.713 | 1.115 | − | 1.295 | −− |
| | 8 | 10 | 0.852 | 1.620 | −−− | 1.911 | −−− |
| | 20 | 25 | 0.873 | 3.795 | −−−− | 5.297 | −−−− |
| 30 | 4 | 5 | 0.735 | 0.569 | +++ | 0.630 | +++ |
| | 8 | 10 | 0.846 | 0.623 | +++ | 0.695 | +++ |
| | 20 | 25 | 0.882 | 1.186 | − | 1.448 | −− |
| 34 | 4 | 5 | 0.734 | 0.387 | +++ | 0.417 | +++ |
| | 8 | 10 | 0.852 | 0.284 | ++++ | 0.301 | +++ |
| | 20 | 25 | 0.882 | 0.498 | +++ | 0.552 | +++ |
| 37 | 4 | 5 | 0.737 | 0.441 | +++ | 0.476 | +++ |
| | 8 | 10 | 0.835 | 0.396 | +++ | 0.428 | +++ |
| | 20 | 25 | 0.863 | 0.738 | ++ | 0.851 | + |
| 41 | 4 | 5 | 0.718 | 0.578 | +++ | 0.626 | +++ |
| | 8 | 10 | 0.827 | 0.513 | +++ | 0.554 | +++ |
| | 20 | 25 | 0.858 | 0.949 | ± | 1.092 | ± |
| 43 | 4 | 5 | 0.708 | 0.726 | ++ | 0.799 | ++ |
| | 8 | 10 | 0.813 | 0.748 | ++ | 0.829 | ++ |
| | 20 | 25 | 0.836 | 1.560 | −−− | 1.918 | −−− |

++++ Strong synergism
+++ Synergism
++ Moderate synergism
+ Slight synergism
± Nearly additive
− Slight antagonism
−− Moderate antagonism
−−− Antagonism
−−−− Strong antagonism

TABLE 33
Determination of Synergism: Simulation of Combination Indices (CIs):
Mutally Exclusive Modes of Action

| | | Dose (mg/kg) | | | | Dose (mg/kg) | | |
|---|---|---|---|---|---|---|---|---|
| Fa | CI | huCBE11 | Gem | Symbol | CI | huCBE11 | Gem | Symbol |
| | Day 16 | | | | Day 20 | | | |
| 0.02 | 0.043 | 0.00025 | 0.00031 | +++++ | 0.079 | 0.00161 | 0.00201 | +++++ |
| 0.05 | 0.022 | 0.0026 | 0.0032 | +++++ | 0.097 | 0.0082 | 0.0103 | +++++ |
| 0.10 | 0.019 | 0.0161 | 0.0202 | +++++ | 0.117 | 0.0299 | 0.0374 | ++++ |
| 0.15 | 0.025 | 0.0504 | 0.0630 | +++++ | 0.135 | 0.0664 | 0.0830 | ++++ |
| 0.20 | 0.038 | 0.119 | 0.148 | +++++ | 0.154 | 0.121 | 0.151 | ++++ |
| 0.25 | 0.056 | 0.241 | 0.301 | +++++ | 0.172 | 0.199 | 0.249 | ++++ |
| 0.30 | 0.082 | 0.447 | 0.559 | +++++ | 0.193 | 0.307 | 0.384 | ++++ |
| 0.35 | 0.118 | 0.784 | 0.980 | ++++ | 0.215 | 0.455 | 0.569 | ++++ |
| 0.40 | 0.166 | 1.33 | 1.66 | ++++ | 0.240 | 0.66 | 0.82 | ++++ |
| 0.45 | 0.231 | 2.19 | 2.74 | ++++ | 0.269 | 0.94 | 1.17 | ++++ |
| 0.50 | 0.320 | 3.59 | 4.49 | +++ | 0.303 | 1.32 | 1.65 | +++ |
| 0.55 | 0.445 | 5.89 | 7.36 | +++ | 0.344 | 1.87 | 2.34 | +++ |
| 0.60 | 0.623 | 9.75 | 12.18 | +++ | 0.394 | 2.66 | 3.33 | +++ |
| 0.65 | 0.886 | 16.5 | 20.6 | + | 0.457 | 3.8 | 4.8 | +++ |
| 0.70 | 1.290 | 28.9 | 36.1 | −− | 0.539 | 5.7 | 7.1 | +++ |
| 0.75 | 1.953 | 53.6 | 67.1 | −−− | 0.653 | 8.8 | 11.0 | +++ |
| 0.80 | 3.138 | 109 | 136 | −−− | 0.822 | 14 | 18 | + |
| 0.85 | 5.574 | 256 | 321 | −−−− | 1.099 | 26 | 33 | ± |
| 0.90 | 11.957 | 800 | 1001 | −−−−− | 1.646 | 59 | 73 | −−− |
| 0.95 | 41.022 | 5032 | 6290 | −−−−− | 3.260 | 213 | 266 | −−− |
| 0.99 | 624.821 | 292040 | 365040 | −−−−− | 15.883 | 3666 | 4583 | −−−−− |
| | Day 27 | | | | Day 30 | | | |
| 0.02 | 0.003 | 0.00118 | 0.00148 | +++++ | 0.011 | 0.00097 | 0.00121 | +++++ |
| 0.05 | 0.009 | 0.0056 | 0.0070 | +++++ | 0.022 | 0.0047 | 0.0058 | +++++ |
| 0.10 | 0.022 | 0.0190 | 0.0238 | +++++ | 0.039 | 0.0162 | 0.0202 | +++++ |
| 0.15 | 0.036 | 0.0407 | 0.0509 | +++++ | 0.057 | 0.0349 | 0.0436 | +++++ |
| 0.20 | 0.054 | 0.072 | 0.090 | +++++ | 0.074 | 0.062 | 0.078 | +++++ |
| 0.25 | 0.076 | 0.116 | 0.144 | +++++ | 0.093 | 0.100 | 0.126 | +++++ |
| 0.30 | 0.102 | 0.174 | 0.218 | ++++ | 0.113 | 0.153 | 0.191 | ++++ |
| 0.35 | 0.134 | 0.254 | 0.317 | ++++ | 0.135 | 0.223 | 0.279 | ++++ |
| 0.40 | 0.173 | 0.36 | 0.45 | ++++ | 0.159 | 0.32 | 0.40 | ++++ |
| 0.45 | 0.222 | 0.50 | 0.63 | ++++ | 0.187 | 0.45 | 0.56 | ++++ |
| 0.50 | 0.283 | 0.70 | 0.88 | ++++ | 0.219 | 0.62 | 0.78 | ++++ |
| 0.55 | 0.361 | 0.97 | 1.22 | +++ | 0.256 | 0.87 | 1.09 | ++++ |
| 0.60 | 0.465 | 1.36 | 1.70 | +++ | 0.300 | 1.23 | 1.53 | +++ |
| 0.65 | 0.605 | 1.9 | 2.4 | +++ | 0.355 | 1.7 | 2.2 | +++ |
| 0.70 | 0.804 | 2.8 | 3.5 | ++ | 0.424 | 2.6 | 3.2 | +++ |
| 0.75 | 1.101 | 4.3 | 5.3 | − | 0.515 | 3.9 | 4.9 | +++ |
| 0.80 | 1.582 | 7 | 9 | −−− | 0.645 | 6 | 8 | +++ |
| 0.85 | 2.459 | 12 | 15 | −−− | 0.846 | 11 | 14 | ++ |
| 0.90 | 4.435 | 26 | 32 | −−−− | 1.214 | 24 | 30 | −− |
| 0.95 | 11.599 | 88 | 110 | −−−−− | 2.174 | 84 | 104 | −−− |
| 0.99 | 99.562 | 1319 | 1648 | −−−−− | 7.879 | 1301 | 1626 | −−−− |
| | Day 37 | | | | Day 41 | | | |
| 0.02 | 0.015 | 0.00014 | 0.00018 | +++++ | 0.025 | 0.00032 | 0.00040 | +++++ |
| 0.05 | 0.027 | 0.0010 | 0.0012 | +++++ | 0.044 | 0.0020 | 0.0025 | +++++ |
| 0.10 | 0.044 | 0.0045 | 0.0057 | +++++ | 0.069 | 0.0083 | 0.0103 | +++++ |
| 0.15 | 0.059 | 0.0117 | 0.0146 | +++++ | 0.092 | 0.0200 | 0.0250 | +++++ |
| 0.20 | 0.073 | 0.024 | 0.030 | +++++ | 0.114 | 0.039 | 0.049 | ++++ |
| 0.25 | 0.089 | 0.043 | 0.054 | +++++ | 0.136 | 0.068 | 0.085 | ++++ |
| 0.30 | 0.104 | 0.072 | 0.090 | ++++ | 0.159 | 0.110 | 0.137 | ++++ |
| 0.35 | 0.121 | 0.114 | 0.143 | ++++ | 0.183 | 0.170 | 0.212 | ++++ |
| 0.40 | 0.139 | 0.18 | 0.22 | ++++ | 0.208 | 0.26 | 0.32 | ++++ |
| 0.45 | 0.160 | 0.27 | 0.34 | ++++ | 0.237 | 0.38 | 0.47 | ++++ |
| 0.50 | 0.182 | 0.40 | 0.51 | ++++ | 0.268 | 0.55 | 0.69 | ++++ |
| 0.55 | 0.209 | 0.61 | 0.76 | ++++ | 0.304 | 0.81 | 1.02 | +++ |
| 0.60 | 0.239 | 0.93 | 1.16 | ++++ | 0.345 | 1.21 | 1.51 | +++ |
| 0.65 | 0.276 | 1.4 | 1.8 | ++++ | 0.394 | 1.8 | 2.3 | +++ |
| 0.70 | 0.323 | 2.3 | 2.9 | +++ | 0.454 | 2.8 | 3.5 | +++ |
| 0.75 | 0.383 | 3.8 | 4.8 | +++ | 0.531 | 4.5 | 5.7 | +++ |
| 0.80 | 0.467 | 7 | 9 | +++ | 0.636 | 8 | 10 | +++ |
| 0.85 | 0.595 | 14 | 17 | +++ | 0.791 | 15 | 19 | ++ |
| 0.90 | 0.825 | 36 | 45 | ++ | 1.059 | 37 | 47 | ± |
| 0.95 | 1.410 | 166 | 207 | −− | 1.699 | 156 | 195 | −−− |
| 0.99 | 4.851 | 4830 | 6037 | −−−− | 4.882 | 3675 | 4594 | −−−− |

| | | Dose (mg/kg) | | |
|---|---|---|---|---|
| Fa | CI | huCBE11 | Gem | Symbol |
| Day 23 | | | | |
| 0.02 | 0.002 | 0.00025 | 0.00032 | ++++ [symbol] |
| 0.05 | 0.005 | 0.0017 | 0.0021 | ++++ [symbol] |
| 0.10 | 0.013 | 0.0073 | 0.0091 | ++++ [symbol] |
| 0.15 | 0.023 | 0.0183 | 0.0229 | ++++ [symbol] |
| 0.20 | 0.035 | 0.037 | 0.046 | ++++ [symbol] |
| 0.25 | 0.049 | 0.065 | 0.081 | ++++ [symbol] |
| 0.30 | 0.067 | 0.106 | 0.133 | ++++ [symbol] |
| 0.35 | 0.088 | 0.167 | 0.209 | ++++ [symbol] |
| 0.40 | 0.114 | 0.26 | 0.32 | +++ [symbol] |
| 0.45 | 0.145 | 0.38 | 0.48 | +++ [symbol] |
| 0.50 | 0.185 | 0.57 | 0.71 | +++ [symbol] |
| 0.55 | 0.235 | 0.85 | 1.06 | +++ [symbol] |
| 0.60 | 0.301 | 1.28 | 1.60 | +++ |
| 0.65 | 0.389 | 2.0 | 2.4 | +++ |
| 0.70 | 0.511 | 3.1 | 3.8 | +++ |
| 0.75 | 0.691 | 5.1 | 6.3 | +++ |
| 0.80 | 0.977 | 9 | 11 | ± |
| 0.85 | 1.484 | 18 | 22 | −−− |
| 0.90 | 2.587 | 45 | 56 | −−− |
| 0.95 | 6.347 | 197 | 246 | −−−− |
| 0.99 | 46.123 | 5200 | 6499 | −−−−− |
| Day 34 | | | | |
| 0.02 | 0.088 | 0.00095 | 0.00119 | +++++ |
| 0.05 | 0.111 | 0.0046 | 0.0057 | ++++ |
| 0.10 | 0.134 | 0.0159 | 0.0198 | ++++ |
| 0.15 | 0.151 | 0.0342 | 0.0428 | ++++ |
| 0.20 | 0.165 | 0.061 | 0.076 | ++++ |
| 0.25 | 0.178 | 0.098 | 0.123 | ++++ |
| 0.30 | 0.190 | 0.149 | 0.187 | ++++ |
| 0.35 | 0.202 | 0.218 | 0.273 | ++++ |
| 0.40 | 0.215 | 0.31 | 0.39 | ++++ |
| 0.45 | 0.227 | 0.44 | 0.55 | ++++ |
| 0.50 | 0.240 | 0.61 | 0.76 | ++++ |
| 0.55 | 0.254 | 0.85 | 1.07 | ++++ |
| 0.60 | 0.269 | 1.20 | 1.50 | ++++ |
| 0.65 | 0.285 | 1.7 | 2.1 | ++++ |
| 0.70 | 0.305 | 2.5 | 3.1 | +++ |
| 0.75 | 0.328 | 3.8 | 4.7 | +++ |
| 0.80 | 0.357 | 6 | 8 | +++ |
| 0.85 | 0.396 | 11 | 14 | +++ |
| 0.90 | 0.456 | 24 | 29 | +++ |
| 0.95 | 0.577 | 82 | 102 | +++ |
| 0.99 | 1.008 | 1267 | 1584 | ± |
| Day 43 | | | | |
| 0.02 | 0.003 | 0.00007 | 0.00009 | +++++ |
| 0.05 | 0.009 | 0.0006 | 0.0008 | +++++ |
| 0.10 | 0.020 | 0.0032 | 0.0040 | +++++ |
| 0.15 | 0.033 | 0.0091 | 0.0113 | +++++ |
| 0.20 | 0.048 | 0.020 | 0.025 | +++++ |
| 0.25 | 0.066 | 0.038 | 0.047 | +++++ |
| 0.30 | 0.086 | 0.066 | 0.083 | +++++ |
| 0.35 | 0.111 | 0.110 | 0.138 | ++++ |
| 0.40 | 0.140 | 0.18 | 0.22 | ++++ |
| 0.45 | 0.176 | 0.28 | 0.35 | ++++ |
| 0.50 | 0.219 | 0.44 | 0.55 | ++++ |
| 0.55 | 0.273 | 0.69 | 0.86 | ++++ |
| 0.60 | 0.343 | 1.09 | 1.37 | +++ |
| 0.65 | 0.433 | 1.8 | 2.2 | +++ |
| 0.70 | 0.557 | 2.9 | 3.7 | +++ |
| 0.75 | 0.735 | 5.2 | 6.4 | ++ |
| 0.80 | 1.009 | 10 | 12 | ± |
| 0.85 | 1.482 | 21 | 27 | −−− |
| 0.90 | 2.469 | 60 | 75 | −−− |
| 0.95 | 5.643 | 321 | 402 | −−−− |
| 0.99 | 35.251 | 12948 | 16185 | −−−−− |

TABLE 34
Determination of Synergism: Simulation of the Combination Indices (CIs):
Mutually Nonexclusive (Totally Independent) Modes of Action

| | | Dose (mg/kg) | | | | Dose (mg/kg) | |
|---|---|---|---|---|---|---|---|
| Fa | CI | huCBE11 | Gem | Symbol | CI | huCBE11 | Gem |
| | Day 16 | | | | | Day 20 | |
| 0.02 | 0.043 | 0.00025 | 0.00031 | +++++ | 0.079 | 0.00161 | 0.00201 |
| 0.05 | 0.022 | 0.0026 | 0.0032 | +++++ | 0.097 | 0.0082 | 0.0103 |
| 0.10 | 0.019 | 0.0161 | 0.0202 | +++++ | 0.119 | 0.0299 | 0.0374 |
| 0.15 | 0.026 | 0.0504 | 0.0630 | +++++ | 0.139 | 0.0664 | 0.0830 |
| 0.20 | 0.038 | 0.119 | 0.148 | +++++ | 0.158 | 0.121 | 0.151 |
| 0.25 | 0.057 | 0.241 | 0.301 | +++++ | 0.179 | 0.199 | 0.249 |
| 0.30 | 0.082 | 0.447 | 0.559 | +++++ | 0.201 | 0.307 | 0.384 |
| 0.35 | 0.118 | 0.784 | 0.980 | ++++ | 0.226 | 0.455 | 0.569 |
| 0.40 | 0.166 | 1.33 | 1.66 | ++++ | 0.255 | 0.66 | 0.82 |
| 0.45 | 0.231 | 2.19 | 2.74 | ++++ | 0.288 | 0.94 | 1.17 |
| 0.50 | 0.321 | 3.59 | 4.49 | +++ | 0.326 | 1.32 | 1.65 |
| 0.55 | 0.446 | 5.89 | 7.36 | +++ | 0.373 | 1.87 | 2.34 |
| 0.60 | 0.624 | 9.75 | 12.18 | +++ | 0.430 | 2.66 | 3.33 |
| 0.65 | 0.887 | 16.5 | 20.6 | + | 0.503 | 3.8 | 4.8 |
| 0.70 | 1.291 | 28.9 | 36.1 | −− | 0.600 | 5.7 | 7.1 |
| 0.75 | 1.954 | 53.6 | 67.1 | −−− | 0.734 | 8.8 | 11.0 |
| 0.80 | 3.140 | 109 | 136 | −−− | 0.934 | 14 | 18 |
| 0.85 | 5.576 | 256 | 321 | −−−− | 1.266 | 26 | 33 |
| 0.90 | 11.961 | 800 | 1001 | −−−−− | 1.930 | 59 | 73 |
| 0.95 | 41.028 | 5032 | 6290 | −−−−− | 3.930 | 213 | 266 |
| 0.99 | 624.846 | 292040 | 365040 | −−−−− | 20.339 | 3666 | 4583 |
| | Day 27 | | | | | Day 30 | |
| 0.02 | 0.003 | 0.00118 | 0.00148 | +++++ | 0.011 | 0.00097 | 0.00121 |
| 0.05 | 0.009 | 0.0056 | 0.0070 | +++++ | 0.022 | 0.0047 | 0.0058 |
| 0.10 | 0.022 | 0.0190 | 0.0238 | +++++ | 0.040 | 0.0162 | 0.0202 |
| 0.15 | 0.037 | 0.0407 | 0.0509 | +++++ | 0.057 | 0.0349 | 0.0436 |
| 0.20 | 0.055 | 0.072 | 0.090 | +++++ | 0.075 | 0.062 | 0.078 |
| 0.25 | 0.077 | 0.116 | 0.144 | +++++ | 0.095 | 0.100 | 0.126 |
| 0.30 | 0.104 | 0.174 | 0.218 | ++++ | 0.115 | 0.153 | 0.191 |
| 0.35 | 0.138 | 0.254 | 0.317 | ++++ | 0.138 | 0.223 | 0.279 |
| 0.40 | 0.179 | 0.36 | 0.45 | ++++ | 0.164 | 0.32 | 0.40 |
| 0.45 | 0.231 | 0.50 | 0.63 | ++++ | 0.194 | 0.45 | 0.56 |
| 0.50 | 0.297 | 0.70 | 0.88 | ++++ | 0.228 | 0.62 | 0.78 |
| 0.55 | 0.384 | 0.97 | 1.22 | +++ | 0.268 | 0.87 | 1.09 |
| 0.60 | 0.501 | 1.36 | 1.70 | +++ | 0.317 | 1.23 | 1.53 |
| 0.65 | 0.663 | 1.9 | 2.4 | +++ | 0.378 | 1.7 | 2.2 |
| 0.70 | 0.899 | 2.8 | 3.5 | + | 0.457 | 2.6 | 3.2 |
| 0.75 | 1.267 | 4.3 | 5.3 | −− | 0.565 | 3.9 | 4.9 |
| 0.80 | 1.894 | 7 | 9 | −−− | 0.723 | 6 | 8 |
| 0.85 | 3.132 | 12 | 15 | −−− | 0.980 | 11 | 14 |
| 0.90 | 6.304 | 26 | 32 | −−−− | 1.488 | 24 | 30 |
| 0.95 | 21.318 | 88 | 110 | −−−−− | 3.050 | 84 | 104 |
| 0.99 | 470.496 | 1319 | 1648 | −−−−− | 19.243 | 1301 | 1626 |
| | Day 37 | | | | | Day 41 | |
| 0.02 | 0.015 | 0.00014 | 0.00018 | +++++ | 0.025 | 0.00032 | 0.00040 |
| 0.05 | 0.027 | 0.0010 | 0.0012 | +++++ | 0.044 | 0.0020 | 0.0025 |
| 0.10 | 0.044 | 0.0045 | 0.0057 | +++++ | 0.070 | 0.0083 | 0.0103 |
| 0.15 | 0.059 | 0.0117 | 0.0146 | +++++ | 0.093 | 0.0200 | 0.0250 |
| 0.20 | 0.074 | 0.024 | 0.030 | +++++ | 0.115 | 0.039 | 0.049 |
| 0.25 | 0.089 | 0.043 | 0.054 | +++++ | 0.138 | 0.068 | 0.085 |
| 0.30 | 0.106 | 0.072 | 0.090 | ++++ | 0.162 | 0.110 | 0.137 |
| 0.35 | 0.123 | 0.114 | 0.143 | ++++ | 0.187 | 0.170 | 0.212 |
| 0.40 | 0.142 | 0.18 | 0.22 | ++++ | 0.214 | 0.26 | 0.32 |
| 0.45 | 0.163 | 0.27 | 0.34 | ++++ | 0.244 | 0.38 | 0.47 |
| 0.50 | 0.187 | 0.40 | 0.51 | ++++ | 0.277 | 0.55 | 0.69 |
| 0.55 | 0.215 | 0.61 | 0.76 | ++++ | 0.316 | 0.81 | 1.02 |
| 0.60 | 0.249 | 0.93 | 1.16 | ++++ | 0.361 | 1.21 | 1.51 |
| 0.65 | 0.289 | 1.4 | 1.8 | ++++ | 0.416 | 1.8 | 2.3 |
| 0.70 | 0.341 | 2.3 | 2.9 | +++ | 0.484 | 2.8 | 3.5 |
| 0.75 | 0.410 | 3.8 | 4.8 | +++ | 0.573 | 4.5 | 5.7 |
| 0.80 | 0.509 | 7 | 9 | +++ | 0.698 | 8 | 10 |
| 0.85 | 0.667 | 14 | 17 | +++ | 0.891 | 15 | 19 |
| 0.90 | 0.972 | 36 | 45 | ± | 1.246 | 37 | 47 |
| 0.95 | 1.874 | 166 | 207 | −−− | 2.216 | 156 | 195 |
| 0.99 | 10.732 | 4830 | 6037 | −−−−− | 9.792 | 3675 | 4594 |

| | | | Dose (mg/kg) | | |
|---|---|---|---|---|---|
| Fa | Symbol | CI | huCBE11 | Gem | Symbol |
| | Day 20 | Day 23 | | | |
| 0.02 | +++++ | 0.002 | 0.00025 | 0.00032 | +++++ |
| 0.05 | +++++ | 0.005 | 0.0017 | 0.0021 | +++++ |
| 0.10 | ++++ | 0.013 | 0.0073 | 0.0091 | +++++ |
| 0.15 | ++++ | 0.023 | 0.0183 | 0.0229 | +++++ |
| 0.20 | ++++ | 0.035 | 0.037 | 0.046 | +++++ |
| 0.25 | ++++ | 0.050 | 0.065 | 0.081 | +++++ |
| 0.30 | ++++ | 0.068 | 0.106 | 0.133 | +++++ |
| 0.35 | ++++ | 0.090 | 0.167 | 0.209 | +++++ |
| 0.40 | ++++ | 0.117 | 0.26 | 0.32 | ++++ |
| 0.45 | ++++ | 0.150 | 0.38 | 0.48 | ++++ |
| 0.50 | +++ | 0.193 | 0.57 | 0.71 | ++++ |
| 0.55 | +++ | 0.248 | 0.85 | 1.06 | ++++ |
| 0.60 | +++ | 0.322 | 1.28 | 1.60 | +++ |
| 0.65 | +++ | 0.424 | 2.0 | 2.4 | +++ |
| 0.70 | +++ | 0.572 | 3.1 | 3.8 | +++ |
| 0.75 | ++ | 0.803 | 5.1 | 6.3 | ++ |
| 0.80 | ± | 1.199 | 9 | 11 | − |
| 0.85 | −− | 1.999 | 18 | 22 | −−− |
| 0.90 | −−− | 4.158 | 45 | 56 | −−−− |
| 0.95 | −−−− | 15.863 | 197 | 246 | −−−−− |
| 0.99 | −−−−− | 555.039 | 5200 | 6499 | −−−−− |
| | Day 30 | Day 34 | | | |
| 0.02 | +++++ | 0.089 | 0.00095 | 0.00119 | +++++ |
| 0.05 | +++++ | 0.112 | 0.0046 | 0.0057 | ++++ |
| 0.10 | +++++ | 0.136 | 0.0159 | 0.0198 | ++++ |
| 0.15 | +++++ | 0.154 | 0.0342 | 0.0428 | ++++ |
| 0.20 | +++++ | 0.169 | 0.061 | 0.076 | ++++ |
| 0.25 | +++++ | 0.183 | 0.098 | 0.123 | ++++ |
| 0.30 | ++++ | 0.196 | 0.149 | 0.187 | ++++ |
| 0.35 | ++++ | 0.209 | 0.218 | 0.273 | ++++ |
| 0.40 | ++++ | 0.222 | 0.31 | 0.39 | ++++ |
| 0.45 | ++++ | 0.235 | 0.44 | 0.55 | ++++ |
| 0.50 | ++++ | 0.249 | 0.61 | 0.76 | ++++ |
| 0.55 | ++++ | 0.265 | 0.85 | 1.07 | ++++ |
| 0.60 | +++ | 0.281 | 1.20 | 1.50 | ++++ |
| 0.65 | +++ | 0.300 | 1.7 | 2.1 | ++++ |
| 0.70 | +++ | 0.322 | 2.5 | 3.1 | +++ |
| 0.75 | +++ | 0.349 | 3.8 | 4.7 | +++ |
| 0.80 | ++ | 0.383 | 6 | 8 | +++ |
| 0.85 | ± | 0.429 | 11 | 14 | +++ |
| 0.90 | −−− | 0.502 | 24 | 29 | +++ |
| 0.95 | −−− | 0.656 | 82 | 102 | +++ |
| 0.99 | −−−−− | 1.262 | 1267 | 1584 | −− |
| | Day 41 | Day 43 | | | |
| 0.02 | +++++ | 0.003 | 0.00007 | 0.00009 | +++++ |
| 0.05 | +++++ | 0.009 | 0.0006 | 0.0008 | +++++ |
| 0.10 | +++++ | 0.020 | 0.0032 | 0.0040 | +++++ |
| 0.15 | +++++ | 0.033 | 0.0091 | 0.0113 | +++++ |
| 0.20 | ++++ | 0.048 | 0.020 | 0.025 | +++++ |
| 0.25 | ++++ | 0.066 | 0.038 | 0.047 | +++++ |
| 0.30 | ++++ | 0.087 | 0.066 | 0.083 | +++++ |
| 0.35 | ++++ | 0.113 | 0.110 | 0.138 | ++++ |
| 0.40 | ++++ | 0.143 | 0.18 | 0.22 | ++++ |
| 0.45 | ++++ | 0.180 | 0.28 | 0.35 | ++++ |
| 0.50 | ++++ | 0.225 | 0.44 | 0.55 | ++++ |
| 0.55 | +++ | 0.283 | 0.69 | 0.86 | ++++ |
| 0.60 | +++ | 0.358 | 1.09 | 1.37 | +++ |
| 0.65 | +++ | 0.458 | 1.8 | 2.2 | +++ |
| 0.70 | +++ | 0.600 | 2.9 | 3.7 | +++ |
| 0.75 | +++ | 0.810 | 5.2 | 6.4 | ++ |
| 0.80 | +++ | 1.155 | 10 | 12 | − |
| 0.85 | ++ | 1.807 | 21 | 27 | −−− |
| 0.90 | −− | 3.411 | 60 | 75 | −−−− |
| 0.95 | −−− | 10.872 | 321 | 402 | −−−−− |
| 0.99 | −−−− | 266.201 | 12948 | 16185 | −−−−− |

In sum, the combination treatment of the LTβ receptor-activating mAb huCBE11 and the chemotherapeutic agent gemcitabine in athymic nude mice implanted subcutaneously with KM-20L2 human colorectal adenocarcinoma showed an effect of combination treatment with huCBE11 and gemcitabine that was determined to be synergistic.

### Example 5

### Antitumor Efficacy of LTβR Agonist in Combination with Plant Alkaloid Chemotherapeutic Agent

Antitumor Efficacy of Combination of huCBE11 with Taxol

In order to determine whether there was a supra-additive effect at treating cancer with the combination treatment of a plant alkaloid chemotherapeutic agent, e.g., Taxol, and LTβ receptor-activating mAb huCBE11, potential syngergistic and potentiating antitumor activity was studied using the antibody/chemotherapy combination in the WiDr xenograft model.

A dosing range study was initially performed to determine the appropriate Taxol and huCBE11 dose(s) for studying combined antitumor effects. The individual agent studies also examined the antitumor efficacy of each agent at inhibiting tumor growth. Athymic nude mice bearing established WiDr tumors were treated with a either saline (control), huCBE11 (5 μg, 50 μg, 100 μg, or 500 μg), or Taxol (doses ranging from 3.13 mg/kg to 25 mg/kg) (saline control n=30; experimental groups n=10 per dose). Tumor size was measured on day 3 and regularly thereafter up to the staging day.

Tumor growth was inhibited in the Taxol alone experimental groups. On Day 50, Taxol had produced a significant inhibition of WiDr human colorectal tumor growth in nude mice at a dose of 25 mg/kg (P<0.0001). The % T/C was below 42% from Days 21 to 50 in the 25 mg/kg group. Tumor growth in the 12.5 mg/kg, 6.25 mg/kg, and 3.13 mg/kg Taxol dose groups did not differ significantly from the vehicle control group and the % T/C was >82% throughout the study in these dose groups. In addition, Taxol produced a significant inhibition of tumor growth at 25 mg/kg (P<0.0001), 18.75 mg/kg (P<0.001), and 6.25 mg/kg (P<0.05) on Day 39 and on Days 13 to 32 in the 12.5 mg/kg group (P<0.05). The % T/C was below 42% on Days 13 to 39 in the 25 mg/kg group and on Days 21 to34 in the 18.75 mg/kg group.

Tumor growth was also inhibited in the huCBE11 experimental groups. On Day 45, huCBE11 produced a significant inhibition of tumor growth at doses of 500 μg (P<0.001), 100 μg (P<0.001), 50 μg (P<0.001), and 5 μg (P<0.05). Similar results were observed on Day 39; tumor weight was significantly less following treatment with 500 μg (P<0.001) and 50 μg (P<0.01) huCBE11 than with the vehicle. The % T/C was below 42% on Days 31 to 45 in the 500 μg group.

In order to determine whether the combination treatment of Taxol and huCBE11 were effective at inhibiting tumor growth, a combination study was performed on athymic nude mice bearing established WiDr tumor cells with established tumors as described above. The combination of huCBE11 and Taxol was determined to be active in the WiDr model based on the NCI activity criteria (% T/C of 42 or less). The combination of huCBE11 500 μg and Taxol 12.5 mg/kg produced significantly greater inhibition of WiDr tumor growth than huCBE11 (500 μg) alone (P<0.05) on Day 39. The % T/C was below 42% in the huCBE11 500 μg plus Taxol 12.5 mg/kg group on Days 24 to 39, in the huCBE11 75 μg plus Taxol 25 mg/kg on Days 13 to 39, in the huCBE11 56.25 μg plus Taxol 18.75 mg/kg on Days 18 to 34, and in the huCBE11 37.5 μg plus Taxol 12.5 mg/kg on Days 27, 34 and 39 (FIG. 3).

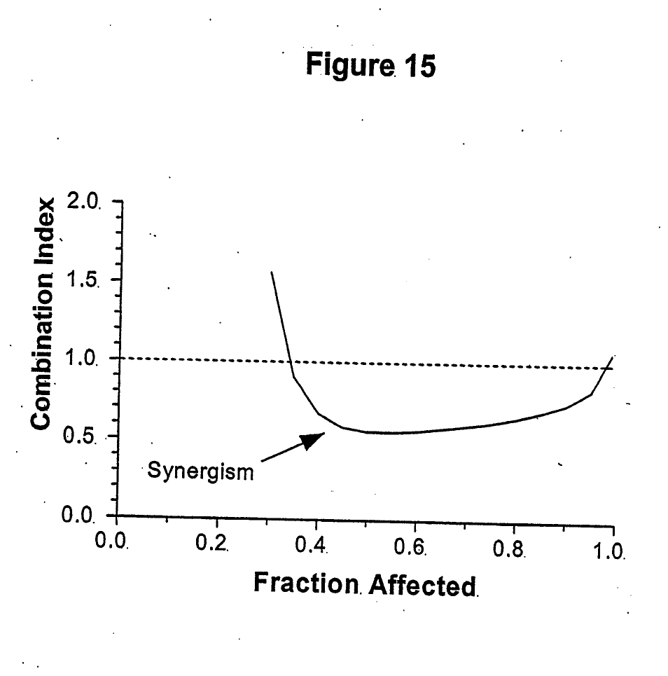

Results from the combination studies (shown in Tables 35-39 and FIGS. 3 and 15) demonstrate that huCBE11 in combination with Taxol significantly decreased tumor volume in treated mice. Antitumor efficacy was determined by comparing each treatment group's tumor volume with the control group's tumor volume. An Fa of 1.000 indicates complete inhibition of the tumor. Table 35 shows the dose-effect relationships for separate and combination treatments of huCBE 11 and Taxol at day 34.

TABLE 35
Dose effect relationship between huCBE11 and Taxol

| | | | | | | Tumor | Volume | |
|---|---|---|---|---|---|---|---|---|
| Treatment | Dose | Units | Cotreatment | Dose | Units | Volume | Decrease | Fa |
| Control | | | | | | 1376.4 | 0.0 | 0.000 |
| Taxol | 6.25 | mg/kg | | | | 1045.2 | 331.2 | 0.241 |
| Taxol | 18.75 | mg/kg | | | | 567.5 | 808.9 | 0.588 |
| Taxol | 25 | mg/kg | | | | 259.1 | 1117.3 | 0.812 |
| huCBE11 | 5 | ug | | | | 1024.0 | 352.4 | 0.256 |
| huCBE11 | 50 | ug | | | | 880.1 | 496.3 | 0.361 |
| huCBE11 | 500 | ug | | | | 735.6 | 640.8 | 0.466 |
| huCBE11 | 18.75 | ug | Taxol | 6.25 | | 639.9 | 736.5 | 0.535 |
| huCBE11 | 37.5 | ug | Taxol | 12.5 | mg/kg | 539.2 | 837.2 | 0.608 |
| huCBE11 | 75 | ug | Taxol | 25 | mg/kg | 157.7 | 1218.7 | 0.885 |
| huCBE11 | 50 | ug | Taxol | 6.25 | mg/kg | 1019.8 | 356.6 | 0.259 |
| huCBE11 | 50 | ug | Taxol | 12.5 | mg/kg | 689.7 | 686.7 | 0.499 |
| huCBE11 | 500 | ug | Taxol | 6.25 | mg/kg | 652.6 | 723.8 | 0.526 |
| huCBE11 | 500 | ug | Taxol | 12.5 | mg/kg | 555.0 | 821.4 | 0.597 |

As treatment of mice bearing the WiDr tumor with Taxol alone produced dose-responsive antitumor efficacy, synergism of the huCBE11 plus Taxol combination could be formally assessed by calculating the Combination Index. Those doses used to assess synergistic drug action in the study were given in a fixed ratio of 0.333:1 (mg/kg Taxol:μg huCBE11). This ratio was based on the ratio of the median effect doses for the 2 agents determined in previous pilot studies. Formal assessment of synergism employed calculation of the Combination Index (CI) using CalcuSyn V1.1 (Biosoft, Cambridge, UK) software for Windows-based dose-effect analysis. For treatments given in combination, a CI equal to 1 indicated additive efficacy. CI less than 1 indicated synergism. CI greater than 1 indicated antagonism. Dose-effect relationships used in CI calculations are shown in Table 36.

TABLE 36
Dose-Effect Relationships of huCBE11 for Synergism Calculations

| Given Separately | | | | Combined (0.333:1) | | |
|---|---|---|---|---|---|---|
| Taxol | Fraction | huCBE11 | Fraction | Taxol | huCBE11 | |
| Dose | Af- | Dose | Af- | Dose | Dose | Fraction |
| (mg/kg) | fected | (μg) | fected | (mg/kg) | (μg) | Affected |
| 6.25 | 0.241 | 5 | 0.256 | 6.25 | 18.8 | 0.535 |
| 18.75 | 0.588 | 50 | 0.361 | 12.5 | 37.5 | 0.608 |
| 25 | 0.812 | 500 | 0.466 | 25 | 75.0 | 0.885 |

Tumor volumes on Day 34 were used to evaluate synergism using the combination index. Potency and shape of the dose-response relation for separate and combination treatments of Taxol and huCBE11 are shown below in Tables 37 and 38, respectively. The combination indices calculated for the exact level of the experimental doses used in this study are given in Table 39.

TABLE 37
Determination of synergism: median effect doses
for synergism of huCBE11 and Taxol

| | | Median Effect Dose (95% Confidence Interval) | |
|---|---|---|---|
| Agent | Dose Units | Given Separately | Combined (0.333:1) |
| Taxol | mg/kg | 12.6 | 6.6 |
| | | (9.4-16.8) | (3.4-12.8) |
| huCBE11 | μg | 932.9 | 19.8 |
| | | (720.7-1207.6) | (10.2-38.4) |

TABLE 38
Shape of Dose-response for separate and combination treatments
Dose-Effect Curve Characteristics

| | | | |
|---|---|---|---|
| Value | Slope | Y-Intercept | R |

Taxol

| | | | |
|---|---|---|---|
| Mean | 1.740 | −1.914 | 0.9717 |
| SEM | 0.423 | 0.501 | |

huCBE11

| | | | |
|---|---|---|---|
| Mean | 0.202 | −0.600 | 0.9993 |
| SEM | 0.008 | 0.014 | |

Taxol + huCBE11

| | | | |
|---|---|---|---|
| Mean | 1.371 | −1.125 | 0.9298 |
| SEM | 0.542 | 0.610 | |

TABLE 39
Combination indices calculated for experimental doses
Combination Index (CI) for Experimental Values

| Taxol | huCBE11 | | Mechanisms of Action | | | |
|---|---|---|---|---|---|---|
| Dose | Dose | Fraction | Mutually Exclusive | | Nonexclusive | |
| (mg/kg) | (ug) | Affected | CI | Synergism | CI | Synergism |

| | | | | | | |
|---|---|---|---|---|---|---|
| 6.25 | 18.75 | 0.535 | 0.468 | +++ | 0.473 | +++ |
| 12.5 | 37.5 | 0.608 | 0.777 | ++ | 0.780 | ++ |
| 25 | 75 | 0.885 | 0.615 | +++ | 0.615 | +++ |

++ moderate synergism
+++ synergism

Because the current study employed drugs that are thought to have entirely independent modes of action, mutually nonexclusive CI values probably apply. Combination doses using 6.25 mg/kg Taxol+18.75 μg huCBE11, 12.5 mg/kg Taxol+37.5 μg huCBE11, and 25 mg/kg Taxol+75 μg huCBE11 showed a synergistic effect. Simulations of the CI over a range of dose levels for the combination are shown in Table 40. Combination doses ranging from 4.9 mg/kg Taxol+14.7 μg huCBE11 (giving 40% inhibition of tumor volume) to 57 mg/kg Taxol+170 μg huCBE11 (giving a 95% inhibition of tumor volume) showed a synergistic effect. Combination Index as a function of fractional effect is shown in FIG. 12.

TABLE 40
Combination Index (CI) Simulations

| | | | | |
|---|---|---|---|---|
| | | Taxol | huCBE11 | |
| Fa | CI | (mg/kg) | (μg) | Symbol |

Mutually Exclusive Mechanisms of Action

| | | | | |
|---|---|---|---|---|
| 0.02 | 287000 | 0.39 | 1.16 | −−−−− |
| 0.05 | 5278 | 0.77 | 2.32 | −−−−− |
| 0.10 | 226 | 1.33 | 3.99 | −−−−− |
| 0.15 | 32 | 1.87 | 5.60 | −−−−− |
| 0.20 | 7.796 | 2.40 | 7.22 | −−−− |
| 0.25 | 2.634 | 2.97 | 8.90 | −−− |
| 0.30 | 1.219 | 3.56 | 10.69 | −− |
| 0.35 | 0.767 | 4.21 | 12.63 | ++ |
| 0.40 | 0.611 | 4.92 | 14.76 | +++ |
| 0.45 | 0.559 | 5.71 | 17.13 | +++ |
| 0.50 | 0.547 | 6.61 | 19.83 | +++ |
| 0.55 | 0.551 | 7.65 | 22.96 | +++ |
| 0.60 | 0.563 | 8.89 | 26.66 | +++ |
| 0.65 | 0.580 | 10.38 | 31.16 | +++ |
| 0.70 | 0.600 | 12.27 | 36.80 | +++ |
| 0.75 | 0.623 | 14.73 | 44.20 | +++ |
| 0.80 | 0.651 | 18.17 | 54.53 | +++ |
| 0.85 | 0.687 | 23.43 | 70.30 | +++ |
| 0.90 | 0.738 | 32.84 | 98.52 | ++ |
| 0.95 | 0.829 | 56.63 | 169.91 | ++ |
| 0.99 | 1.070 | 188.80 | 566.46 | ± |
| 0.02 | 370000 | 0.39 | 1.16 | −−−−− |
| 0.05 | 7036 | 0.77 | 2.32 | −−−−− |
| 0.10 | 310 | 1.33 | 3.99 | −−−−− |
| 0.15 | 45 | 1.87 | 5.60 | −−−−− |
| 0.20 | 10 | 2.40 | 7.22 | −−−− |
| 0.25 | 3.604 | 2.97 | 8.90 | −−−− |
| 0.30 | 1.569 | 3.56 | 10.69 | −−− |
| 0.35 | 0.905 | 4.21 | 12.63 | ± |
| 0.40 | 0.669 | 4.92 | 14.76 | +++ |
| 0.45 | 0.584 | 5.71 | 17.13 | +++ |
| 0.50 | 0.558 | 6.61 | 19.83 | +++ |
| 0.55 | 0.556 | 7.65 | 22.96 | +++ |
| 0.60 | 0.565 | 8.89 | 26.66 | +++ |
| 0.65 | 0.581 | 10.38 | 31.16 | +++ |
| 0.70 | 0.600 | 12.27 | 36.80 | +++ |
| 0.75 | 0.623 | 14.73 | 44.20 | +++ |
| 0.80 | 0.651 | 18.17 | 54.53 | +++ |
| 0.85 | 0.687 | 23.43 | 70.30 | +++ |
| 0.90 | 0.738 | 32.84 | 98.52 | ++ |
| 0.95 | 0.829 | 56.63 | 169.91 | ++ |
| 0.99 | 1.070 | 188.80 | 566.46 | ± |

Those doses used to assess drug potentiation in the current study were not restricted to fixed-ratio combination. Testing for statistically significant potentiation required the calculation of Fa for each animal. Individual tumor volumes (Table 45) were used to calculate fractional inhibition of tumor volume (Fa) for each animal (Table 46). Fa was calculated as (control group mean tumor volume−individual animal tumor volume)÷control group mean tumor volume. The expected additive Fa for a combination treatment was taken to be the sum of mean Fa's from groups receiving either element of the combination. The difference between a combination treatment's actual efficacy and that which would be expected if the treatments were merely additive was also calculated (Table 46). A two-tailed one-sample t-test was used to determine whether the combination treatment produced a mean Fa that was statistically significantly different from the expected additive value (Table 46).

TABLE 45
Individual Tumor Volumes

| | | Taxol | Taxol | | hCBE | hCBE 50 + Taxol | hCBE 50 + Taxol | hCBE 500 + Taxol | hCBE 500 + Taxol |
|---|---|---|---|---|---|---|---|---|---|
| | Control | 6.25 | 12.5 | hCBE 50 | 500 | 6.25 | 12.5 | 6.25 | 12.5 |
| | 1302.1 | 616.0 | 1112.7 | 794.0 | 832.1 | 995.6 | 697.0 | 553.6 | 464.5 |
| | 2573.3 | 985.1 | 1554.9 | 448.6 | 541.3 | 564.3 | 231.8 | 501.0 | 297.4 |
| | 1410.2 | 1675.4 | 1104.0 | 1698.4 | 1059.8 | 953.2 | 111.8 | 647.5 | 624.6 |
| | 1348.2 | 1144.5 | 914.1 | 531.6 | 751.2 | 1868.1 | 463.3 | 401.3 | 483.8 |
| | 1020.3 | 1058.1 | 201.1 | 1322.2 | 1136.7 | 906.8 | 188.0 | 591.6 | 725.8 |
| | 1268.8 | 1425.5 | 1113.1 | 638.5 | 881.2 | 960.2 | 1271.4 | 923.2 | 494.1 |
| | 1321.9 | 796.7 | 1183.1 | 798.8 | 685.7 | 763.9 | 1221.9 | 613.8 | 664.2 |
| | 1318.2 | 860.4 | 1325.2 | 754.3 | 648.1 | 1801.1 | 983.6 | 726.4 | 488.5 |
| | 1629.6 | 730.5 | 1759.5 | 838.2 | 430.1 | 762.2 | 884.1 | 716.5 | 730.8 |
| | 1458.9 | 1159.9 | 1143.0 | 976.8 | 389.8 | 623.0 | 844.0 | 850.8 | 576.6 |
| | 1165.3 | | | | | | | | |
| | 781.6 | | | | | | | | |
| | 1540.0 | | | | | | | | |
| | 1196.5 | | | | | | | | |
| | 1198.1 | | | | | | | | |
| | 1279.7 | | | | | | | | |
| | 975.4 | | | | | | | | |
| | 1079.5 | | | | | | | | |
| | 1666.2 | | | | | | | | |
| | 1994.8 | | | | | | | | |
| Ave.: | 1376.4 | 1045.2 | 1141.1 | 880.1 | 735.6 | 1019.8 | 689.7 | 652.6 | 555.0 |

TABLE 46
Individual Fractional Inhibition of Tumor Volume

| | Taxol | Taxol | | hCBE | hCBE 50 + Taxol | hCBE 50 + Taxol | hCBE 500 + Taxol | hCBE 500 + Taxol |
|---|---|---|---|---|---|---|---|---|
| | 6.25 | 12.5 | hCBE 50 | 500 | 6.25 | 12.5 | 6.25 | 12.5 |
| | 0.552 | 0.192 | 0.423 | 0.395 | 0.277 | 0.494 | 0.598 | 0.663 |
| | 0.284 | −0.130 | 0.674 | 0.607 | 0.590 | 0.832 | 0.636 | 0.784 |
| | −0.217 | 0.198 | −0.234 | 0.230 | 0.307 | 0.919 | 0.530 | 0.546 |
| | 0.168 | 0.336 | 0.614 | 0.454 | −0.357 | 0.663 | 0.708 | 0.649 |
| | 0.231 | 0.854 | 0.039 | 0.174 | 0.341 | 0.863 | 0.570 | 0.473 |
| | −0.036 | 0.191 | 0.536 | 0.360 | 0.302 | 0.076 | 0.329 | 0.641 |
| | 0.421 | 0.140 | 0.420 | 0.502 | 0.445 | 0.112 | 0.554 | 0.517 |
| | 0.375 | 0.037 | 0.452 | 0.529 | −0.309 | 0.285 | 0.472 | 0.645 |
| | 0.469 | −0.278 | 0.391 | 0.688 | 0.446 | 0.358 | 0.479 | 0.469 |
| | 0.157 | 0.170 | 0.290 | 0.717 | 0.547 | 0.387 | 0.382 | 0.581 |
| Ave.: | 0.241 | 0.171 | 0.361 | 0.466 | 0.259 | 0.499 | 0.526 | 0.597 |
| Additive: | | | | | 0.601 | 0.532 | 0.706 | 0.637 |
| Difference: | | | | | −0.342 | −0.033 | −0.180 | −0.040 |
| | Two-Tailed One-Sample T-Test | | | | | | | |
| T-Value: | | | | | −3.286 | −0.340 | −4.981 | −1.291 |
| DF: | | | | | 9 | 9 | 9 | 9 |
| P-Value: | | | | | 0.0094 | 0.7418 | 0.0008 | 0.2289 |

In sum, fixed-ratio combination treatment (0.333:1) using Taxol plus huCBE11 showed synergistic antitumor efficacy on WiDr human colorectal adenocarcinoma.

### EQUIVALENTS

The present invention provides among other things combination therapeutics involving LT-β-R agonists. While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

All publications and patents mentioned herein, including those items listed below, are hereby incorporated by reference in their entirety as if each individual publication or patent was specifically and individually indicated to be incorporated by reference. In case of conflict, the present application, including any definitions herein, will control.

We claim:

1. A method for inhibiting tumor volume comprising administering an effective amount of a lymphotoxin-beta receptor (LT-β-R) agonist and an effective amount of at least one chemotherapeutic agent, wherein the administration of the LT-β-R agonist and the chemotherapeutic agent results in supra-additive inhibition of the tumor.

2. The method of claim 1, wherein the LT-β-R agonist is an anti-LT-β-R antibody.

3. The method of claim 2, wherein the supra-additive inhibition of the tumor is synergistic or potentiated.

4. The method of claim 3, wherein the supra-additive inhibition of the tumor has a combination index of less than 1.00.

5. The method of claim 2, wherein the supra-additive inhibition of the tumor has a P-value of less than 0.05.

6. The method of claim 2, wherein said anti-LT-β-R antibody is a monoclonal antibody.

7. The method of claim 6, wherein said monoclonal antibody is selected from the group consisting of: BKA11, CDH10, BCG6, AGH1, BDA8, CBE11 and BHA10

8. The method of claim 2, wherein said anti-LT-β-R antibody is a humanized antibody or a multivalent anti-LT-β-R antibody.

9. The method of claim 8, wherein said humanized antibody is either huCBE11 or huBHA10.

10. The method of claim 8, wherein said multivalent anti-LT-β-R antibody construct is multispecific.

11. The method of claim 2, wherein the antibody is conjugated to the chemotherapeutic agent.

12. The method of claim 2, wherein the chemotherapeutic agent is selected from the group consisting of an agent that disrupts DNA synthesis, a topoisomerase I inhibitor, an alkylating agent, and a plant alkaloid.

13. The method of claim 12, wherein the agent that disrupts DNA synthesis is a nucleoside analog compound or an anthracycline compound.

14. The method of claim 13, wherein said nucleoside analog compound is gemcitabine.

15. The method of claim 13, wherein the anthracycline compound is adriamycin.

16. The method of claim 12, wherein said topoisomerase I inhibitor is Camptosar.

17. The method of claim 12, wherein said alkylating agent is a platinum compound.

18. The method of claim 17, wherein said platinum compound is either carboplatin or cisplatin.

19. The method of claim 12, wherein said plant alkaloid is a taxane.

20. The method of claim 19, wherein said taxane is Taxol.

21. A method of treating cancer comprising administering an effective amount of a lymphotoxin-beta receptor (LT-β-R) agonist and an effective amount of a chemotherapeutic agent, which upon administration to a subject results in supra-additive inhibition of a tumor.

22. The method of claim 21, wherein the LT-β-R agonist is an anti-LT-β-R antibody.

23. The method of claim 22, wherein the supra-additive inhibition of the tumor is synergistic or potentiated.

24. The method of claim 22, wherein said anti-LT-β-R antibody is a monoclonal antibody.

25. The method of claim 24, wherein said monoclonal antibody is selected from the group consisting of: BKA11, CDH10, BCG6, AGH1, BDA8, CBE11 and BHA10

26. The method of claim 22, wherein said anti-LT-β-R antibody is a humanized antibody or a multivalent anti-LT-β-R antibody.

27. The method of claim 26, wherein said humanized antibody is either huCBE11 or huBHA10.

28. The method of claim 26, wherein said multivalent anti-LT-β-R antibody construct is multispecific.

29. The method of claim 22, wherein the antibody is conjugated to the chemotherapeutic agent.

30. The method of claim 22, wherein the chemotherapeutic agent is selected from the group consisting of an agent that disrupts DNA synthesis, a topoisomerase I inhibitor, an alkylating agent, a plant alkaloid.

31. The method of claim 30, wherein the agent that disrupts DNA synthesis is a nucleoside analog compound or an anthracycline compound.

32. The method of claim 31, wherein said nucleoside analog compound is gemcitabine.

33. The method of claim 31, wherein the anthracycline compound is adriamycin.

34. The method of claim 30, wherein said topoisomerase I inhibitor is Camptosar.

35. The method of claim 30, wherein said alkylating agent is a platinum compound.

36. The method of claim 35, wherein said platinum compound is either carboplatin or cisplatin.

37. The method of claim 30, wherein said plant alkaloid is a taxane.

38. The method of claim 37, wherein said taxane is Taxol.

39. A pharmaceutical composition comprising an effective amount of a LT-β-R agonist, an effective amount of at least one chemotherapeutic agent, and a pharmaceutically acceptable carrier, which upon administration to a subject results in supra-additive inhibition of a tumor.

40. The composition of claim 39, wherein the LT-β-R agonist is an anti-LT-β-R antibody.

41. The composition of claim 40, wherein said anti-LT-β-R antibody is a monoclonal antibody.

42. The composition of claim 41, wherein said monoclonal antibody is selected from the group consisting of: BKA11, CDH10, BCG6, AGH1, BDA8, CBE11 and BHA10

43. The composition of claim 40, wherein said anti-LT-β-R antibody is a humanized antibody or a multivalent anti-LT-β-R antibody.

44. The composition of claim 43, wherein said humanized antibody is either huCBE11 or huBHA10.

45. The composition of claim 43, wherein said multivalent anti-LT-β-R antibody construct is multispecific.

46. The composition of claim 40, wherein the antibody is conjugated to the chemotherapeutic agent.

47. The composition of claim 40, wherein the chemotherapeutic agent is selected from the group consisting of an agent that disrupts DNA synthesis, a topoisomerase I inhibitor, an alkylating agent, a plant alkaloid.


SEQUENCE LISTING (14 sequences)

SEQ ID NO: 1 - DNA, 2094 bp, Artificial Sequence
  Description of Artificial Sequence Heavy chain of hCBE11/hBHA10 bispecific-1 antibody construct
gaggtacaactggtggagtctgggggaggcttagtgaagcctggagggtccctgaggctctcctgtgcagcctctggattcactttcagtgactattacatgtattggtttcgccaggccccgggaaaggggctggagtgggtcgcaaccattagtgatggtggtagttacacctactatccagacagtgtgaaggggcgattcaccatctccagagacaatgccaagaacagcctctacctgcagatgagcagcctgagggctgaggacacagctgtgtattactgcgcaagagaggagaatggtaacttttactactttgactactggggccaagggaccacggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaagactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgcgatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccgggggagggggtggatcaggaggtggcggctcccaggtccaactggtgcagtctggagctgaggtgaagaagcctgggtcctcagtgaaggtgtcctgcaaggcttctggctacactttcacaacctactatttgcactgggtgaggcaggcccctggacagggacttgagtggatgggatggatttatcctggaaatgttcatgctcagtacaatgagaagttcaagggcagggtcacaatcactgcagacaaatccaccagcacagcctacatggagctcagcagcctgaggtctgaagatactgcggtctattactgtgcaagatcctgggaaggttttccttactggggccaagggaccacggtcaccgtctcctcaggtgggggcggatctgggggcggcggatccggtggtggtggtagtgacattcagatgacccagtctcctagctccctgtccgcctcagtaggagacagggtcaccatcacctgcaaggccagtcagaatgtgggtattaatgtagcctggtatcaacagaaaccagggaaggctcctaaatcactgatttcctcggcctcctaccggtacagtggagtcccttccagattcagcggcagtggatctgggacagatttcactctcaccatcagcagcctccagcctgaagacttcgcaacctatttctgtcagcaatatgacacctatccattcacgttcggccagggtaccaaggtggagatcaaatga

SEQ ID NO: 2 - protein, 697 aa, Artificial Sequence
  Description of Artificial Sequence Heavy chain of hCBE11/hBHA10 bispecific-1 antibody construct
EVQLVESGGGLVKPGGSLRLSCAASGFTFSDYYMYWFRQAPGKGLEWVATISDGGSYTYYPDSVKGRFTISRDNAKNSLYLQMSSLRAEDTAVYYCAREENGNFYYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSQVQLVQSGAEVKKPGSSVKVSCKASGYTFTTYYLHWVRQAPGQGLEWMGWIYPGNVHAQYNEKFKGRVTITADKSTSTAYMELSSLRSEDTAVYYCARSWEGFPYWGQGTTVTVSSGGGGSGGGGSGGGGSDIQMTQSPSSLSASVGDRVTITCKASQNVGINVAWYQQKPGKAPKSLISSASYRYSGVPSRFSGSGSGTDFTLTISSLQPEDFATYFCQQYDTYPFTFGQGTKVEIK

SEQ ID NO: 3 - DNA, 645 bp, Artificial Sequence
  Description of Artificial Sequence Light chain of hCBE11/hBHA10 bispecific-1 antibody construct
gatatccagatgacccagtctccatcatccttgtctgcatcggtgggagacagggtcactatcacttgcaaggcgggtcaggacattaaaagctatttaagctggtaccagcagaaaccagggaaagcgcctaagcttctgatctattatgcaacaaggttggcagatggggtcccatcaagattcagtggcagtggatctggtacagattatactctaaccatcagcagcctgcagcctgaggatttcgcaacttattactgtctacagcatggtgagagcccgtggacgttcggtggaggcaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 4 - protein, 194 aa, Artificial Sequence
  Description of Artificial Sequence Light chain of hCBE11/hBHA10 bispecific-1 antibody construct
DIQMTQSPSSLSASVGDRVTITCKAGQDIKSYLSWYQQKPGKAPKLLIYYATRLADGVPSRFSGSGSGTDYTLTISSLQPEDFATYYCLQHGESPWTFGGGTKLEIKRTVAAPSVFIFPPSDEQLKSGTASVVCLLNNFYPREAKVQWKVDNALQSGNSQESVTEQDSKDSTYSLSSTLTLSSPVTKSFNRGEC

SEQ ID NO: 5 - DNA, 2196 bp, Artificial Sequence
  Description of Artificial Sequence hCBE11/hBHA10 bispecific-1 antibody construct
gaggtacaactggtggagtctgggggaggcttagtgaagcctggagggtccctgaggctctcctgtgcagcctctggattcactttcagtgactattacatgtattggtttcgccaggccccgggaaaggggctggagtgggtcgcaaccattagtgatggtggtagttacacctactatccagacagtgtgaaggggcgattcaccatctccagagacaatgccaagaacagcctctacctgcagatgagcagcctgagggctgaggacacagctgtgtattactgcgcaagagaggagaatggtaacttttactactttgactactggggccaagggaccacggtcaccgtctcctctgggggcggggggtccgggggaggcgggtcgggaggtggcggaagtgatatccagatgacccagtctccatcatccttgtctgcatcggtgggagacagggtcactatcacttgcaaggcgggtcaggacattaaaagctatttaagctggtaccagcagaaaccagggaaagcgcctaagcttctgatctattatgcaacaaggttggcagatggggtcccatcaagattcagtggcagtggatctggtacagattatactctaaccatcagcagcctgcagcctgaggatttcgcaacttattactgtctacagcatggtgagagcccgtggacgttcggtggaggcaccaagctggagatcaaagggggtggtggttcaggaggtggaggatccgagcccaaatctagtgacaagactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgcgatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccgggggagggggtggatcaggaggtggcggctcccaggtccaactggtgcagtctggagctgaggtgaagaagcctgggtcctcagtgaaggtgtcctgcaaggcttctggctacactttcacaacctactatttgcactgggtgaggcaggcccctggacagggacttgagtggatgggatggatttatcctggaaatgttcatgctcagtacaatgagaagttcaagggcagggtcacaatcactgcagacaaatccaccagcacagcctacatggagctcagcagcctgaggtctgaagatactgcggtctattactgtgcaagatcctgggaaggttttccttactggggccaagggaccacggtcaccgtctcctcaggtgggggcggatctgggggcggcggatccggtggtggtggtagtgacattcagatgacccagtctcctagctccctgtccgcctcagtaggagacagggtcaccatcacctgcaaggccagtcagaatgtgggtattaatgtagcctggtatcaacagaaaccagggaaggctcctaaatcactgatttcctcggcctcctaccggtacagtggagtcccttccagattcagcggcagtggatctgggacagatttcactctcaccatcagcagcctccagcctgaagacttcgcaacctatttctgtcagcaatatgacacctatccattcacgttcggccagggtaccaaggtggagatcaaatga

SEQ ID NO: 6 - protein, 731 aa, Artificial Sequence
  Description of Artificial Sequence hCBE11/hBHA10 bispecific-1 antibody construct
EVQLVESGGGLVKPGGSLRLSCAASGFTFSDYYMYWFRQAPGKGLEWVATISDGGSYTYYPDSVKGRFTISRDNAKNSLYLQMSSLRAEDTAVYYCAREENGNFYYFDYWGQGTTVTVSSGGGGSGGGGSGGGGSDIQMTQSPSSLSASVGDRVTITCKAGQDIKSYLSWYQQKPGKAPKLLIYYATRLADGVPSRFSGSGSGTDYTLTISSLQPEDFATYYCLQHGESPWTFGGGTKLEIKGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSQVQLVQSGAEVKKPGSSVKVSCKASGYTFTTYYLHWVRQAPGQGLEWMGWIYPGNVHAQYNEKFKGRVTITADKSTSTAYMELSSLRSEDTAVYYCARSWEGFPYWGQGTTVTVSSGGGGSGGGGSGGGGSDIQMTQSPSSLSASVGDRVTITCKASQNVGINVAWYQQKPGKAPKSLISSASYRYSGVPSRFSGSGSGTDFTLTISSLQPEDFATYFCQQYDTYPFTFGQGTKVEIK

SEQ ID NO: 7 - DNA, 2106 bp, Artificial Sequence
  Description of Artificial Sequence Heavy chain of hCBE11 monospecific-1 antibody construct
gaggtacaactggtggagtctgggggaggcttagtgaagcctggagggtccctgaggctctcctgtgcagcctctggattcactttcagtgactattacatgtattggtttcgccaggccccgggaaaggggctggagtgggtcgcaaccattagtgatggtggtagttacacctactatccagacagtgtgaaggggcgattcaccatctccagagacaatgccaagaacagcctctacctgcagatgagcagcctgagggctgaggacacagctgtgtattactgcgcaagagaggagaatggtaacttttactactttgactactggggccaagggaccacggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaagactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgcgatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggggggggaggtggatcaggaggtggcggctccgaggtacaactggtggagtctgggggaggcttagtgaagcctggagggtccctgaggctctcctgtgcagcctctggattcactttcagtgactattacatgtattggtttcgccaggcaccgggaaaggggctggagtgggtcgcaaccattagtgatggtggtagttacacctactatccagacagtgtgaaggggcgattcaccatctccagagacaatgccaagaacagcctctacctgcagatgagcagcctgagggctgaggacacagctgtgtattactgcgcaagagaggagaatggtaacttttactactttgactactggggccaagggaccacggtcaccgtctcctctgggggcggggggtccgggggaggcgggtcgggaggtggcggaagtgatatccagatgacccagtctccatcatccttgtctgcatcggtgggagacagggtcactatcacttgcaaggcgggtcaggacattaaaagctatttaagctggtaccagcagaaaccagggaaagcgcctaagcttctgatctattatgcaacaaggttggcagatggggtcccatcaagattcagtggcagtggatctggtacagattatactctaaccatcagcagcctgcagcctgaggatttcgcaacttattactgtctacagcatggtgagagcccgtggacgttcggtggaggcaccaagctggagatcaaatga

SEQ ID NO: 8 - protein, 701 aa, Artificial Sequence
  Description of Artificial Sequence Heavy chain of hCBE11 monospecific-1 antibody construct
EVQLVESGGGLVKPGGSLRLSCAASGFTFSDYYMYWFRQAPGKGLEWVATISDGGSYTYYPDSVKGRFTISRDNAKNSLYLQMSSLRAEDTAVYYCAREENGNFYYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEVQLVESGGGLVKPGGSLRLSCAASGFTFSDYYMYWFRQAPGKGLEWVATISDGGSYTYYPDSVKGRFTISRDNAKNSLYLQMSSLRAEDTAVYYCAREENGNFYYFDYWGQGTTVTVSSGGGGSGGGGSGGGGSDIQMTQSPSSLSASVGDRVTITCKAGQDIKSYLSWYQQKPGKAPKLLIYYATRLADGVPSRFSGSGSGTDYTLTISSLQPEDFATYYCLQHGESPWTFGGGTKLEIK

SEQ ID NO: 9 - DNA, 2208 bp, Artificial Sequence
  Description of Artificial Sequence hCBE11 monospecific-2 antibody construct
gaggtacaactggtggagtctgggggaggcttagtgaagcctggagggtccctgaggctctcctgtgcagcctctggattcactttcagtgactattacatgtattggtttcgccaggccccgggaaaggggctggagtgggtcgcaaccattagtgatggtggtagttacacctactatccagacagtgtgaaggggcgattcaccatctccagagacaatgccaagaacagcctctacctgcagatgagcagcctgagggctgaggacacagctgtgtattactgcgcaagagaggagaatggtaacttttactactttgactactggggccaagggaccacggtcaccgtctcctctgggggcggggggtccgggggaggcgggtcgggaggtggcggaagtgatatccagatgacccagtctccatcatccttgtctgcatcggtgggagacagggtcactatcacttgcaaggcgggtcaggacattaaaagctatttaagctggtaccagcagaaaccagggaaagcgcctaagcttctgatctattatgcaacaaggttggcagatggggtcccatcaagattcagtggcagtggatctggtacagattatactctaaccatcagcagcctgcagcctgaggatttcgcaacttattactgtctacagcatggtgagagcccgtggacgttcggtggaggcaccaagctggagatcaaagggggtggtggttcaggaggtggaggatccgagcccaaatctagtgacaagactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgcgatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggggggggaggtggatcaggaggtggcggctccgaggtacaactggtggagtctgggggaggcttagtgaagcctggagggtccctgaggctctcctgtgcagcctctggattcactttcagtgactattacatgtattggtttcgccaggcaccgggaaaggggctggagtgggtcgcaaccattagtgatggtggtagttacacctactatccagacagtgtgaaggggcgattcaccatctccagagacaatgccaagaacagcctctacctgcagatgagcagcctgagggctgaggacacagctgtgtattactgcgcaagagaggagaatggtaacttttactactttgactactggggccaagggaccacggtcaccgtctcctctgggggcggggggtccgggggaggcgggtcgggaggtggcggaagtgatatccagatgacccagtctccatcatccttgtctgcatcggtgggagacagggtcactatcacttgcaaggcgggtcaggacattaaaagctatttaagctggtaccagcagaaaccagggaaagcgcctaagcttctgatctattatgcaacaaggttggcagatggggtcccatcaagattcagtggcagtggatctggtacagattatactctaaccatcagcagcctgcagcctgaggatttcgcaacttattactgtctacagcatggtgagagcccgtggacgttcggtggaggcaccaagctggagatcaaatga

SEQ ID NO: 10 - protein, 735 aa, Artificial Sequence [sequence omitted]

SEQ ID NO: 11 - DNA, 1407 bp, Artificial Sequence
  Description of Artificial Sequence CBE11 pentameric heavy chain antibody construct
gaggtacaactggtggagtctgggggaggcttagtgaagcctggagggtccctgaaactctcctgtgcagcctctggattcactttcagtgactattacatgtattggtttcgccagactccggaaaagaggctggagtgggtcgcaaccattagtgatggtggtagttacacctactatccagacagtgtgaaggggcgattcaccatctccagagacaatgccaagaacaacctgtacctgcaaatgagcagtctgaagtctgaggacacagccatgtattactgtgtaagagaggagaatggtaacttttactactttgactactggggccaagggaccacggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaagactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctaccgggaaacccaccctgtacaacgtgtccctggtcatgtccgacacagctggcacctgctactga

SEQ ID NO: 12 - protein, 468 aa, Artificial Sequence
  Description of Artificial Sequence CBE11 pentameric heavy chain antibody construct
EVQLVESGGGLVKPGGSLKLSCAASGFTFSDYYMYWFRQTPEKRLEWVATISDGGSYTYYPDSVKGRFTISRDNAKNNLYLQMSSLKSEDTAMYYCVREENGNFYYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSTGKPTLYNVSLVMSDTAGTCY

SEQ ID NO: 13 - DNA, 645 bp, Artificial Sequence
  Description of Artificial Sequence CBE 11 chimeric light chain antibody construct
gatattaagatgacccagtctccatcctccatgtatgcatcgctgggagagagagtcactatcacttgcaaggcgggtcaggacattaaaagctatttaagctggtaccagcagaaaccatggaaatctcctaagatcctgatctattatgcaacaaggttggcagatggggtcccatcaagattcagtggcagtggatctgggcaagattattctctaaccatcagcagcctggagtctgacgatacagcaacttattactgtctacagcatggtgagagcccgtggacgttcggtggaggcaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 14 - protein, 214 aa, Artificial Sequence
  Description of Artificial Sequence CBE 11 chimeric light chain antibody construct
DIKMTQSPSSMYASLGERVTITCKAGQDIKSYLSWYQQKPWKSPKILIYYATRLADGVPSRFSGSGSGQDYSLTISSLESDDTATYYCLQHGESPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC